(19) United States
(12) Patent Application Publication
(10) Pub. No.: US 2024/0065282 A1
(43) Pub. Date: Feb. 29, 2024

(54) MICELLE AND MICELLE-LIKE COMPOSITIONS AND RELATED METHODS

(71) Applicant: New Culture Inc., San Leandro, CA (US)

(72) Inventors: Inja RADMAN, Oakland, CA (US); Rebecca REITH, Berkeley, CA (US); Neil ADAMES, San Leandro, CA (US); Patrick STODDARD, San Francisco, CA (US); Dilrajkaur PANFAIR, San Jose, CA (US)

(73) Assignee: New Culture Inc., San Leandro, CA (US)

(21) Appl. No.: 18/035,263

(22) PCT Filed: Nov. 4, 2021
(86) PCT No.: PCT/US2021/058029

§ 371 Date: May 3, 2023

Related U.S. Application Data
(60) Provisional application No. 63/109,837, filed on Nov. 4, 2020.
(60) Provisional application No. 63/109,851, filed on Nov. 4, 2020.

(51) Int. Cl. A23C 20/00; C12N 9/10; C12N 15/70; C12N 15/74; A23J 3/10; A23J 3/34; A23J 3/22; A23J 3/28

(52) U.S. Cl. CPC A23C 20/00; C12Y 203/02013; C12N 9/1044; C12N 15/70; C12N 15/74; A23J 3/10; A23J 3/344; A23J 3/222; A23J 3/225; A23J 3/285; C12N 2800/101

(57) ABSTRACT

Provided herein are micelle and micelle-like compositions and the methods of making the same. The micelle and micelle-like compositions may be used to form dairy-like products.

### CROSS-REFERENCE

This application claims the benefit of U.S. Provisional Application Nos. 63/109,837, and 63/109,851, both filed Nov. 4, 2020, which applications are incorporated herein by reference in their entirety.

### SEQUENCE LISTING

The instant application contains a Sequence Listing which has been submitted electronically in ASCII format and is hereby incorporated by reference in its entirety. Said ASCII copy, created on Nov. 2, 2021, is named 56127-704_601_SL.txt and is 122,328 bytes in size.

### BACKGROUND

The clean food space is comprised of both plant-based and cell-based foods. Cell-based food is a large umbrella term that includes culturing muscle and fat cells to replace slaughtered meat and culturing bioengineered organisms to express recombinant animal proteins to replace other animal products such as dairy and eggs. The need to find an alternate source of animal protein comes from the inefficiencies and unsustainability of current animal food production.

### SUMMARY

In one aspect, a micelle composition is provided comprising an alpha casein and a kappa casein, wherein at least one of the alpha casein and the kappa casein is a recombinant protein, wherein the alpha casein, the kappa casein, or both the alpha casein and the kappa casein comprise a non-native post-translational modification feature, and wherein the alpha casein and the kappa casein are associated in micelles. In some cases, at least a portion of micelles of the micelle composition comprises cross-linked casein protein. In some cases, the micelle composition comprises micelles having intra-micellar crosslinking. In some cases, at least a portion of the alpha casein and the kappa casein are in micellar form. In some cases, at least a portion of micelles of the micelle composition comprises intra-micellar crosslinking, and wherein a majority of the micelles are not comprised within an inter-micellar crosslinked structure. In some cases, the non-native post-translational modification feature comprises a reduction in phosphorylation, a lack of phosphorylation, or a modification of one or more sites of phosphorylation of the alpha casein. In some cases, the non-native post-translational modification feature comprises a reduction in glycosylation, a lack of glycosylation, or a modification of one or more sites of glycosylation of the kappa casein. In some cases, the alpha casein is a recombinant protein. In some cases, the kappa casein is a recombinant protein. In some cases, both the alpha casein and the kappa casein are recombinant proteins. In some cases, the alpha casein comprises a mixture of native alpha casein and one or more altered forms thereof. In some cases, the one or more altered forms thereof is a truncated alpha casein (e.g., truncated relative to a native alpha casein). In some cases, the kappa casein comprises a mixture of a native kappa casein and one or more altered forms thereof. In some cases, the one or more altered forms thereof is a truncated kappa casein (e.g., truncated relative to a native kappa casein). In some cases, the alpha casein, the kappa casein, or both the alpha casein and the kappa casein are produced in a recombinant host cell selected from the group consisting of: a microbial cell, a plant cell, and a mammalian cell; optionally, wherein the recombinant host cell is a microbial cell. In some cases, the microbial cell is a bacterium. In some cases, the micelle composition further comprises a beta casein or a derivative thereof. In some cases, the micelle composition further comprises a gamma casein. In some cases, the micelles do not comprise a beta casein or derivative thereof. In some cases, a ratio of the alpha casein to the kappa casein in the micelle composition is from about 1:1 to about 15:1. In some cases, a ratio of the alpha casein to the kappa casein in the micelle composition is about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In some cases, the alpha casein comprises only alpha-S1 casein. In some cases, the alpha casein comprises only alpha-S2 casein. In some cases, the alpha casein comprises an amino acid sequence of cow, human, sheep, goat, buffalo, bison, horse, or camel alpha casein. In some cases, the alpha casein protein has an amino acid sequence comprising any one of SEQ ID NOs: 1-39 or 64-72 or a variant thereof having an amino acid sequence with at least 80% sequence identity to any one of SEQ ID NOs: 1-39 or 64-72. In some cases, the kappa casein comprises an amino acid sequence of cow, human, sheep, goat, buffalo, bison, horse, or camel kappa casein. In some cases, the kappa casein has an amino acid sequence comprising any one of SEQ ID NOs: 40-60, or a variant thereof having an amino acid sequence with at least 80% sequence identity to any one of SEQ ID NOs: 40-60. In some cases, the alpha casein and the kappa casein are from different mammalian species. In some cases, the alpha casein comprises an amino acid sequence of bovine alpha casein, and the kappa casein comprises an amino acid sequence of sheep kappa casein. In some cases, the micelle composition comprises a population of micelles with an average or mean size from about 200 nm to about 400 nm. In some cases, the micelle composition comprises a population of micelles with an average or mean size of about 300 nm. In some cases, the micelle composition comprises a population of micelles with an average or mean size of about 200 nm. In some cases, the micelle composition further comprises at least one salt selected from the group consisting of a calcium salt, a citrate salt, and a phosphate salt. In some cases, the micelle composition is susceptible to renneting. In some cases, the micelle composition, after renneting, forms stable and strong curds (e.g., as measured by a tube inversion test).

In another aspect, a micelle-like composition is provided comprising a kappa casein in the absence of alpha casein and beta casein, wherein the kappa casein forms a micelle-like structure. In some cases, the kappa casein comprises intra-micellar crosslinking between kappa casein molecules. In some cases, the kappa casein has an amino acid sequence comprising any one of SEQ ID NOs: 40-60, or a variant thereof comprising an amino acid sequence with at least 80% sequence identity to any one of SEQ ID NOs: 40-60. In some cases, kappa casein comprises an amino acid sequence of cow, human, sheep, goat, buffalo, bison, horse, or camel kappa casein protein. In some cases, the kappa casein has an amino acid sequence comprising any one of SEQ ID NOs: 43-45, or a variant thereof comprising an amino acid sequence with at least 80% sequence identity to any one of SEQ ID NOs: 43-45. In some cases, the kappa casein comprises an amino acid sequence of sheep kappa casein protein. In some cases, the kappa casein comprises a mixture of a native kappa casein and one or more altered forms thereof. In some cases, the one or more altered forms thereof is a truncated kappa casein (e.g., truncated relative to a native kappa casein). In some cases, the kappa casein comprises a first kappa casein protein and a second kappa casein protein. In some cases, the first kappa casein protein and the second kappa casein protein are from different mammalian species. In some cases, the micelle-like composition comprises a population of micelle-like structures with an average or mean size from about 150 nm to about 700 nm. In some cases, the micelle-like composition comprises a population of micelle-like structures with an average or mean size of about 400 nm. In some cases, the micelle-like composition comprises a population of micelle-like structures with an average or mean size of about 100 nm to about 250 nm. In some cases, the micelle-like composition comprises a population of micelle-like structures with an average or mean size of about 600 nm to about 700 nm. In some cases, the micelle-like composition further comprises at least one salt selected from the group consisting of a calcium salt, a citrate salt, and a phosphate salt. In some cases, the micelle-like composition is susceptible to renneting. In some cases, the micelle-like composition, after renneting, forms stable and strong curds (e.g., as measured by a tube inversion test).

In another aspect, a dairy-like product is provided comprising the micelle composition according to any one of the preceding or the micelle-like composition of any one of the preceding. In some cases, the micelle composition or micelle-like composition does not include any additional dairy protein. In some cases, the dairy-like product is incorporated into an edible composition. In some cases, the edible composition does not include any animal-obtained dairy protein. In some cases, the dairy-like product is selected from the group consisting of milk, yogurt, curd, cheese, cream, and ice cream. In some cases, the dairy-like product is curd. In some cases, the dairy-like product comprises a cheese selected from the group consisting of a soft cheese, a hard cheese, a pasta filata cheese, and an aged cheese. In some cases, the cheese has a fat content from about 0% to about 50% and the fat is not an animal-obtained fat. In some cases, the cheese has a sugar content from about 0% to about 10% and the sugar is derived from a plant-based source. In some cases, the cheese is selected from the group consisting of pasta filata-like cheese, paneer, cream cheese, and cottage cheese. In some cases, the cheese is an aged or matured cheese selected from the group consisting of cheddar, Swiss, gouda, brie, camembert, feta, halloumi, edam, manchego, colby, muenster, blue cheese, or parmesan. In some cases, the cheese is mozzarella. In some cases, the moisture retention of the cheese is from about 30% to about 80%. In some cases, the cheese is capable of one or more of stretching when heated, melting when heated, or browning when heated. In some cases, the texture of the cheese is comparable to an animal-obtained dairy cheese. In some cases, the hardness of the cheese is comparable to an animal-obtained dairy cheese. In some cases, the hardness of the cheese is reduced as compared to an animal-obtained dairy cheese. In some cases, the melt of the cheese is comparable to an animal-obtained dairy cheese. In some cases, the melt of the cheese is improved compared to an animal-obtained dairy cheese. In some cases, the stretch of the cheese is comparable to an animal-obtained dairy cheese. In some cases, the stretch of the cheese is improved compared to an animal-obtained dairy cheese. In some cases, the dairy-like product comprises the micelle-like composition of any one of the preceding, and the yield of the cheese is improved as compared to a comparable dairy-like product without the intra-micellar crosslinking between kappa casein molecules. In some cases, the dairy-like product comprises the micelle-like composition of any one of the preceding, and the melt of the cheese is improved as compared to a comparable dairy-like product without the intra-micellar crosslinking between kappa casein molecules. In some cases, the dairy-like product comprises the micelle-like composition of any one of the preceding, and the stretch of the cheese is improved as compared to a comparable dairy-like product without the intra-micellar crosslinking between kappa casein molecules.

In another aspect, a powder is provided comprising the micelle composition of any one of the preceding or the micelle-like composition of any one of the preceding. In some cases, the casein content of the powder is from about 50% to about 90%.

In yet another aspect, a method of making a dairy-like ingredient is provided comprising: (a) providing an alpha casein and a kappa casein, wherein the alpha casein, the kappa casein, or both the alpha casein and the kappa casein comprise a non-native post-translational modification feature; (b) inducing micelle formation; and (c) providing a crosslinking agent under conditions for inducing intra-micellar crosslinking, wherein the method produces micelles comprising the alpha casein and the kappa casein in a form suitable for a dairy-like ingredient.

In yet another aspect, a method of making a dairy-like ingredient is provided comprising: (a) providing an alpha casein, wherein the alpha casein comprises a non-native posttranslational modification feature; (b) providing a crosslinking agent under conditions for crosslinking the alpha casein; and (c) mixing kappa casein with the crosslinked alpha casein under conditions to induce micelle formation, wherein the method produces micelles comprising the alpha casein and the kappa casein in a form suitable for a dairy-like ingredient.

In any of the preceding methods, the alpha casein and kappa casein are incubated together prior to adding the crosslinking agent. In any of the preceding methods, the crosslinking agent is added from about 30 minutes to about 24 hours after the alpha casein and kappa casein are incubated together. In any of the preceding methods, the crosslinking agent is added from about 1 hour to about 12 hours after the alpha casein and kappa casein are incubated together. In any of the preceding methods, the crosslinking agent is added prior to the step of inducing micelle formation. In any of the preceding methods, the crosslinking agent is added subsequent to the step of inducing micelle formation. In any of the preceding methods, the crosslinking agent is a transglutaminase. In any of the preceding methods, the non-native posttranslational modification feature comprises a reduction in phosphorylation, a lack of phosphorylation, or a modification of one or more sites of phosphorylation on the alpha casein. In any of the preceding methods, the non-native post-translational modification feature comprises a reduction in glycosylation, a lack of glycosylation, or a modification of one or more sites of glycosylation on the kappa casein. In any of the preceding methods, the method further comprises producing the alpha casein, the kappa casein, or both in a recombinant host cell selected from the group consisting of a microbial cell, a plant cell, and a mammalian cell; optionally, wherein the recombinant host cell is a microbial cell. In any of the preceding methods, the recombinant host cell is a microbial cell. In any of the preceding methods, the microbial cell is selected from the group consisting of Lactococci sp., Lactococcus lactis, Bacillus subtilis, Bacillus amyloliquefaciens, Bacillus licheniformis, Bacillus megaterium, Mycobacterium smegmatis, Rhodococcus erythropolis, Corynebacterium glutamicum, Lactobacilli sp., Lactobacillus fermentum, Lactobacillus casei, Lactobacillus acidophilus, Lactobacillus plantarum, Synechocystis sp. 6803, and Escherichia coli. In any of the preceding methods, the dairy-like ingredient is susceptible to renneting. In any of the preceding methods, the conditions to induce micelle formation include the addition of a salt. In any of the preceding methods, the micelles are comprised in a liquid colloid. In any of the preceding methods, the method further comprises the step of forming a dairy-like product from the liquid colloid. In any of the preceding methods, the dairy-like product comprises milk, cream, curd, cheese, yogurt, or ice cream. In any of the preceding methods, the method further comprises subjecting the liquid colloid to a first condition to form coagulates. In any of the preceding methods, the first condition is the addition of acid or acidification of the liquid colloid with a microorganism. In any of the preceding methods, the method further comprises subjecting the coagulates to a hot water treatment and optionally stretching to form a filata type cheese. In any of the preceding methods, the method further comprises subjecting the coagulates to a renneting agent to form a rennetted curd. In any of the preceding methods, the renneting agent is a microbially-derived chymosin enzyme. In any of the preceding methods, the method further comprises cooking, aging, and maturing the rennetted curd to form an aged or matured cheese-like composition. In any of the preceding methods, the method further comprises subjecting the rennetted curd to a hot water treatment and optionally stretching to form a filata-type cheese. In any of the preceding methods, the method further comprises forming a yogurt from the liquid colloid. In any of the preceding methods, forming the yogurt comprises optionally heating and then cooling the liquid colloid, and acidifying the liquid colloid with a microorganism. In any of the preceding methods, the microorganism comprises one or more of Lactobacillus delbrueckii subsp. bulgaricus, Streptococcus thermophilus, a Lactobacilli, or a Bifzdobacteria species. In any of the preceding methods, the micelles do not include beta casein protein. In any of the preceding methods, the dairy-like ingredient does not include any additional dairy protein. In any of the preceding methods, the dairy-like ingredient does not include any animal-obtained dairy protein. In any of the preceding methods, the dairy-like product comprises a fat, a sugar, a flavoring, or a colorant. In any of the preceding methods, the dairy-like ingredient is in a powder form. In any of the preceding methods, the method further comprises drying, lyophilizing, drum drying, or spray-drying to produce the powder form. In any of the preceding methods, the ratio of alpha casein to kappa casein is from about 1:1 to about 15:1. In any of the preceding methods, the ratio of alpha casein to kappa protein is about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1. In any of the preceding methods, the alpha casein comprises only alpha-S1 casein. In any of the preceding methods, the alpha casein comprises only alpha-S2 casein. In any of the preceding methods, the alpha casein has an amino acid sequence comprising any one of SEQ ID NOs: 1-39 or 64-72, or a variant thereof comprising an amino acid sequence with at least 80% sequence identity to any one of SEQ ID NOs: 1-39 or 64-72. In any of the preceding methods, the alpha casein comprises an amino acid sequence of cow, human, sheep, goat, buffalo, bison, horse, or camel alpha casein. In any of the preceding methods, the kappa casein has an amino acid sequence comprising any one of SEQ ID NOs: 40-60, or a variant thereof comprising an amino acid sequence with at least 80% sequence identity to any one of SEQ ID NOs: 40-60. In any of the preceding methods, the kappa casein comprises an amino acid sequence of cow, human, sheep, goat, buffalo, bison, horse, or camel kappa casein protein. In any of the preceding methods, the kappa casein has an amino acid sequence comprising any one of SEQ ID NOs: 43-45, or a variant thereof comprising an amino acid sequence with at least 80% sequence identity to any one of SEQ ID NOs: 43-45. In any of the preceding methods, the kappa casein comprises an amino acid sequence of sheep kappa casein protein. In any of the preceding methods, the alpha casein comprises a mixture of native alpha casein and one or more altered forms thereof. In any of the preceding methods, the one or more altered forms thereof is a truncated alpha casein (e.g., truncated relative to a native alpha casein). In any of the preceding methods, the kappa casein comprises a mixture of a native kappa casein and one or more altered forms thereof. In any of the preceding methods, the one or more altered forms thereof is a truncated kappa casein (e.g., truncated relative to a native kappa casein). In any of the preceding methods, the alpha casein and the kappa casein are from different mammalian species. In any of the preceding methods, the alpha casein comprises an amino acid sequence of bovine alpha casein, and the kappa casein comprises an amino acid sequence of sheep kappa casein.

In yet another aspect, a coagulated composition formed by the method of any one of the preceding methods is provided.

In yet another aspect, a renneted curd composition formed by the method of any one of the preceding methods is provided.

In yet another aspect, a dairy-like composition formed by the method of any one of the preceding methods is provided. In some cases, the dairy-like composition is selected from the group consisting of milk, cream, curd, cheese, yogurt, and ice cream. In some cases, the dairy-like composition is selected from the group consisting of pasta filata-like cheese, paneer, cream cheese, cottage cheese, cheddar, Swiss, gouda, and mozzarella.

In another aspect, a method of making a dairy-like ingredient is provided comprising providing a kappa casein in the absence of any alpha casein or beta casein under conditions such that the kappa casein forms a micelle-like structure in a form suitable for a dairy-like ingredient. In some cases, the method further comprises providing a crosslinking agent under conditions for crosslinking the kappa casein. In some cases, the micelle-like structure comprises intra-micellar crosslinking between kappa casein molecules. In some cases, the kappa casein has an amino acid sequence comprising any one of SEQ ID NOs: 40-60, or a variant thereof comprising an amino acid sequence with at least 80% sequence identity to any one of SEQ ID NOs: 40-60. In some cases, the kappa casein comprises an amino acid sequence of cow, human, sheep, goat, buffalo, bison, horse, or camel kappa casein. In some cases, the micelle-like composition comprises a population of micelle-like structures with an average or mean size from about 300 nm to about 500 nm. In some cases, the micelle-like composition comprises a population of micelle-like structures with an average or mean size of about 100 nm to about 250 nm. In some cases, the micelle-like composition comprises a population of micelle-like structures with an average or mean size of about 600 nm to about 700 nm. In some cases, the micelle-like composition comprises a population of micelle-like structures with an average or mean size of about 400 nm. In some cases, the cross-linking agent is inactivated after formation of the micelle-like structures. In some cases, the cross-linking agent comprises transglutaminase. In some cases, the kappa casein has an amino acid sequence comprising any one of SEQ ID NOs: 40-60, or a variant thereof comprising an amino acid sequence with at least 80% sequence identity to any one of SEQ ID NOs: 40-60. In some cases, the kappa casein comprises an amino acid sequence of cow, human, sheep, goat, buffalo, bison, horse, or camel kappa casein protein. In some cases, the kappa casein has an amino acid sequence comprising any one of SEQ ID NOs: 43-45, or a variant thereof comprising an amino acid sequence with at least 80% sequence identity to any one of SEQ ID NOs: 43-45. In some cases, the kappa casein comprises an amino acid sequence of sheep kappa casein protein. In some cases, the kappa casein comprises a mixture of a native kappa casein and one or more altered forms thereof. In some cases, the one or more altered forms thereof is a truncated kappa casein (e.g., truncated relative to a native kappa casein). In some cases, the kappa casein comprises a first kappa casein protein and a second kappa casein protein. In some cases, the first kappa casein protein and the second kappa casein protein are from different mammalian species.

In yet another aspect, a dairy-like composition formed by the method of any one of the preceding methods is provided. In some cases, the dairy-like composition comprises a pasta filata cheese.

In some aspects, described herein are micelle compositions comprising an alpha casein and a kappa casein, wherein at least one of the alpha casein and the kappa casein is a recombinant protein, wherein the alpha casein, the kappa casein, or both the alpha casein and the kappa casein comprise a non-native post-translational modification feature, and wherein the alpha casein and the kappa casein are associated in micelles.

In some embodiments, a substantial portion of micelles of the micelle composition comprises intra-micellar crosslinking, and the majority of the micelles are not comprised within an inter-micellar crosslinked structure. In some embodiments, the non-native post-translational modification feature comprises a reduction in phosphorylation, a lack of phosphorylation, or an alteration of one or more sites of phosphorylation of the alpha casein.

In some embodiments, the non-native post-translational modification feature comprises a reduction in glycosylation, a lack of glycosylation, or an alteration of one or more sites of glycosylation of the kappa casein. In some embodiments, the non-native post-translational modification feature comprises a reduction in glycosylation, or a lack of glycosylation. In some embodiments, the alpha casein is a recombinant protein. In some embodiments, the kappa casein is a recombinant protein. In some embodiments, the alpha casein and the kappa casein are both recombinant proteins.

In some embodiments, the alpha casein, the kappa casein, or both the alpha casein and the kappa casein are produced in a microbial host cell. In some embodiments, the microbial host cell is a bacterium.

In some embodiments, the micelle composition further comprises a beta casein or a derivative thereof. In some embodiments, the micelle composition comprises a gamma casein. In some embodiments, the micelles do not comprise a beta casein or derivative thereof. In some embodiments, the ratio of alpha casein protein to kappa casein protein is from about 1:1 to about 15:1. In some embodiments, the ratio of alpha casein protein to kappa casein protein is about 1:1, 2:1, 3:1, 4:1, 5:1, 6:1, 7:1, 8:1, 9:1, 10:1, 11:1, 12:1, 13:1, 14:1, or 15:1. In some embodiments, the alpha casein protein is an alpha-S1 casein or an alpha-S2 casein. In some embodiments, the alpha casein protein has an amino acid sequence comprising any one of SEQ ID NOs: 1-39 or 64-72 or a variant thereof with at least 80% sequence identity. In some embodiments, the kappa casein protein has an amino acid sequence comprising one of SEQ ID NOs: 40-60 or a variant thereof with at least 80% sequence identity. In some embodiments, the micelle composition comprises a population of micelles with an average or mean size from about 200 nm to about 400 nm. In some embodiments, the micelle composition comprises a population of micelles with an average or mean size of about 300 nm.

In some embodiments, the micelle composition further comprises at least one salt selected from the group consisting of a calcium salt, a citrate salt, and a phosphate salt. In some embodiments, the micelle composition is susceptible to renneting.

In some aspects, described herein are micelle-like compositions comprising a kappa casein in the absence of alpha casein and beta casein, wherein the kappa casein forms a micelle-like structure. In some embodiments, the micelle-like compositions comprise intra-micellar crosslinking. In some embodiments, the kappa casein protein has an amino acid sequence comprising one of SEQ ID NOs: 40-60 or a variant thereof with at least 80% sequence identity. In some embodiments, the micelle-like composition comprises a population of micelle-like structures with an average or mean size from about 300 nm to about 500 nm. In some embodiments, the micelle-like composition comprises a population of micelle-like structures with an average or mean size of about 400 nm. In some embodiments, the micelle-like composition comprises a population of micelle-like structures with an average or mean size of about 100 nm to about 250 nm. In some embodiments, the micelle-like composition comprises a population of micelle-like structures with an average or mean size of about 600 nm to about 700 nm.

In some embodiments, the micelle-like composition further comprises at least one salt, selected from the group consisting of a calcium salt, a citrate salt, and a phosphate salt. In some embodiments, the micelle-like composition is susceptible to renneting.

In some aspects, described herein are dairy-like products comprising the micelle compositions or the micelle-like compositions. In some embodiments, the micelle-like composition does not include any additional dairy-derived protein. In some embodiments, the dairy-like product is incorporated into an edible composition, wherein the edible composition does not include any animal-obtained dairy protein. In some embodiments, the dairy-like product is selected from the group consisting of milk, yogurt, curd, cheese, cream, and ice cream. In some embodiments, the dairy-like product comprises a cheese selected from the group consisting of a soft cheese, a hard cheese a pasta filata cheese, or an aged cheese. In some embodiments, the cheese has a fat content from about 0% to about 50% and the fat is derived from a plant-based source. In some embodiments, the cheese has a sugar content from about 0% to about 10% and the sugar is derived from a plant-based source. In some embodiments, the cheese is capable of melting and browning when heated. In some embodiments, the cheese is selected from the group consisting of pasta filata like cheese, paneer, cream cheese, and cottage cheese. In some embodiments, the cheese is an aged or matured cheese selected from the group consisting of cheddar, Swiss, and gouda. In some embodiments, the cheese is mozzarella. In some embodiments, the moisture retention of the cheese is from about 40% to about 65%. In some embodiments, the texture of the cheese is comparable to an animal-obtained dairy cheese. In some embodiments, the hardness of the cheese is comparable to an animal-obtained dairy cheese.

In some aspects, described herein are powders comprising the micelle composition or the micelle-like composition. In some embodiments, the casein content of the powder is from about 50% to about 90%.

In some aspects, described herein are methods of making a dairy-like ingredient comprising providing an alpha casein and a kappa casein, wherein the alpha casein, the kappa casein, or both the alpha casein and the kappa casein comprise a non-native post-translational modification feature; inducing micelle formation; and providing a crosslinking agent under conditions for inducing intra-micellar crosslinking; wherein the method produces micelles comprising the alpha casein and the kappa casein in a form suitable for a dairy-like ingredient.

In some aspects, described herein are methods of making a dairy-like ingredient comprising providing an alpha casein, wherein the alpha casein comprises a non-native post-translational modification feature; providing a crosslinking agent under conditions for crosslinking the alpha casein; and mixing kappa casein with the crosslinked alpha casein under conditions to induce micelle formation; wherein the method produces micelles comprising the alpha casein and the kappa casein in a form suitable for a dairy-like ingredient. In some embodiments, the alpha casein and kappa casein are incubated together prior to adding the crosslinking agent. In some embodiments, the crosslinking agent is added from about 30 minutes and about 24 hours after the alpha casein and kappa casein are incubated together. In some embodiments, the crosslinking agent is added from about 1 hour and about 12 hours after the alpha casein and kappa casein are incubated together. In some embodiments, the crosslinking agent is added prior to the step of inducing micelle formation. In some embodiments, the crosslinking agent is added subsequent to the step of inducing micelle formation. In some embodiments, the crosslinking agent is a transglutaminase.

In some embodiments, the non-native post-translational modification feature comprises a reduction in phosphorylation, a lack of phosphorylation, or an alteration of one or more sites of phosphorylation on the alpha casein. In some embodiments, the non-native post-translational modification feature comprises a reduction in glycosylation, a lack of glycosylation, or an alteration of one or more sites of glycosylation on the kappa casein. In some embodiments, the method further comprises producing the alpha casein, the kappa casein, or both in a recombinant host cell. In some embodiments, the recombinant host cell is a microbial cell. In some embodiments, the microbial cell is selected from the group consisting of Lactococci sp., Lactococcus lactis, Bacillus subtilis, Bacillus amyloliquefaciens, Bacillus lichenformis, Bacillus megaterium, Mycobacterium smegmatis, Rhodococcus erythropolis, Corynebacterium glutamicum, Lactobacilli sp., Lactobacillus fermentum, Lactobacillus casei, Lactobacillus acidophilus, Lactobacillus plantarum, Synechocystis sp. 6803, and Escherichia coli. In some embodiments, the dairy-like ingredient is susceptible to renneting.

In some embodiments, the conditions to induce micelle formation include the addition of a salt. In some embodiments, the micelles are comprised in a liquid colloid. In some embodiments, the methods further comprise the step of forming a dairy-like product with the dairy-like ingredient. In some embodiments, the dairy-like product comprises milk, cream, curd, cheese, yogurt, or ice cream. In some embodiments, the methods further comprise subjecting the liquid colloid to a first condition to form coagulates. In some embodiments, the first condition is the addition of acid or acidification of the liquid colloid with a microorganism.

In some embodiments, the method further comprises subjecting the coagulates to a hot water treatment and optionally stretching to form a filata-type cheese. In some embodiments, the method further comprises subjecting the coagulates to a renneting agent to form a rennetted curd. In some embodiments, the renneting agent is a microbially-derived chymosin enzyme. In some embodiments, the method further comprises aging and maturing the rennetted curd to form an aged or matured cheese-like composition. In some embodiments, the method further comprises subjecting the rennetted curd to a hot water treatment and optionally stretching to form a filata-type cheese.

In some embodiments, the methods further comprise forming a yogurt from the liquid colloid. In some embodiments, forming the yogurt comprises heating and then cooling the liquid colloid, and acidifying the liquid colloid with a microorganism. In some embodiments, the microorganism comprises one or more of Lactobacillus delbrueckii subsp. Bulgaricus, Streptococcus thermophilus, a Lactobacilli, or a Bifidobacteria species. In some embodiments, the micelles do not include beta casein protein. In some embodiments, the dairy-like ingredient does not include any additional dairy-derived protein. In some embodiments, the dairy-like ingredient does not include any animal-obtained dairy protein. In some embodiments, the dairy-like product comprises a fat, a sugar, a flavoring, or a colorant. In some embodiments, the dairy-like ingredient is in a powder form. In some embodiments, the method further comprises drying, lyophilizing, or spray-drying to produce the powder form. In some embodiments, the ratio of alpha casein protein to kappa casein protein is from about 1:1 to about 15:1. In some embodiments, the ratio of alpha casein protein to kappa casein protein is about 1:1, 2:1, 3:1, 4:1, 5:1, 6:1, 7:1, 8:1, 9:1, 10:1, 11:1, 12:1, 13:1, 14:1, or 15:1.

In some embodiments, the alpha casein protein is an alpha-S1 casein or an alpha-S2 casein. In some embodiments, the alpha casein protein has an amino acid sequence comprising any one of SEQ ID NOs: 1-39 or 64-72 or a variant thereof with at least 80% sequence identity. In some embodiments, the alpha casein protein comprises an amino acid sequence of cow, human, sheep, goat, buffalo, bison, horse, or camel alpha casein protein. In some embodiments, the kappa casein protein comprises an amino acid sequence of cow, human, sheep, goat, buffalo, bison, horse, or camel kappa casein protein. In some embodiments, the kappa casein protein has an amino acid sequence comprising any one of SEQ ID NOs: 40-60 or a variant thereof with at least 80% sequence identity.

In some aspects, described herein are coagulated compositions formed by any of the methods provided herein. In some aspects, described herein are renneted curd compositions formed by any of the methods provided herein. In some aspects, described herein are dairy-like compositions formed by any of the methods provided herein.

In some embodiments, the dairy-like composition is selected from the group consisting of milk, cream, curd, cheese, yogurt, and ice cream. In some embodiments, the dairy-like composition is selected from the group consisting of pasta-filata like cheese, paneer, cream cheese, cottage cheese, cheddar, Swiss, gouda, and mozzarella.

In some aspects, described herein are micelle compositions comprising an alpha casein and a kappa casein, wherein at least one of the alpha casein and the kappa casein is a recombinant protein, wherein the alpha casein, the kappa casein, or both the alpha casein and the kappa casein comprise a non-native post-translational modification feature, and wherein a substantial portion of micelles of the micelle composition comprises cross-linked casein protein. In some embodiments, a substantial portion of the micelles of the micelle composition comprises intra-micellar crosslinking, and the majority of the micelles of the micelle composition are not comprised within an inter-micellar crosslinked structure. In some embodiments, a substantial portion of the alpha casein and the kappa casein are comprised in micellar form.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings.

FIG. 1 illustrates average micelle diameter (in nm) of casein micelles, as well as any submicelles or larger aggregates present in liquid colloid formed using hypophosphorylated alpha casein treated with TG (transglutaminase) followed by addition of kappa casein and salts (1), mixture of hypophosphorylated alpha casein and kappa casein treated with TG followed by addition of salts (calcium, phosphate, and citrate) (3), overnight incubated mixture of hypophosphorylated alpha casein and kappa casein treated with TG followed by addition of salt (5), and salt addition to the mixture of hypophosphorylated alpha casein and kappa casein followed by TG treatment (7). Bottom row samples 2, 4, 6, and 8 are same as top row samples 1, 3, 5, and 7 respectively except the samples are not treated with TG, but incubated in the same conditions (1-hour incubation at 40° C. step followed by 10 minutes at 78° C. inactivation, in addition to otherwise room temperature incubation steps). Relative intensity proportions of different micellar/particle peaks detected are represented as arch sizes/angles of black and grey arches.

FIG. 2 shows Tris-Glycine native-PAGE analysis (non-reducing) of hypophosphorylated alpha casein and kappa casein under salts (calcium, phosphate, and citrate) and various protein order addition conditions, with and without transglutaminase, as per FIG. 1, samples 1-8. Samples not treated with transglutaminase were incubated in the same conditions as transglutaminase-treated samples (1-hour incubation at 40° C. step followed by 10 minutes at 78° C. inactivation, in addition to otherwise room temperature incubation steps). Samples x and y are control samples of only hypophosphorylated alpha casein by itself and a mixture of hypophosphorylated alpha casein and kappa casein respectively.

FIG. 3 shows wet yields (in grams (g) of cheese per g of protein) made from curds using hypophosphorylated alpha casein and kappa casein micelles induced under salts (calcium, phosphate, and citrate) and various protein order addition conditions, with and without transglutaminase (denoted as TG), as per FIG. 1, samples 1-8. Samples 9 and 10 are control samples from an independent triplicate experiment for skim milk (+/−0.18 stdv on triplicate) and micellar casein (+/−0.30 stdv on triplicate). Cheese (mozzarella) was made by dipping the curd in hot water, stretching, and shaping to a cheese ball.

FIG. 4 illustrates average micelle diameter (in nanometers (nm)) of casein micelles, as well as any submicelles or larger aggregates present in liquid colloid formed using alpha, beta, and kappa casein (1 and 2), alpha and kappa casein only (3 and 4), or hypophosphorylated alpha and kappa casein only (5 and 6), with and without transglutaminase (TG) treatment after micellar induction using salts (calcium, phosphate, and citrate). Samples 1, 3 and 5 are treated with TG. Sample 2, 4, and 6 are control samples not treated with TG but incubated in same conditions (1-hour incubation at 40° C. step followed by 10 minutes at 78° C. inactivation, in addition to otherwise room temperature incubation steps). Relative intensity proportions of different micellar/particle peaks detected are represented as arch sizes/angles of black and grey arches.

FIG. 5 shows Tris-Glycine native-PAGE analysis (non-reducing) of alpha, beta, and kappa casein (1 and 2), alpha and kappa casein only (3 and 4), or hypophosphorylated alpha and kappa casein only (5 and 6), with and without transglutaminase (TG) treatment after micellar induction with salts (calcium, phosphate, and citrate), as per FIG. 4, samples 1-6. Samples 1, 3 and 5 are treated with TG. Sample 2, 4, and 6 are control samples not treated with TG but incubated in same conditions (1-hour incubation at 40° C. step followed by 10 minutes at 78° C. inactivation, in addition to otherwise room temperature incubation steps).

FIG. 6 shows wet yields (in g of cheese per g of protein) made from curds using alpha and kappa casein (1), hypophosphorylated alpha casein and kappa casein (2 and 3) micelles induced with salts (calcium, phosphate, and citrate), where hypophosphorylated alpha casein and kappa casein micelles were treated for 15 minute (2) or 30 minutes (3) with transglutaminase (TG). Cheese (mozzarella) was made by dipping the curd in hot water, stretching, and shaping to a cheese ball.

FIG. 7 shows hardness (in g force applied) of cheese made from curds using alpha and kappa casein (1), hypophosphorylated alpha casein and kappa casein (2 and 3) micelles induced with salts (calcium, phosphate, and citrate), where hypophosphorylated alpha casein and kappa casein micelles were treated for 15 minutes (2) or 30 minutes (3) with transglutaminase (TG). Cheese (mozzarella) was made by dipping the curd in hot water, stretching, and shaping to a cheese ball. Measurement was performed on a texture analyzer using a stress-relaxation test.

FIG. 8 illustrates micelle and sub-micelle particle size distributions in log 10 (nm) for Transglutaminase (TG) treated native bovine alpha casein+native bovine kappa casein (1), untreated native bovine alpha casein+native bovine kappa casein (2), TG treated hypophosphorylated alpha casein+native bovine kappa casein (3), untreated hypophosphorylated alpha casein+native bovine kappa casein (4), TG treated recombinant bovine alpha-S1-casein+native bovine kappa casein (5), untreated recombinant bovine alpha-S1-casein+native bovine kappa casein (6), TG treated recombinant bovine alpha-S1-casein+recombinant sheep kappa casein (7), and untreated recombinant bovine alpha-S1-casein+recombinant sheep kappa casein (8). Each sample is measured thrice. The dot in the plot represents a particle population of a particular size, and its color intensity corresponds to the proportion of that particle population among all particle populations detected.

FIG. 9 shows wet yields (in g of cheese per g of protein) made from curds using alpha and kappa casein (1), hypophosphorylated alpha casein and bovine kappa casein (2), recombinantly made alpha-S1-casein and bovine kappa casein (3), and recombinantly made bovine alphaS1 and recombinantly made sheep kappa casein (4), micelles induced with salts (calcium, phosphate, and citrate), where micelles were treated for 30 minutes with transglutaminase (TG). Cheese (mozzarella) was made by dipping the curd in hot water, stretching, and shaping to a cheese ball.

FIG. 10 illustrates average particle diameter (in nm) of micelle-like structures, as well as any submicelle-like structures or larger aggregates present in liquid colloid formed using only kappa casein by itself, with and without transglutaminase treatment after micellar induction using salts (calcium, phosphate, and citrate). Sample 1 is treated with transglutaminase. Sample 2 is control sample not treated with transglutaminase but incubated in same conditions (1-hour incubation at 40° C. step followed by 10 minutes at 78° C. inactivation, in addition to otherwise room temperature incubation steps). Relative intensity proportions of different micellar/particle peaks detected are represented as arch sizes/angles of black and grey arches.

FIG. 11 shows Tris-Glycine native-PAGE analysis (non-reducing) of alpha casein only (1 and 2), beta casein only (3 and 4), and kappa casein only (5 and 6), with and without transglutaminase (TG) treatment after micellar induction with salts (calcium, phosphate, citrate). Samples 1, 3 and 5 are TG treated. Samples not treated with TG (2, 4 and 6) were incubated in same conditions as transglutaminase-treated samples (1-hour incubation at 40° C. step followed by 10 minutes at 78° C. inactivation, in addition to otherwise room temperature incubation steps).

FIG. 12 shows wet yields (in g of cheese per g of protein) of cheese-like product made using alpha casein only (1, 2) beta casein only (3, 4) and kappa casein only (5, 6), with and without transglutaminase (TG) treatment after micellar induction with salts (calcium, phosphate, citrate), as per FIG. 11. Only kappa casein samples gave curd, whereas alpha and beta casein samples gave just protein aggregates. Cheese (mozzarella) was made by dipping the curd (kappa) or precipitated protein aggregates (alpha, beta) in hot water, stretching, and shaping to a cheese ball.

FIG. 13 illustrates micelle and sub-micelle particle size distributions in log 10 (nm) for transglutaminase (TG) treated and untreated native bovine kappa casein. Sample 1 is TG treated and sample 2 is untreated but incubated in same conditions as transglutaminase-treated samples (30-minute incubation at 40° C. step). Each sample is measured thrice. The dot in the plot represents a particle population of a particular size, and its color intensity corresponds to the proportion of that particle population among all particle populations detected.

FIG. 14 shows wet yields (in g of cheese per g of protein) made from curds using bovine kappa casein), where micelles were treated for 30 minutes with transglutaminase (TG). Cheese (mozzarella) was made by dipping the curd in hot water, stretching, and shaping to a cheese ball.

FIG. 15 illustrates micelle and sub-micelle particle size distributions in log 10 (nm) for transglutaminase (TG) treated and untreated native bovine kappa casein (1 and 2), and TG treated and untreated recombinantly made sheep kappa casein (3 and 4). Sample 1 and 3 are TG treated and sample 2 and 4 are untreated but incubated in the same conditions as transglutaminase-treated samples (30-minute incubation at 40° C. step). Each sample is measured thrice. The dot in the plot represents a particle population of a particular size, and its color intensity corresponds to the proportion of that particle population among all particle populations detected.

### DETAILED DESCRIPTION

Although the dairy industry is worth $330 billion, research needs to be performed for a clean dairy solution using recombinant dairy proteins. In terms of resources needed per gram many dairy products are inefficient to produce. It is also difficult to accurately reproduce dairy-like products from plant-based ingredients alone. Presented herein are micelle compositions and micelle-like compositions, methods of making micelle and micelle-like compositions, and methods of making dairy-like products from such compositions.

A component that gives dairy products, such as cheese or yogurt, their unique characteristics is the casein proteins that form micelles in milk. Micelles are protein colloids comprised of casein proteins (e.g., alpha-S1 casein, alpha-S2 casein, beta casein, kappa casein, and a cleaved form of beta casein called gamma casein) that interact with insoluble calcium phosphate at the colloid center. It is the micelles in milk that attract each other once chymosin is added to milk. This forms the curd, which is then used to make 99% of all cheeses. In the case of yogurt, acidification of the micelles comprised in the liquid colloid may be performed using a starter culture of bacteria known for yogurt production.

The following disclosure is partly based on the surprising observation that kappa casein can independently form micelle-like structures or micelle-like particles (e.g., without alpha and/or beta casein) which do not aggregate or polymerize, and which, when formed into a dairy-like product (e.g., milk-like liquid colloid or cheese), results in a stretchy and melty cheese of desirable properties. The following disclosure is also partly based on the surprising discovery that recombinantly made kappa casein lacking PTMs can form stable micelles and make dairy-like curd using transglutaminase-induced crosslinking for stabilization, while not introducing polymerization or crosslinking that inhibits renneting (e.g., across the C-terminal part of the protein), resulting in stable curd that makes cheese that stretches and melts better than dairy cheeses (e.g., mozzarella). The following disclosure is also based on the surprising observation that adding a crosslinking agent (e.g., transglutaminase) after micelle formation (e.g., formed from either alpha and kappa casein, or from kappa casein alone) improves yield and properties of the resulting cheese-like product.

An aspect of the present disclosure is directed to micelle compositions composed of one or more recombinant casein proteins. The micelle compositions can include caseins as described herein (e.g., alpha, beta, kappa, and/or gamma caseins). In some cases, the micelle compositions may include alpha, beta, kappa, and/or gamma casein (e.g., alpha casein and kappa casein). In some embodiments, the recombinant alpha casein, kappa casein, or both the alpha casein and the kappa casein may include a non-native post-translational modification (PTM) feature.

As provided herein, the non-native PTM feature may include a reduction in phosphorylation, a lack of phosphorylation, or an alteration of one or more sites of phosphorylation of a casein (e.g., a recombinant alpha casein that is reduced or altered in phosphorylation as compared to native alpha casein). Micelles formed with hypophosphorylated or non-phosphorylated caseins can be larger than micelles formed with native caseins. The non-native PTM feature may include a reduction in glycosylation, a lack of glycosylation, or an alteration of one or more sites of glycosylation of a casein (e.g., a recombinant kappa casein that is reduced or altered in glycosylation as compared to native kappa casein).

In some embodiments, all or at least a portion (e.g., a substantial portion) of the micelles in the micelle composition may include intra-micellar crosslinking, i.e., crosslinks formed between the caseins within a micelle. In various cases, a majority of the micelles in the micelle composition may not be included within an inter-micellar crosslinking structure (i.e., crosslinking of individual micelles to each other). The crosslinking within micelles can improve the stability, modify the size, and/or modify other features of micelles and dairy-like products made from the micelles.

Hypophosphorylated or non-phosphorylated caseins can result in the production of micelles that form dairy-like products (e.g., cheese-like products) of desired qualities, such as desired melt, stretch, or yield. Intra-micellar crosslinking of micelles containing hypophosphorylated or non-phosphorylated caseins may result in improved properties such as improvements in efficiency of micelle formation, stability (such as for homogenization), and liquid colloid formation. Intra-micellar crosslinking of micelles containing hypophosphorylated or non-phosphorylated caseins may result in improved yield for dairy like products and improved texture and hardness that mimics dairy products produced from animal-obtained casein micelles.

Another aspect of the present disclosure is directed to micelle-like compositions. The micelle-like composition may include a single type of casein (e.g., kappa casein). In various cases, the micelle-like composition may include kappa casein and lack alpha casein and beta casein. In various cases, the micelle-like composition may include kappa casein and may not comprise crosslinking. In other aspects, the micelle-like composition may include kappa casein and may comprise crosslinking (e.g., inter-molecular crosslinking between kappa casein molecules within the micelle-like structures). The single type of casein (e.g., kappa casein) may form a micelle-like composition and may include native and/or non-native crosslinking between the casein proteins within the micelle-like structure. In some embodiments, the kappa casein may be a native (e.g., full-length) kappa casein protein. In some embodiments, the kappa casein may be a truncated kappa casein protein (e.g., truncated relative to a native (e.g., full-length) kappa casein protein. In some cases, the truncated kappa casein protein may comprise an N-terminal truncation (e.g., relative to a native (e.g., full-length) kappa casein protein. In some cases, the truncated kappa casein protein may comprise a C-terminal truncation (e.g., relative to a native (e.g., full-length) kappa casein protein. In some cases, the truncated kappa casein protein may comprise both an N-terminal truncation and a C-terminal truncation (e.g., relative to a native (e.g., full-length) kappa casein protein. In some embodiments, the kappa casein may comprise a mixture of a native (e.g., full-length) kappa casein protein and an altered form thereof (e.g., a truncated kappa casein protein). In some embodiments, the micelle-like compositions may comprise a first kappa casein protein and a second kappa casein protein, wherein the first kappa casein protein and the second kappa casein protein are from different mammalian species. Micelle compositions and micelle-like compositions as provided herein can produce dairy-like products with properties that mimic dairy products produced from animal-obtained micelles.

Another aspect of the present disclosure is directed to dairy-like products that include the micelle compositions as described herein. The dairy-like products may include the micelle-like compositions as described herein. As discussed in further detail herein, the dairy-like products may include milk, milk-like products, yogurt, yogurt-like products, curd, curd-like products, cheese, cheese-like products, cream, cream-like products, ice cream, ice cream-like products, or other suitable dairy-like products.

Another aspect of the present disclosure is directed to methods of making dairy-like ingredients. The dairy-like ingredients may include micelle compositions as provided herein. The dairy-like ingredients may include micelle-like compositions as provided herein.

The methods of making the dairy-like ingredient may include obtaining or providing one or more recombinant caseins as discussed herein. In some embodiments, the recombinant caseins may include alpha, beta, kappa, and/or gamma casein (e.g., alpha casein and a kappa casein), wherein the recombinant caseins lack or include post-translational modifications. Other combinations of caseins are also within the scope of this disclosure. In embodiments, the micelles contain recombinant alpha and kappa casein proteins and the recombinant alpha casein, the recombinant kappa casein, or both the recombinant alpha casein and the recombinant kappa casein may include a non-native PTM feature. The methods of making the dairy-like ingredient may include inducing micelle formation. Furthermore, the methods of making the dairy-like ingredient may include adding or providing a crosslinking agent (e.g., transglutaminase) to the micelles to induce intra-micellar crosslinking. In certain cases, the methods of making the dairy-like ingredient may produce micelles comprising the alpha, beta, kappa, and/or gamma casein (e.g., the alpha casein and the kappa casein) in a form suitable for a dairy-like ingredient.

Adding a crosslinking agent to the one or more caseins before, in parallel with, or after the induction of micelle production can improve micelle formation in comparison to methods where a crosslinking agent is not added. Furthermore, adding a crosslinking agent after the production of micelles is induced can produce compositions where more of the casein is irreversibly micellar and/or where monomeric casein is depleted. This can improve efficiency by decreasing the amount of casein that is needed to make a dairy-like ingredient, improve the yields for production of dairy-like ingredients and products and improve the qualities of the ingredients and products.

Intra-micellar crosslinking of micelles formed with one or more recombinant casein proteins with non-native PTMs may be improved as compared to crosslinking of micelles with native caseins (such as those found in animal milk or formed with caseins derived from animal milk). For example, use of hypophosphorylated or non-phosphorylated alpha casein protein can improve incorporation of casein in crosslinked micelles as compared to micelles formed from alpha casein with a phosphorylation comparable to native casein. Furthermore, adding a crosslinking agent (e.g., transglutaminase) can result in crosslinks in intra-micellar positions instead of in crosslinks on the outside of a micelle and/or across different micelles (i.e., inter-micellar crosslinking). Inter-micellar crosslinking may interfere with dairy product production, such as by reducing renneting efficiency in cheese-making.

In some other embodiments, the methods of making a dairy-like ingredient may include obtaining or providing a recombinant alpha casein. The recombinant alpha casein may include one or more non-native PTM features. The methods of making the dairy-like ingredient may include adding or providing a crosslinking agent (e.g., transglutaminase) to the alpha casein under conditions for crosslinking the alpha casein. Furthermore, the methods of making the dairy-like ingredient may include mixing kappa casein (e.g., with or without non-native PTM features) with the crosslinked alpha casein to induce micelle formation. The methods of making the dairy-like ingredient may produce micelles comprising the alpha casein and the kappa casein in a form suitable for the dairy-like ingredient. The dairy-like ingredient may be combined with one or more other ingredients to form a dairy-like product as discussed herein. In some embodiments, a beta casein protein may be included in the micelles. In some embodiments, a beta casein protein is not included in the micelles.

The term “about,” as used herein, can mean within 1 or more than 1 standard deviation. Alternatively, “about” can mean a range of up to 10%, up to 5%, or up to 1% of a given value. For example, “about” can mean up to ±10%, ±9%, ±8%, ±7%, ±6%, ±5%, ±4%, ±3%, ±2%, or 1% of a given value.

The term “dairy protein,” as used herein, generally refers to a protein that has an amino acid sequence derived from a protein found in milk (including variants thereof).

The term “animal-obtained,” as used herein in reference to a dairy protein, generally refers to a protein obtained or derived from milk or a milk source (such as a caseinate made from milk), or such as a protein obtained and/or isolated and/or derived from a milk-producing organism, including, but not limited to, cow, sheep, goat, human, bison, buffalo, camel, and horse. “Animal-obtained casein protein”, as used herein, generally refers to casein protein obtained and/or isolated and/or derived from a milk-producing organism.

The term “recombinant dairy protein,” as used herein, generally refers to a protein that is expressed in a heterologous or recombinant organism that has an amino acid sequence derived from a protein found in milk (including variants thereof). “Recombinant casein protein,” as used herein, generally refers to a casein produced by a recombinant organism or in a heterologous host cell.

The term “liquid colloid,” as used herein, generally refers to a liquid comprising micelles, where the micelles are substantially in suspension within the liquid. Stated another way, the micelles can remain dispersed and do not settle out of the liquid solution. In some cases, the liquid colloid can include casein containing micelles and other forms of the caseins such as aggregates that do settle out and/or monomeric forms of the caseins.

A percentage of “sequence identity,” as used herein in the context of polynucleotide or polypeptide (amino acid) sequences, generally refers to the percentage of residues in two sequences that are the same when the sequences are aligned for maximum correspondence. There are a number of different algorithms that can be used to measure polynucleotide or polypeptide sequence identity. For instance, sequences can be compared using FASTA (e.g., using its default parameters as provided in the Wisconsin Package Version 10.0, Genetics Computer Group (GCG), Madison, WI), Gap (e.g., using its default parameters as provided in the Wisconsin Package Version 10.0, GCG, Madison, WI), Bestfit, ClustalW (e.g., using default parameters of Version 1.83), or BLAST (e.g., using reciprocal BLAST, PSI-BLAST, BLASTP, BLASTN) (see, for example, Pearson. 1990. Methods Enzymol. 183:63; Altschul et al. 1990. J. Mol. Biol. 215:403).

While various embodiments have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments described herein may be employed.

### I. Micelle and Micelle-Like Compositions

In mammalian milk, casein proteins (alpha-S1 casein, alpha-S2 casein, beta casein, kappa casein, and gamma casein), calcium phosphate, and citrate form colloidal particles called casein micelles. Casein proteins can be generated recombinantly and such recombinant caseins can be used to form micelles. A micelle composition, as provided herein, can include a plurality of micelles or a population of micelles. The micelles of the micelle composition can include one or more types of caseins (e.g., alpha casein, beta casein, kappa casein, or gamma casein). At least a portion of the caseins can include one or more non-native post-translational modification (PTM) features as described herein. Surprisingly, a substantial portion of the micelles in the micelle composition can include cross-linking between casein proteins within each micelle (intra-micellar crosslinking) that improves the performance of micelles formed from caseins with a non-native PTM. The micelle compositions herein differ from the structures created when casein proteins are polymerized, such as by including a crosslinking agent. In such polymerization, the casein proteins form long strings of linked casein molecules. However, such polymers are not micelles or micelle-like forms and are generally not suitable for dairy applications that utilize micelles or micelle-like forms (e.g., cheese making). Furthermore, in the compositions herein, a majority of the micelles of the micelle composition may not be comprised within inter-micellar crosslinked structures.

The level of inter-micellar crosslinking can be estimated by measuring large polymers and/or irreversible aggregates that are formed in a liquid colloid including micelles. In some embodiments, none or substantially none of the micelles in the liquid colloid are in or part of large polymers and/or aggregates. In some embodiments, no more than about 0.5%, no more than about 1%, no more than about 2%, no more than about 3%, no more than about 4%, no more than about 5%, no more than about 10%, no more than about 15%, no more than about 20%, no more than about 25%, no more than about 30% of the micelles in the liquid colloid may be in or part of large polymers and/or aggregates. In certain embodiments, from 2.5% to 35%, 5% to 30%, 10% to 25%, or 15% to 20% of the micelles in the liquid colloid may be in or part of large polymers and/or aggregates. The level of inter-micellar crosslinking can also be estimated by measuring gelling or other suitable properties of the micelle composition.

As described herein, casein micelles may be formed with isolated casein proteins, such as recombinantly produced casein proteins. A combination of such micelles may form a micelle composition. Micelles formed from recombinant casein may include at least one of alpha casein, such as alpha-S1 casein and/or alpha-S2 casein; beta casein; kappa casein; or gamma casein. In some cases, micelles may comprise alpha casein and kappa casein. In certain cases, micelles may comprise alpha casein and kappa casein, and may not contain any beta casein protein or a derivative thereof. The alpha casein, the kappa casein, or both casein proteins may be recombinant proteins such that the alpha casein, the kappa casein, or both have a non-native PTM. In some cases, the alpha casein, the kappa casein, or both casein proteins are recombinant proteins with non-native PTMs. The alpha casein, the kappa casein, or both casein proteins may be recombinant proteins such that the alpha casein, the kappa casein, or both caseins lack PTMs. In certain cases, the alpha casein, the kappa casein, or both caseins are recombinant proteins that lack PTMs. In various cases, micelles may include beta casein or a derivative thereof. In some cases, micelles may include gamma casein. In some embodiments, the kappa casein may be a native (e.g., full-length) kappa casein protein. In some embodiments, the kappa casein may be a truncated kappa casein protein (e.g., truncated relative to a native (e.g., full-length) kappa casein protein. In some cases, the truncated kappa casein protein may comprise an N-terminal truncation (e.g., relative to a native (e.g., full-length) kappa casein protein. In some cases, the truncated kappa casein protein may comprise a C-terminal truncation (e.g., relative to a native (e.g., full-length) kappa casein protein. In some cases, the truncated kappa casein protein may comprise both an N-terminal truncation and a C-terminal truncation (e.g., relative to a native (e.g., full-length) kappa casein protein. In some embodiments, the kappa casein may comprise a mixture of a native (e.g., full-length) kappa casein protein and an altered form thereof (e.g., a truncated kappa casein protein). In some embodiments, the alpha casein may be a native (e.g., full-length) alpha casein protein. In some embodiments, the alpha casein may be a truncated alpha casein protein (e.g., truncated relative to a native (e.g., full-length) alpha casein protein. In some cases, the truncated alpha casein protein may comprise an N-terminal truncation (e.g., relative to a native (e.g., full-length) alpha casein protein. In some cases, the truncated alpha casein protein may comprise a C-terminal truncation (e.g., relative to a native (e.g., full-length) alpha casein protein. In some cases, the truncated alpha casein protein may comprise both an N-terminal truncation and a C-terminal truncation (e.g., relative to a native (e.g., full-length) alpha casein protein. In some embodiments, the alpha casein may comprise a mixture of a native (e.g., full-length) alpha casein protein and an altered form thereof (e.g., a truncated alpha casein protein). In some embodiments, the micelle compositions may comprise kappa casein protein (e.g., native, truncated, or a mixture of native and truncated) and alpha casein protein (e.g., native, truncated, or a mixture of native and truncated), and the kappa casein and the alpha casein may be from different mammalian species. In a particular embodiment, the alpha casein may comprise an amino acid sequence from bovine alpha casein, and the kappa casein may comprise an amino acid sequence from ovine alpha casein.

A micelle-like composition as provided herein can include a plurality of micelle-like structures or a population of micelle-like structures. The micelle-like structures of the micelle-like composition may include a kappa casein in the absence of alpha casein and beta casein. In some embodiments, the kappa casein can form a micelle-like structure. The kappa casein may include non-native inter-molecular crosslinking between kappa casein molecules within the micelle-like structures. The kappa casein may be crosslinked via an external crosslinking agent within the micelle-like structures. In certain instances, at least 5%, at least 10%, at least 15%, at least 20%, at least 25%, at least 30%, at least 35%, at least 40%, at least 45%, at least 50%, at least 55%, at least 60%, at least 65%, at least 70%, or greater than 70% of the kappa caseins may be crosslinked with one or more kappa caseins within the micelle-like structures via an external crosslinking agent. In various instances, from 2.5% to 35%, 5% to 30%, 10% to 25%, 15% to 20%, 10% to 70%, 20% to 60%, or 30% to 50% of the kappa caseins may be inter-molecularly and/or intra-molecularly crosslinked with one or more kappa caseins within the micelle-like structures via an external crosslinking agent.

The micelle composition or the micelle-like composition may be susceptible to renneting. That is, following acidification of the micelle composition or the micelle-like composition, a renneting agent may be added to form a renneted curd (coagulated curd matrix), which may then be used to make any type of cheese. Micelles in a micelle composition or micelle-like structures in a micelle-like composition, as described herein, can be stable and repel each other in colloidal suspension. In the presence of renneting agents or milk-clotting enzymes, and when acidified, micelles or micelle-like structures can be destabilized and attract each other, and thus coagulate. In the presence of renneting agents or milk-clotting enzymes, crosslinked coagulated curd matrix can be formed.

In some cases, the micelle composition or the micelle-like composition may include at least one salt. For example, the micelle composition or the micelle-like composition may include a calcium salt, a citrate salt, a phosphate salt, or a combination thereof.

In some embodiments, micelles described herein can include micelles formed in a liquid solution. In certain embodiments, casein-containing micelles may be present in a liquid colloid, where the micelles remain dispersed and do not settle out of the liquid solution. In various cases, the liquid colloid can include casein-containing micelles and other forms of the caseins such as aggregates and/or monomeric forms of the proteins. In some embodiments, the crosslinked micelle compositions described herein are improved in the formation of liquid colloid, such that the proportion of micelles that remain suspended in the liquid is increased as compared to micelles without crosslinking, and such that the proportion of casein in micellar forms is increased and the proportion of casein that is solubilized and monomeric is decreased as compared to micelles without crosslinking.

### Alpha Casein

In some embodiments, a micelle composition herein may comprise alpha casein protein. The alpha casein in the micelle composition may be alpha-S1 casein. The alpha casein in the micelle composition may be alpha-S2 casein. The alpha casein in the micelle composition may be a combination of alpha-S1 and alpha-S2 caseins. The total proportion of alpha casein in the micelle composition may comprise from 5% to 95% of the casein in the micelle composition. In some cases, the alpha casein may comprise at least 10%, at least 20%, at least 30%, at least 40%, at least 50%, at least 60%, at least 70%, at least 80%, at least 90%, or at least 95% of the casein in the micelle composition.

In some cases, the alpha casein in the micelle compositions herein may comprise from 0% alpha-S1 casein to 100% alpha-S1 casein. In certain cases, the remainder of the total proportion of alpha casein in the micelle composition may be alpha-S2 casein. In various cases, alpha-S1 casein is the only alpha casein (e.g., no alpha-S2 casein) in the micelle composition. In some embodiments, the alpha casein in the micelle composition is alpha-S2 casein. In some cases, alpha casein in a micelle composition may comprise from 0% alpha-S2 casein to 100% alpha-S2 casein. In various cases, the remainder of the total proportion of alpha casein in the micelle composition may be alpha-S1 casein. In some cases, alpha-S2 casein is the only alpha casein (e.g., no alpha-S1 casein) in the micelle composition.

In certain embodiments, the alpha casein in a micelle composition can be a mixture of alpha-S1 casein and alpha-S2 casein. The alpha casein in such a micelle composition may comprise, for example, from 1% alpha-S2 casein to 99% alpha-S2 casein and from 99% alpha-S1 casein to 1% alpha-S1 casein, respectively. In various embodiments, the alpha casein in the micelle composition may be a mixture of alpha-S1 casein and alpha-S2 casein in ratio of 10:90, 20:80, 30:70, 40:60, 50:50, 60:40, 70:30, 80:20, or 90:10. In some cases, the alpha casein protein in a micelle composition does not include alpha-S2 casein. In certain cases, the alpha casein protein in a micelle composition does not include alpha-S1 casein.

The protein content of a micelle composition herein may comprise from 30% to 90%, or 50% to 95%, alpha casein protein. In some cases, the protein content of a micelle composition may comprise at least 30% alpha casein protein. In certain cases, the protein content of a micelle composition may comprise at least 50% alpha casein protein. In various cases, the protein content of a micelle composition may comprise at least 90% or at least 95% alpha casein protein. The protein content of a micelle composition may comprise from 30% to 35%, 30% to 40%, 30% to 50%, 30% to 55%, 30% to 70%, 30% to 75%, 30% to 80%, 30% to 85%, 30% to 90%, 35% to 40%, 35% to 50%, 35% to 55%, 35% to 70%, 35% to 75%, 35% to 80%, 35% to 85%, 35% to 90%, 40% to 50%, 40% to 55%, 40% to 70%, 40% to 75%, 40% to 80%, 40% to 85%, 40% to 90%, 50% to 55%, 50% to 70%, 50% to 75%, 50% to 80%, 50% to 85%, 50% to 90%, 55% to 70%, 55% to 75%, 55% to 80%, 55% to 85%, 55% to 90%, 70% to 75%, 70% to 80%, 70% to 85%, 70% to 90%, 75% to 80%, 75% to 85%, 75% to 90%, 80% to 85%, 80% to 90%, 85% to 90% or 90 to 95% alpha casein protein. The protein content of a micelle composition may comprise about 30%, about 35%, about 40%, about 50%, about 55%, about 70%, about 75%, about 80%, about 85%, about 90%, or about 95% alpha casein protein. The protein content of a micelle composition may comprise at least 30%, at least 35%, at least 40%, at least 45%, at least 50%, at least 55%, at least 60%, at least 65%, at least 70%, at least 75%, at least 80%, at least 85%, or at least 90% alpha casein protein. The protein content of a micelle composition may comprise at most 40%, at most 45%, at most 50%, at most 55%, at most 60%, at most 65%, at most 70%, at most 75%, at most 80%, at most 85%, at most 90%, or at most 95% alpha casein protein.

The alpha casein protein (comprising either or both alpha-S1 and/or alpha-S2 caseins) may be produced recombinantly. In some cases, a micelle composition may comprise only recombinantly produced alpha casein protein. In certain cases, a micelle composition may comprise substantially only recombinantly produced alpha casein protein. For instance, alpha casein proteins may be at least 90%, at least 92%, at least 95%, at least 97%, or at least 99% recombinant alpha casein. Alternatively, a micelle composition may comprise a mixture of recombinantly produced and animal-obtained alpha casein proteins.

Depending on the host organism used to express the alpha casein, the alpha casein proteins may have a phosphorylation pattern different from animal-obtained alpha casein proteins. In some cases, the alpha casein protein may comprise no PTMs. In certain cases, the alpha casein protein may comprise substantially reduced PTMs. As used herein, substantially reduced PTMs generally refers to at least a 50% reduction of one or more types of PTMs as compared to the amount of PTMs in an animal-obtained alpha casein protein. For instance, alpha casein proteins may be at least 50%, at least 55%, at least 60%, at least 65%, at least 70%, at least 75%, at least 80%, at least 85%, at least 90%, at least 92%, at least 95%, at least 97%, or at least 99% less post-translationally modified as compared to animal-obtained alpha casein. In various cases, the alpha casein protein may comprise PTMs comparable to animal-obtained alpha casein PTMs. In some cases, recombinant alpha casein protein substantially or completely lacks a PTM of a native alpha casein protein.

The PTMs in the alpha casein protein may be modified chemically or enzymatically. In some cases, the alpha casein protein may comprise substantially reduced or no PTMs without chemical or enzymatic treatment. A micelle composition may be generated using alpha casein protein with reduced or no PTMs, wherein the lack of PTMs is not due to chemical or enzymatic treatments of the protein, such as producing an alpha casein protein through recombinant production where the recombinant protein lacks PTMs.

The phosphorylation in the recombinant alpha casein protein may comprise substantially reduced or no phosphorylation. For instance, alpha casein proteins may be at least 50%, at least 55%, at least 60%, at least 65%, at least 70%, at least 75%, at least 80%, at least 85%, at least 90%, at least 92%, at least 95%, at least 97%, or at least 99% less phosphorylated as compared to animal-obtained alpha casein or lack one or more specific sites of phosphorylation as compared to native alpha casein protein. In some cases, the alpha casein proteins may include at least one phosphorylation on a position that is different from a native phosphorylation. A micelle composition may be generated using alpha casein protein with reduced or no phosphorylation, wherein the lack of phosphorylation is not due to chemical or enzymatic treatments, such as where recombinant production provides alpha casein protein with reduced or no phosphorylation.

### Beta Casein

In various cases, a micelle composition as provided herein may include beta casein or a derivative thereof. In some embodiments, a micelle composition may comprise a significantly lower amount of beta casein protein as compared to an animal-obtained micelle composition (or animal-obtained micelle composition). The micelle composition described herein may be generated to comprise less than 10% beta casein protein. The protein content of the micelle composition may comprise less than 10%, less than 8%, less than 5%, less than 3%, less than 2%, less than 1%, or less than 0.5% beta casein protein. In some embodiments, the micelle composition described herein may not include any beta casein protein (e.g., the micelle composition may lack beta casein protein).

### Gamma Casein

In various cases, a micelle composition as provided herein may include gamma casein. In some embodiments, a micelle composition may comprise a significantly lower amount of gamma casein protein as compared to an animal-obtained micelle composition (or animal-obtained micelle composition). The micelle composition described herein may be generated to comprise less than 10% gamma casein protein. The protein content of the micelle composition may comprise less than 10%, less than 8%, less than 5%, less than 3%, less than 2%, less than 1%, or less than 0.5% gamma casein protein. In some embodiments, the micelle composition described herein may not include any gamma casein protein (e.g., the micelle composition may lack gamma casein protein).

### Kappa Casein

In some embodiments, a micelle composition or a micelle-like composition as provided herein may comprise kappa casein protein. The protein content of the micelle composition or micelle-like composition may comprise from 5% to 100% kappa casein protein. The protein content of the micelle composition or micelle-like composition may comprise at least 1% kappa casein protein. The protein content of the micelle composition or micelle-like composition may comprise at most 100%, at most 50%, or at most 30% kappa casein protein. The micelle composition or micelle-like composition may comprise from 1% to 5%, 1% to 7%, 1% to 10%, 1% to 12%, 1% to 15%, 1% to 18%, 1% to 20%, 1% to 25%, 1% to 30%, 5% to 7%, 5% to 10%, 5% to 12%, 5% to 15%, 5% to 18%, 5% to 20%, 5% to 25%, 5% to 30%, 7% to 10%, 7% to 12%, 7% to 15%, 7% to 18%, 7% to 20%, 7% to 25%, 7% to 30%, 10% to 12%, 10% to 15%, 10% to 18%, 10% to 20%, 10% to 25%, 10% to 30%, 12% to 15%, 12% to 18%, 12% to 20%, 12% to 25%, 12% to 30%, 15% to 18%, 15% to 20%, 15% to 25%, 15% to 30%, 18% to 20%, 18% to 25%, 18% to 30%, 20% to 25%, 20% to 30%, 25% to 30%, 30% to 35%, 35% to 40%, 40 to 45%, or 45% to 50% kappa casein protein. The protein content of the micelle composition or micelle-like composition may comprise about 1%, about 5%, about 7%, about 10%, about 12%, about 15%, about 18%, about 20%, about 25%, about 30%, about 35%, about 40%, about 45%, about 50%, about 60%, about 70%, about 80%, about 90%, or about 100% kappa casein protein. The protein content of the micelle composition or micelle-like composition may comprise at least 1%, at least 5%, at least 7%, at least 10%, at least 12%, at least 15%, at least 18%, at least 20%, at least 25%, at least 30%, at least 35%, at least 40%, or at least 45% kappa casein protein. The protein content of the micelle composition or micelle-like composition may comprise at most 5%, at most 7%, at most 10%, at most 12%, at most 15%, at most 18%, at most 20%, at most 25%, at most 30%, at most 35%, at most 40%, at most 45%, or at most 50% kappa casein protein. In some instances, a micelle composition or micelle-like composition may be produced using only kappa casein.

The kappa casein protein may be produced recombinantly. In some cases, a micelle composition or micelle-like composition may comprise only recombinantly produced kappa casein protein. In certain cases, a micelle composition or micelle-like composition may comprise substantially only recombinantly produced kappa casein protein. In various cases, kappa casein proteins may be at least 90%, at least 92%, at least 95%, at least 97%, or at least 99% recombinant kappa casein proteins. Alternatively, a micelle composition or micelle-like composition may comprise a mixture of recombinantly produced and animal-obtained kappa casein proteins.

Depending on the host organism used to express the kappa casein, the kappa casein proteins may have a PTM, such as a glycosylation or a phosphorylation pattern different from animal-obtained kappa casein protein. In some cases, the kappa casein protein in the composition herein comprises no PTMs. In some cases, the kappa casein protein may comprise substantially reduced PTMs. As used herein, substantially reduced PTMs generally refers to at least a 50% reduction of one or more types of PTMs as compared to the amount of PTMs in an animal-obtained kappa casein protein. For instance, kappa casein proteins may be at least 50%, at least 55%, at least 60%, at least 65%, at least 70%, at least 75%, at least 80%, at least 85%, at least 90%, at least 92%, at least 95%, at least 97%, or at least 99% less post-translationally modified as compared to animal-obtained kappa casein. In certain cases, the kappa casein protein may comprise PTMs comparable to animal-obtained kappa casein PTMs. In some cases, the kappa casein protein may lack any glycosylation, or lack a particular type of glycosylation such as N-linked or O-linked glycosylation.

In some cases, the kappa casein protein may comprise substantially reduced or no PTMs. A micelle composition may be generated using kappa casein protein with reduced or no PTMs, wherein the lack of or reduction of PTMs is not due to chemical or enzymatic treatments, such as by producing recombinant kappa protein in a host where the kappa casein protein is not post-translationally modified or the level of PTMs is substantially reduced.

The glycosylation in the kappa casein protein may be modified chemically or enzymatically. In some cases, the kappa casein protein may comprise substantially reduced or no glycosylation without chemical or enzymatic treatment. For instance, kappa casein proteins may be at least 50%, at least 55%, at least 60%, at least 65%, at least 70%, at least 75%, at least 80%, at least 85%, at least 90%, at least 92%, at least 95%, at least 97%, or at least 99% less glycosylated as compared to animal-obtained kappa casein. A micelle composition may be generated using kappa casein protein with reduced or no glycosylation, wherein the lack of glycosylation is not due to chemical or enzymatic treatments post recombinant production. In alternative embodiments, the kappa casein proteins may have increased glycosylation as compared to animal-obtained kappa casein. In some embodiments, the kappa casein proteins may comprise a glycosylation pattern that is different (e.g., may have glycosylated amino acid residues that are different) from animal-obtained kappa casein. In some embodiments, the kappa casein proteins may comprise N-linked glycosylation.

The phosphorylation in the kappa casein protein may be modified chemically or enzymatically. In some cases, the kappa casein protein may comprise substantially reduced or no phosphorylation without chemical or enzymatic treatment. For instance, kappa casein proteins may be at least 50%, at least 55%, at least 60%, at least 65%, at least 70%, at least 75%, at least 80%, at least 85%, at least 90%, at least 92%, at least 95%, at least 97%, or at least 99% less phosphorylated as compared to animal-obtained kappa casein. Micelles may be generated using kappa casein protein with reduced or no phosphorylation, wherein the lack of phosphorylation is not due to chemical or enzymatic treatments, such as by producing recombinant kappa protein in a host where the kappa casein protein is not post-translationally modified or the level of PTMs is substantially reduced.

The casein protein content of a micelle composition may comprise from about 5% kappa and about 95% alpha casein proteins to about 50% kappa and about 50% alpha casein proteins. The casein protein content of a micelle composition may comprise about 6% kappa and about 94% alpha, about 5% kappa and about 95% alpha, about 7% kappa and about 93% alpha, about 10% kappa and about 90%, alpha, about 12% kappa and about 88% alpha, about 15% kappa and about 85% alpha, about 17% kappa and about 83% alpha, about 20% kappa and about 80% alpha, about 25% kappa and about 75% alpha, about 30% kappa and about 70% alpha, about 35% kappa and about 65% alpha, about 40% kappa and about 60% alpha, about 45% kappa and about 55% alpha, or about 50% kappa and about 50% alpha.

The ratio of alpha casein protein to kappa casein protein in a micelle composition may be from about 1:1 to about 15:1. The ratio of alpha casein protein to kappa casein protein in a micelle composition may be about 1:1 or from 2:1 to 4:1, 2:1 to 6:1, 2:1 to 8:1, 2:1 to 10:1, 2:1 to 12:1, 2:1 to 14:1, 2:1 to 15:1, 4:1 to 6:1, 4:1 to 8:1, 4:1 to 10:1, 4:1 to 12:1, 4:1 to 14:1, 4:1 to 15:1, 6:1 to 8:1, 6:1 to 10:1, 6:1 to 12:1, 6:1 to 14:1, 6:1 to 15:1, 8:1 to 10:1, 8:1 to 12:1, 8:1 to 14:1, 8:1 to 15:1, 10:1 to 12:1, 10:1 to 14:1, 10:1 to 15:1, 12:1 to 14:1, 12:1 to 15:1, or 14:1 to 15:1. The ratio of alpha casein protein to kappa casein protein in a micelle composition may be about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1.

In some embodiments, a micelle composition comprises alpha and kappa casein proteins and does not include beta casein, and additionally, the alpha casein, kappa casein, or both the alpha and kappa casein lack PTM(s). For example, a micelle composition may comprise alpha casein lacking or substantially reduced in phosphorylation (as compared to alpha casein from animal-obtained milk) and kappa casein, or may comprise alpha casein lacking or substantially reduced in phosphorylation (as compared to alpha casein from animal-obtained milk) and kappa casein that lacks or is substantially reduced in glycosylation or phosphorylation or both glycosylation and phosphorylation (as compared to kappa casein from animal-obtained milk). In certain cases, a micelle composition may comprise alpha casein and comprise kappa casein where the kappa casein is lacking or substantially reduced in glycosylation or phosphorylation or both glycosylation and phosphorylation (as compared to kappa casein from animal-obtained milk). In various cases, a micelle composition comprises alpha casein, kappa casein, or both produced recombinantly in a bacterial host cell and that lack or are substantially reduced in one or more PTMs.

In some embodiments, a micelle composition as provided herein (and products made therefrom) may not include any dairy proteins other than alpha and kappa casein proteins. In some cases, a micelle composition as provided herein (and products made therefrom) may not include any whey proteins. In certain embodiments, a micelle composition as provided herein (and products made therefrom) may not include any animal-obtained dairy proteins.

In some embodiments, a micelle-like composition comprises kappa casein protein with reduced PTM(s) or that lacks PTM(s). In various embodiments, a micelle-like composition comprises kappa casein produced recombinantly in a bacterial host cell and that lacks or is substantially reduced in one or more PTMs. In various embodiments, a micelle-like composition comprises kappa casein produced recombinantly in a bacterial host cell and that lacks or is substantially reduced in glycosylation.

In some embodiments, a micelle-like composition as provided herein (and products made therefrom) may not include any dairy proteins other than kappa casein proteins. In some cases, a micelle-like composition as provided herein (and products made therefrom) may not include any whey proteins. In certain embodiments, a micelle-like composition as provided herein (and products made therefrom) may not include any animal-obtained dairy proteins.

### Micelles and Micelle-Like Compositions and Compositions Produced Therefrom

Micelle, such as micelles and sub-micelles in a micelle composition, may be from about 10 nm to about 900 nm. Micelle diameters may be at least 10 nm. Micelle diameters may be at most 900 nm. Micelle diameters may be from 10 nm to 20 nm, 10 nm to 50 nm, 10 nm to 100 nm, 10 nm to 150 nm, 10 nm to 200 nm, 10 nm to 250 nm, 10 nm to 300 nm, 10 nm to 350 nm, 10 nm to 400 nm, 10 nm to 450 nm, 10 nm to 500 nm, 10 nm to 600 nm, 10 nm to 700 nm, 10 nm to 800 nm, 20 nm to 50 nm, 20 nm to 100 nm, 20 nm to 150 nm, 20 nm to 200 nm, 20 nm to 250 nm, 20 nm to 300 nm, 20 nm to 350 nm, 20 nm to 400 nm, 20 nm to 450 nm, 20 nm to 500 nm, 20 nm to 600 nm, 20 nm to 700 nm, 20 nm to 800 nm, 50 nm to 100 nm, 50 nm to 150 nm, 50 nm to 200 nm, 50 nm to 250 nm, 50 nm to 300 nm, 50 nm to 350 nm, 50 nm to 400 nm, 50 nm to 450 nm, 50 nm to 500 nm, 50 nm to 600 nm, 50 nm to 700 nm, 50 nm to 800 nm, 100 nm to 150 nm, 100 nm to 200 nm, 100 nm to 250 nm, 100 nm to 300 nm, 100 nm to 350 nm, 100 nm to 400 nm, 100 nm to 450 nm, 100 nm to 500 nm, 100 nm to 600 nm, 100 nm to 700 nm, 100 nm to 800 nm, 150 nm to 200 nm, 150 nm to 250 nm, 150 nm to 300 nm, 150 nm to 350 nm, 150 nm to 400 nm, 150 nm to 450 nm, 150 nm to 500 nm, 150 nm to 900 nm, 200 nm to 250 nm, 200 nm to 300 nm, 200 nm to 350 nm, 200 nm to 400 nm, 200 nm to 450 nm, 200 nm to 500 nm, 200 nm to 900 nm, 250 nm to 300 nm, 250 nm to 350 nm, 250 nm to 400 nm, 250 nm to 450 nm, 250 nm to 500 nm, 250 nm to 900 nm, 300 nm to 350 nm, 300 nm to 400 nm, 300 nm to 450 nm, 300 nm to 500 nm, 300 nm to 900 nm, 350 nm to 400 nm, 350 nm to 450 nm, 350 nm to 500 nm, 400 nm to 450 nm, 400 nm to 500 nm, 400 nm to 900 nm, 450 nm to 500 nm, or 450 nm to 900 nm. Micelle diameters may be about 10 nm, about 20 nm, about 50 nm, about 100 nm, about 150 nm, about 200 nm, about 250 nm, about 300 nm, about 350 nm, about 400 nm, about 450 nm, about 500 nm, about 600 nm, about 700 nm, about 800 nm, or about 900 nm. Micelle diameters may be at least 10 nm, at least 20 nm, at least 50 nm, at least 100 nm, at least 150 nm, at least 200 nm, at least 250 nm, at least 300 nm, at least 350 nm, at least 400 nm, at least 450 nm, or at least 850 nm. Micelle diameters may be at most 20 nm, at most 50 nm, at most 100 nm, at most 150 nm, at most 200 nm, at most 250 nm, at most 300 nm, at most 350 nm, at most 400 nm, at most 450 nm, at most 500 nm, or at most 900 nm.

An average or mean size of micelle diameters in a micelle composition may be from about 50 nm to about 500 nm. Average or mean size of micelle diameters may be at least 50 nm. Average or mean size of micelle diameters may be at most 500 nm. Average or mean size of micelle diameters may be from 50 nm to 150 nm, 50 nm to 200 nm, 50 nm to 250 nm, 50 nm to 300 nm, 50 nm to 350 nm, 50 nm to 400 nm, 50 nm to 450 nm, 50 nm to 500 nm, 100 nm to 150 nm, 100 nm to 200 nm, 100 nm to 250 nm, 100 nm to 300 nm, 100 nm to 350 nm, 100 nm to 400 nm, 100 nm to 450 nm, 100 nm to 500 nm, 150 nm to 200 nm, 150 nm to 250 nm, 150 nm to 300 nm, 150 nm to 350 nm, 150 nm to 400 nm, 150 nm to 450 nm, 150 nm to 500 nm, 200 nm to 250 nm, 200 nm to 300 nm, 200 nm to 350 nm, 200 nm to 400 nm, 200 nm to 450 nm, 200 nm to 500 nm, 250 nm to 300 nm, 250 nm to 350 nm, 250 nm to 400 nm, 250 nm to 450 nm, 250 nm to 500 nm, 300 nm to 350 nm, 300 nm to 400 nm, 300 nm to 450 nm, 300 nm to 500 nm, 350 nm to 400 nm, 350 nm to 450 nm, 350 nm to 500 nm, 400 nm to 450 nm, 400 nm to 500 nm, or 450 nm to 500 nm. Average or mean size of micelle diameters may be about 50 nm, about 100 nm, about 150 nm, about 200 nm, about 250 nm, about 300 nm, about 350 nm, about 400 nm, about 450 nm, or about 500 nm. Average or mean size of micelle diameters may be at least 50 nm, at least 100 nm, at least 150 nm, at least 200 nm, at least 250 nm, at least 300 nm, at least 350 nm, at least 400 nm, or at least 450 nm. Average or mean size of micelle diameters may be at most 75 nm, at most 100 nm, at most 150 nm, at most 200 nm, at most 250 nm, at most 300 nm, at most 350 nm, at most 400 nm, at most 450 nm, or at most 500 nm.

Micelle-like structure diameters, such as micelle-like and sub-micelle-like structures in a micelle-like composition, may be from about 10 nm to about 900 nm. Micelle-like structure diameters may be at least 10 nm. Micelle-like structure diameters may be at most 900 nm. Micelle-like structure diameters may be from 10 nm to 20 nm, 10 nm to 50 nm, 10 nm to 100 nm, 10 nm to 150 nm, 10 nm to 200 nm, 10 nm to 250 nm, 10 nm to 300 nm, 10 nm to 350 nm, 10 nm to 400 nm, 10 nm to 450 nm, 10 nm to 500 nm, 10 nm to 600 nm, 10 nm to 700 nm, 10 nm to 800 nm, 20 nm to 50 nm, 20 nm to 100 nm, 20 nm to 150 nm, 20 nm to 200 nm, 20 nm to 250 nm, 20 nm to 300 nm, 20 nm to 350 nm, 20 nm to 400 nm, 20 nm to 450 nm, 20 nm to 500 nm, 20 nm to 600 nm, 20 nm to 700 nm, 20 nm to 800 nm, 50 nm to 100 nm, 50 nm to 150 nm, 50 nm to 200 nm, 50 nm to 250 nm, 50 nm to 300 nm, 50 nm to 350 nm, 50 nm to 400 nm, 50 nm to 450 nm, 50 nm to 500 nm, 50 nm to 600 nm, 50 nm to 700 nm, 50 nm to 800 nm, 100 nm to 150 nm, 100 nm to 200 nm, 100 nm to 250 nm, 100 nm to 300 nm, 100 nm to 350 nm, 100 nm to 400 nm, 100 nm to 450 nm, 100 nm to 500 nm, 100 nm to 600 nm, 100 nm to 700 nm, 100 nm to 800 nm, 150 nm to 200 nm, 150 nm to 250 nm, 150 nm to 300 nm, 150 nm to 350 nm, 150 nm to 400 nm, 150 nm to 450 nm, 150 nm to 500 nm, 150 nm to 900 nm, 200 nm to 250 nm, 200 nm to 300 nm, 200 nm to 350 nm, 200 nm to 400 nm, 200 nm to 450 nm, 200 nm to 500 nm, 200 nm to 900 nm, 250 nm to 300 nm, 250 nm to 350 nm, 250 nm to 400 nm, 250 nm to 450 nm, 250 nm to 500 nm, 250 nm to 900 nm, 300 nm to 350 nm, 300 nm to 400 nm, 300 nm to 450 nm, 300 nm to 500 nm, 300 nm to 900 nm, 350 nm to 400 nm, 350 nm to 450 nm, 350 nm to 500 nm, 400 nm to 450 nm, 400 nm to 500 nm, 400 nm to 900 nm, 450 nm to 500 nm, or 450 nm to 900 nm. Micelle-like structure diameters may be about 10 nm, about 20 nm, about 50 nm, about 100 nm, about 150 nm, about 200 nm, about 250 nm, about 300 nm, about 350 nm, about 400 nm, about 450 nm, about 500 nm, about 600 nm, about 700 nm, about 800 nm, or about 900 nm. Micelle-like structure diameters may be at least 10 nm, at least 20 nm, at least 50 nm, at least 100 nm, at least 150 nm, at least 200 nm, at least 250 nm, at least 300 nm, at least 350 nm, at least 400 nm, at least 450 nm, or at least 850 nm. Micelle-like structure diameters may be at most 20 nm, at most 50 nm, at most 100 nm, at most 150 nm, at most 200 nm, at most 250 nm, at most 300 nm, at most 350 nm, at most 400 nm, at most 450 nm, at most 500 nm, or at most 900 nm.

An average or mean size of micelle-like or sub-micelle-like structure diameters in a micelle-like composition may be from about 20 nm to about 700 nm, such as from about 50 nm to about 500 nm. Average or mean size of micelle-like structure diameters may be at least about 20 nm, such as at least about 50 nm. Average or mean size of micelle-like structure diameters may be at most about 700 nm, such as at most about 500 nm. Average or mean size of micelle-like structure diameters may be from about 20 nm to about 50 nm, about 20 nm to about 100 nm, about 20 nm to about 150 nm, about 20 nm to about 200 nm, about 20 nm to about 250 nm, about 20 nm to about 300 nm, about 20 nm to about 350 nm, about 20 nm to about 400 nm, about 20 nm to about 450 nm, about 20 nm to about 500 nm, about 20 nm to about 500 nm, about 20 nm to about 550 nm, about 20 nm to about 600 nm, about 20 nm to about 650 nm, about 20 nm to about 700 nm, about 50 nm to about 100 nm, about 50 nm to about 150 nm, about 50 nm to about 200 nm, about 50 nm to about 250 nm, about 50 nm to about 300 nm, about 50 nm to about 350 nm, about 50 nm to about 400 nm, about 50 nm to about 450 nm, about 50 nm to about 500 nm, about 50 nm to about 550 nm, about 50 nm to about 600 nm, about 50 nm to about 650 nm, about 50 nm to about 700 nm, about 100 nm to about 150 nm, about 100 nm to about 200 nm, about 100 nm to about 250 nm, about 100 nm to about 300 nm, about 100 nm to about 350 nm, about 100 nm to about 400 nm, about 100 nm to about 450 nm, about 100 nm to about 500 nm, about 100 nm to about 550 nm, about 100 nm to about 600 nm, about 100 nm to about 650 nm, about 100 nm to about 700 nm, about 150 nm to about 200 nm, about 150 nm to about 250 nm, about 150 nm to about 300 nm, about 150 nm to about 350 nm, about 150 nm to about 400 nm, about 150 nm to about 450 nm, about 150 nm to about 500 nm, about 150 nm to about 550 nm, about 150 nm to about 600 nm, about 150 nm to about 700 nm, about 200 nm to about 250 nm, about 200 nm to about 300 nm, about 200 nm to about 350 nm, about 200 nm to about 400 nm, about 200 nm to about 450 nm, about 200 nm to about 500 nm, about 200 nm to about 550 nm, about 200 nm to about 600 nm, about 200 nm to about 650 nm, about 200 nm to about 700 nm, about 250 nm to about 300 nm, about 250 nm to about 350 nm, about 250 nm to about 400 nm, about 250 nm to about 450 nm, about 250 nm to about 500 nm, about 300 nm to about 350 nm, about 300 nm to about 400 nm, about 300 nm to about 450 nm, about 300 nm to about 500 nm, about 300 nm to about 550 nm, about 300 nm to about 600 nm, about 300 nm to about 650 nm, about 300 nm to about 700 nm, about 350 nm to about 400 nm, about 350 nm to about 450 nm, about 350 nm to about 500 nm, about 350 nm to about 550 nm, about 350 nm to about 600 nm, about 350 nm to about 650 nm, about 350 nm to about 700 nm, about 400 nm to about 450 nm, about 400 nm to about 500 nm, about 400 nm to about 550 nm, about 400 nm to about 600 nm, about 400 nm to about 650 nm, about 400 nm to about 700 nm, about 450 nm to about 500 nm, about 450 nm to about 550 nm, about 450 nm to about 600 nm, about 450 nm to about 650 nm, about 450 nm to about 700 nm, about 500 nm to about 550 nm, about 500 nm to about 600 nm, about 500 nm to about 650 nm, about 500 nm to about 700 nm, about 550 nm to about 600 nm, about 550 nm to about 650 nm, about 550 nm to about 700 nm, about 600 nm to about 650 nm, about 600 nm to about 700 nm, or about 650 nm to about 700 nm. In a particular embodiment, the average or mean size of micelle-like structure diameter in a micelle-like composition may be from about 100 nm to about 250 nm. In another particular embodiment, the average or mean size of micelle-like structure diameter in a micelle-like composition may be from about 600 nm to about 700 nm. In another particular embodiment, the average or mean size of sub-micelle-like structure diameter in a micelle-like composition may be from about 20 nm to about 50 nm. Average or mean size of micelle-like structure diameters may be about 20 nm, 50 nm, about 100 nm, about 150 nm, about 200 nm, about 250 nm, about 300 nm, about 350 nm, about 400 nm, about 450 nm, about 500 nm, about 550 nm, about 600 nm, about 650 nm, or about 700 nm. Average or mean size of micelle-like structure diameters may be at least about 20 nm, at least about 50 nm, at least about 100 nm, at least about 150 nm, at least about 200 nm, at least about 250 nm, at least about 300 nm, at least about 350 nm, at least about 400 nm, at least about 450 nm, at least about 500 nm, at least about 550 nm, at least about 600 nm, or at least about 650 nm. Average or mean size of micelle-like structure diameters may be at most about 50 nm, at most about 75 nm, at most about 100 nm, at most about 150 nm, at most about 200 nm, at most about 250 nm, at most about 300 nm, at most about 350 nm, at most about 400 nm, at most about 450 nm, at most about 500 nm, at most about 550 nm, at most about 600 nm, at most about 650 nm, or at most about 700 nm.

Salts: The micelle compositions and micelle-like compositions may comprise alpha, beta, kappa, and/or gamma casein proteins as described elsewhere herein. In some embodiments, a micelle composition includes alpha casein and kappa casein, but does not include beta casein. In certain embodiments, a micelle-like composition includes kappa casein, but does not include alpha casein and beta casein. Other combinations of casein proteins are also within the scope of this disclosure. Micelle formation may comprise addition of one or more of various salts to a solution comprising a casein mixture. Salts that may be added to a casein mixture may include calcium, phosphorous, citrate, potassium, sodium, zinc, manganese, and/or chloride salts. In some cases, salt is comprised within the micelles. In various cases, a portion of the salt is comprised in the micelles and another portion of the salt is in a solution that contains the micelles (e.g., “outside” the micelles), such as when micelles are in a liquid colloid.

A liquid colloid containing casein micelles or micelle-like structures may comprise a calcium salt or another divalent cation salt. The calcium salt, or other divalent cation salt, may be selected from calcium chloride, calcium carbonate, calcium citrate, calcium glubionate, calcium lactate, calcium gluconate, calcium acetate, equivalents thereof, and/or combinations thereof. The concentration of a calcium salt in a liquid colloid may be from about 10 mM to about 55 mM. The concentration of a calcium salt in a liquid colloid may be at least 10 mM. The concentration of a calcium salt in a liquid colloid may be at most 50 mM. In some embodiments, the concentration of a calcium salt in a liquid colloid may be 28 mM or no more than 28 mM or may be 55 mM or no more than 55 mM. The concentration of a calcium salt in a liquid colloid may be from 10 mM to 15 mM, 10 mM to 20 mM, 10 mM to 25 mM, 10 mM to 30 mM, 10 mM to 35 mM, 10 mM to 40 mM, 10 mM to 45 mM, 10 mM to 50 mM, 10 mM to 55 mM, 15 mM to 20 mM, 15 mM to 25 mM, 15 mM to 30 mM, 15 mM to 35 mM, 15 mM to 40 mM, 15 mM to 45 mM, 15 mM to 50 mM, 15 mM to 55 mM, 20 mM to 25 mM, 20 mM to 30 mM, 20 mM to 35 mM, 20 mM to 40 mM, 20 mM to 45 mM, 20 mM to 50 mM, 20 mM to 55 mM, 25 mM to 30 mM, 25 mM to 35 mM, 25 mM to 40 mM, 25 mM to 45 mM, 25 mM to 50 mM, 25 mM to 55 mM, 30 mM to 35 mM, 30 mM to 40 mM, 30 mM to 45 mM, 30 mM to 50 mM, 30 mM to 55 mM, 35 mM to 40 mM, 35 mM to 45 mM, 35 mM to 50 mM, 35 mM to 55 mM, 40 mM to 45 mM, 40 mM to 50 mM, 40 mM to 55 mM, 45 mM to 50 mM, 45 mM to 55 mM, or 50 mM to 55 mM.

The concentration of a calcium salt in a liquid colloid may be about 10 mM, about 15 mM, about 20 mM, about 25 mM, about 30 mM, about 35 mM, about 40 mM, about 45 mM, about 50 mM, or about 55 mM. The concentration of a calcium salt in a liquid colloid may be at least 10 mM, at least 15 mM, at least 20 mM, at least 25 mM, at least 30 mM, at least 35 mM, at least 40 mM, at least 45 mM, or at least 50 mM. The concentration of a calcium salt in a liquid colloid may be at most 15 mM, at most 20 mM, at most 25 mM, at most 30 mM, at most 35 mM, at most 40 mM, at most 45 mM, at most 50 mM, or at most 55 mM.

A liquid colloid containing casein micelles or micelle-like structures may comprise a phosphate salt. The phosphate salt may be selected from orthophosphates such as monosodium (dihydrogen) phosphate, disodium phosphate, trisodium phosphate, monopotassium (dihydrogen) phosphate, dipotassium phosphate, tripotassium phosphate; pyrophosphates such as disodium or dipotassium pyrophosphate, trisodium or tripotassium pyrophosphate, tetrasodium or tetrapotassium pyrophosphate; polyphosphates such as pentasodium or potassium tripolyphosphate, sodium or potassium tetrapolyphosphate, sodium or potassium hexametaphosphate. The concentration of a phosphate salt in a liquid colloid may be from about 8 mM to about 45 mM. The concentration of a phosphate salt in a liquid colloid may be at least 8 mM. The concentration of a phosphate salt in a liquid colloid may be at most 25 mM, at most 30 mM, at most 40 mM, or at most 45 mM. In embodiments, a liquid colloid containing casein micelles or micelle-like structures does not comprise a phosphate salt.

The concentration of a phosphate salt in a liquid colloid may be from 8 mM to 10 mM, 8 mM to 15 mM, 8 mM to 20 mM, 8 mM to 25 mM, 8 mM to 30 mM, 8 mM to 35 mM, 8 mM to 40 mM, 8 mM to 45 mM, 10 mM to 15 mM, 10 mM to 20 mM, 10 mM to 25 mM, 10 mM to 30 mM, 10 mM to 35 mM, 10 mM to 40 mM, 10 mM to 45 mM, 15 mM to 20 mM, 15 mM to 25 mM, 15 mM to 30 mM, 15 mM to 35 mM, 15 mM to 40 mM, 15 mM to 45 mM, 20 mM to 25 mM, 20 mM to 30 mM, 20 mM to 35 mM, 20 mM to 40 mM, 20 mM to 45 mM, 25 mM to 30 mM, 25 mM to 35 mM, 25 mM to 40 mM, 25 mM to 45 mM, 30 mM to 35 mM, 30 mM to 40 mM, 30 mM to 45 mM, 35 mM to 40 mM, 35 mM to 45 mM, or 40 mM to 45 mM. The concentration of a phosphate salt in a liquid colloid may be about 8 mM, about 10 mM, about 15 mM, about 20 mM, about 25 mM, about 30 mM, about 35 mM, about 40 mM, or about 45 mM. The concentration of a phosphate salt in a liquid colloid may be at least 8 mM, at least 10 mM, at least 15 mM, at least 20 mM, at least 25 mM, at least 30 mM, at least 35 mM, or at least 40 mM. The concentration of a phosphate salt in a liquid colloid may be at most 10 mM, at most 15 mM, at most 20 mM, at most 25 mM, at most 30 mM, at most 35 mM, at most 40 mM, or at most 45 mM.

A liquid colloid containing casein micelles or micelle-like structures may comprise a citrate salt. The citrate salt may be selected from calcium citrate, potassium citrate, sodium citrate, trisodium citrate, tripotassium citrate, or equivalents thereof. The concentration of a citrate salt in a liquid colloid may be from about 2 mM to about 20 mM. The concentration of a citrate salt in a liquid colloid may be at least 2 mM. The concentration of a citrate salt in a liquid colloid may be at most 15 mM or at most 20 mM. The concentration of a citrate salt in a liquid colloid may be from 2 mM to 4 mM, 2 mM to 6 mM, 2 mM to 8 mM, 2 mM to 10 mM, 2 mM to 12 mM, 2 mM to 14 mM, 2 mM to 16 mM, 2 mM to 18 mM, 2 mM to 20 mM, 4 mM to 6 mM, 4 mM to 8 mM, 4 mM to 10 mM, 4 mM to 12 mM, 4 mM to 14 mM, 4 mM to 16 mM, 4 mM to 18 mM, 4 mM to 20 mM, 6 mM to 8 mM, 6 mM to 10 mM, 6 mM to 12 mM, 6 mM to 14 mM, 6 mM to 16 mM, 6 mM to 18 mM, 6 mM to 20 mM, 8 mM to 10 mM, 8 mM to 12 mM, 8 mM to 14 mM, 8 mM to 16 mM, 8 mM to 18 mM, 8 mM to 20 mM, 10 mM to 12 mM, 10 mM to 14 mM, 10 mM to 16 mM, 10 mM to 18 mM, 10 mM to 20 mM, 12 mM to 14 mM, 12 mM to 16 mM, 12 mM to 18 mM, 12 mM to 20 mM, 14 mM to 16 mM, 14 mM to 18 mM, 14 mM to 20 mM, 16 mM to 18 mM, 16 mM to 20 mM, or 18 mM to 20 mM. The concentration of a citrate salt in a liquid colloid may be about 2 mM, about 4 mM, about 6 mM, about 8 mM, about 10 mM, about 12 mM, about 14 mM, about 16 mM, about 18 mM, or about 20 mM. The concentration of a citrate salt in a liquid colloid may be at least 2 mM, at least 4 mM, at least 6 mM, at least 8 mM, at least 10 mM, at least 12 mM, at least 14 mM, at least 16 mM, or at least 18 mM. The concentration of a citrate salt in a liquid colloid may be at most 4 mM, at most 6 mM, at most 8 mM, at most 10 mM, at most 12 mM, at most 14 mM, at most 16 mM, at most 18 mM, or at most 20 mM. In some embodiments, a liquid colloid containing casein micelles or micelle-like structures does not comprise a citrate salt.

A liquid colloid containing casein micelles or micelle-like structures may comprise a combination of salts. In some embodiments, the liquid colloid may comprise calcium, phosphate, and citrate salts. In some cases, a ratio of calcium, phosphate, and citrate salts in a liquid colloid may be from about 3:2:1 to about 6:4:1. A ratio of calcium, phosphate, and citrate salts in a liquid colloid may be about 3:1:1, about 3:2:1, about 3:3:1, about 4:2:1, about 4:3:1, about 4:4:1, about 5:2:1, about 5:2:2, about 5:3:1, about 5:4:1, about 5:5:1, about 5:3:2, about 5:4:2, about 6:1:1, about 6:2:1, about 6:3:1, or about 6:4:1. In some embodiments, a ratio of divalent cation salts to total phosphate and/or citrate salts may be from about 1:1 to about 3:1.

Micelle formation in a liquid colloid may require solubilization of casein proteins in a solvent such as water. Salts may be added after the solubilization of casein proteins in a solvent. Alternatively, salts and casein proteins may be added to the solution simultaneously. Salts may be added more than once during micelle formation. For instance, calcium salts, phosphate salts, and citrate salts may be added at regular intervals or in a continuous titration process and mixed in a solution comprising casein proteins until a micellar liquid colloid of desired quality is generated. For example, salts may be added at a regular interval until the colloid reaches a desired turbidity. Different salts may be added at different times during the micelle formation process. For instance, calcium salts may be added before the addition of phosphate and citrate salts, citrate salts may be added before the addition of calcium and phosphate salts, or phosphate salts might be added before the addition of calcium and citrate salts.

In some embodiments, micelle compositions and micelle-like compositions as described herein may include adding or providing a crosslinking agent. Crosslinking agents may include transglutaminases, tyrosinases, laccases, peroxidases, or glucose oxidases. Additionally, the crosslinking agent may be added or provided under conditions for inducing intra-micellar crosslinking. Accordingly, the methods may form or produce micelles or micelle-like structures comprising caseins (e.g., alpha casein and kappa casein, or kappa casein alone). In some embodiments, the crosslinking agents are used in preparations suitable for applications in the food industry. For example, the crosslinking agents may be tested for toxicity and/or immunogenicity. In some embodiments, the crosslinking agent is a Generally Recognized as Safe (GRAS) certified cross-linking agent (e.g., a GRAS-certified transglutaminase). In various embodiments, the cross-linking agent (e.g., the transglutaminase) is formulated without lactose or any other dairy product in its production.

In various embodiments, micelle compositions and micelle-like compositions may include adding or providing an alpha casein, wherein the alpha casein has reduced or lacks PTM features. For example, the alpha casein may have reduced phosphorylation or may lack phosphorylation. The methods may further include adding or providing a crosslinking agent under conditions for crosslinking the alpha casein. In some cases, the methods may further include mixing kappa casein with the crosslinked alpha casein under conditions configured to induce micelle formation. As such, the methods may form or produce micelles comprising the alpha casein and the kappa casein in a form suitable for a dairy-like ingredient.

One or more caseins (e.g., the alpha casein and kappa casein) may be incubated together at the same time, or substantially the same time, as adding the crosslinking agent. One or more caseins (e.g., the alpha casein and kappa casein) may be incubated together prior to adding the crosslinking agent. In certain cases, the crosslinking agent may be added from about 30 minutes to about 24 hours after the caseins (e.g., the alpha casein and kappa casein) are incubated together. The crosslinking agent may be added from about 1 hour to about 12 hours after the caseins (e.g., the alpha casein and kappa casein) are incubated together. The crosslinking agent may be added from 30 minutes to 24 hours, 1 hour to 20 hours, 2 hours to 18 hours, 5 hours to 15 hours, or 8 hours to 12 hours after the caseins are incubated together. The crosslinking agent may be added about 30 minutes, about 1 hour, about 2 hours, about 5 hours, about 8 hours, about 12 hours, about 15 hours, about 18 hours, about 20 hours, or about 24 hours after the caseins are incubated together. The crosslinking agent may be added at least 30 minutes, at least 1 hour, at least 2 hours, at least 5 hours, at least 8 hours, at least 12 hours, at least 15 hours, at least 18 hours, at least 20 hours, or at least 24 hours after the caseins are incubated together. The crosslinking agent may be added at most 1 hour, at most 2 hours, at most 5 hours, at most 8 hours, at most 12 hours, at most 15 hours, at most 18 hours, at most 20 hours, at most 24 hours, or at most 36 hours after the caseins are incubated together.

A micelle composition or a micelle-like composition may be generated by incubating the crosslinking agent with casein proteins (e.g., at 40° C. for 1 hour). The incubation step may occur at a temperature from 10° C. to 60° C., such as at a temperature of about 10° C., 15° C., 20° C., 25° C., 30° C., 32° C., 35° C., 37° C., 40° C., 42° C., 45° C., 47° C., 49° C., 50° C., or 60° C. or any range between these temperatures. The incubation step may occur for a time period from 1 minute to 24 hours. The incubation step may occur for a time period from 30 minutes to 22 hours, 1 hour to 20 hours, 2 hours to 18 hours, 5 hours to 15 hours, or 8 hours to 12 hours. The incubation step may occur for a time period of at least 1 minute, at least 30 minutes, at least 1 hour, at least 2 hours, at least 5 hours, at least 8 hours, at least 12 hours, at least 14 hours, at least 16 hours, at least 18 hours, at least 20 hours, or at least 22 hours. The incubation step may occur for a time period of at most 5 minutes, at most 30 minutes, at most 1 hour, at most 2 hours, at most 5 hours, at most 8 hours, at most 12 hours, at most 14 hours, at most 16 hours, at most 18 hours, at most 20 hours, or at most 24 hours.

In various embodiments, the crosslinking agent may be inactivated with an inactivation step (e.g., by exposing the crosslinking agent to high temperature). The inactivation step may occur at a temperature from 60° C. to 90° C., such as at a temperature of about 60° C., about 65° C., about 70° C., about 75° C., about 80° C., about 85° C., about 90° C., or any range between these temperatures. The inactivation step may occur from a time period of 1 minute to 1 hour. The inactivation step may occur for a time period from 10 minutes to 1 hour. The inactivation step may occur for a time period of at least 1 minute, at least 5 minutes, at least 10 minutes, at least 15 minutes, at least 20 minutes, at least 25 minutes, at least 30 minutes, at least 45 minutes, or at least 1 hour. In some embodiments, the crosslinking agent is not inactivated, or is not inactivated by a high temperature step.

In various embodiments, the crosslinking agent may be added prior to the step of inducing micelle formation. In some embodiments, the crosslinking agent may be added subsequent to the step of inducing micelle formation. For example, the crosslinking agent may be added to casein proteins after the micelles have been induced and formed. In certain embodiments, the crosslinking agent may be added during (e.g., in parallel with) the step of inducing micelle formation.

### II. Dairy-Like Products & Methods of Making Dairy-Like Products

Dairy-like products can be formed using micelle compositions or micelle-like compositions as provided herein. The dairy-like product may be an edible composition (e.g., edible by a human subject). In some embodiments, the dairy-like product may be a milk-like product, a yogurt-like product, a curd-like product, a cheese-like product, a cream-like product, an ice cream-like product, or any combination thereof. The dairy-like product may further include one or more of a fat, a sugar, a flavoring, or a colorant.

The dairy-like product may include a dairy-like ingredient. In certain cases, one or more dairy-like ingredients may be combined with each other to form a dairy-like product. In various cases, one or more dairy-like ingredients may be combined with one or more other ingredients (e.g., non-dairy ingredients) to form a dairy-like product. In some cases, the one or more other ingredients may include sugars, fats, stabilizers, flavoring agents, and colorants.

In certain instances, the dairy-like product or dairy-like ingredient may be in a powder form. The powder form of the dairy-like product or ingredient may be formed or produced by spray drying, roller drying, fluid bed drying, drum drying, freeze drying, drying with ethanol, and/or evaporating a dairy-like product or ingredient as provided herein.

A powder may include a micelle composition or a micelle-like composition as provided herein. In some cases, the casein content of the powder may be from about 50% to about 90%. The casein content of the powder may be from 45% to 95%, 50% to 90%, 55% to 85%, 60% to 80%, 65% to 75%. The casein content of the powder may be about 45%, about 40%, about 55%, about 60%, about 65%, about 70%, about 75%, about 80%, about 85%, about 90%, or about 95%. The casein content of the powder may be at least 40%, at least 50%, at least 60%, at least 70%, at least 80%, or at least 90%. The casein content of the powder may be at most 45%, at most 55%, at most 65%, at most 75%, at most 85%, or at most 95%.

Micelles or micelle-like structures as provided herein may be dried to produce a micellar or micellar-like casein containing protein powder. Methods for drying micelles, micelle compositions, micelle-like structures, and/or micelle-like compositions may include, but are not limited to, spray drying, roller drying, fluid bed drying, drum drying, freeze drying, drying with ethanol, and evaporating.

Casein containing protein powder may be generated by subjecting the micelles within a micelle composition or the micelle-like structures within a micelle-like composition to salt precipitation. Casein containing protein powder may be generated by subjecting the micelles within a micelle composition or the micelle-like structures within a micelle-like composition to acid precipitation. Herein described casein containing protein powder may be used as an ingredient in a consumable food product. For instance, the casein containing protein powder may be used as an ingredient in the production of milk, milkshakes, beverages, snacks, creamers, condensed milk, cream, ice-cream, yogurt, mozzarella cheese analogue, curd, cheese and/or any other dairy products.

As described herein, methods of making a dairy-like product or dairy-like ingredient may include adding or providing caseins. In some cases, the casein may include an alpha casein and/or a kappa casein. The casein may include one or more non-native PTM features. For example, the alpha casein, the kappa casein, or both the alpha casein and the kappa casein may include a non-native PTM feature. The casein may include a reduced number of one or more PTM features. The casein may lack PTM features. For example, the alpha casein may be reduced in or lack phosphorylation or may include non-native phosphorylation sites and/or the kappa casein may be reduced in or lack glycosylation or may include non-native glycosylation sites. Methods of making the dairy-like ingredient may further include inducing micelle formation as discussed herein (e.g., by salt addition and by addition of a crosslinking agent) or using micelle-like compositions.

Methods of making a dairy-like product or dairy-like ingredient may include adding additional ingredients to the micelle compositions or micelle-like compositions described herein.

Additional components may be added to a liquid colloid containing micelles and/or micelle-like structures that the liquid colloid is then milk-like and used for curd and/or cheese or yogurt formation. In some embodiments, fat can be added to a liquid colloid. In some cases, fats may be essentially free of animal-obtained fats. Fats used herein may include plant-based fats such as canola oil, sunflower oil, coconut oil, palm oil, or combinations thereof. The concentration of fats may be about 0% to about 5% in the liquid colloid. The concentration of fats may be at least 0.5% or about 1%. The concentration of fats may be at most 5%. The concentration of fats may be about 0%, about 0.1%, about 0.5%, about 1%, about 2%, about 3%, about 4%, or about 5%. The concentration of fats may be from 0 to 0.5%, 0.5% to 1%, 1% to 3%, 1% to 4%, or 1% to 5%. The concentration of fats may be at most 2%, 3%, 4%, or 5%.

A liquid colloid as described herein may further comprise one or more sugars. Sugars used herein may include plant-based sugars (e.g., plant-based monosaccharides, disaccharides, and/or oligosaccharides). Examples of sugars include sucrose, glucose, fructose, galactose, lactose, maltose, mannose, allulose, tagatose, xylose, and arabinose.

In some case, fats may be emulsified into micelle compositions and micelle-like compositions that are in the form of a liquid colloid using a sonication, sheer mixing under temperature treatment, or high-pressure homogenization process. An emulsifier such as soy lecithin or xanthan gum may be used to secure a stable emulsion.

In certain instances, the dairy-like product may be a cheese-like product. A liquid colloid may be used to make a cheese-like product. Micelles or micelle-like structures, as provided herein, may be present in a liquid colloid, where a substantial portion of the micelles or micelle-like structures remain in suspension in the liquid. In some embodiments, the liquid colloid is treated to form a coagulated colloid. In certain embodiments, the treatment is a reduction of pH of the liquid colloid. The reduction of pH of the liquid colloid to generate coagulated colloid may be conducted by adding one or more acids or acidifying with one or more microorganisms.

The cheese-like product may be a soft cheese, a hard cheese, a pasta-filata cheese, an aged cheese, or a matured cheese. In some cases, the cheese-like product may be a paneer, a cream cheese, or a cottage cheese. In certain cases, the cheese-like product may be a cheddar cheese, a Swiss cheese, or a gouda cheese. In various cases, the cheese-like product may be a mozzarella.

The moisture retention of the cheese-like product may be from about 30% to about 80%. The moisture retention of the cheese-like product may be less than about 80%. The moisture retention of the cheese-like product may be at most 80%. The moisture retention of the cheese-like product may be from 30% to 80%, 40% to 65%, 45% to 60%, 50% to 55%, 30% to 65%, 35% to 60%, 40% to 55%, 45% to 50%, 40% to 70%, 45% to 65%, or 50% to 60%. The moisture retention of the cheese-like product may be about 30%, about 35%, about 40%, about 45%, about 55%, about 60%, about 65%, about 70%, about 75%, or about 80%. The moisture retention of the cheese-like product may be at least about 30%, at least about 35%, at least about 40%, at least about 45%, at least about 50%, at least about 55%, at least about 60%, at least about 65%, at least about 70%, or at least about 75%. The moisture retention of the cheese-like product may be at most about 35%, at most about 40%, at most about 45%, at most about 50%, at most about 55%, at most about 60%, at most about 65%, at most about 70%, at most about 75%, or at most about 80%.

Fats may be added, for example, for the generation of a coagulated colloid or curds such that in a final dairy product (e.g., a cheese-like product or yogurt-like product) the concentration of fat is from about 0% to about 50%. The fat may be from a plant (e.g., the fat may be a plant-based fat). Generally, the concentration of fat in the final dairy product is greater than 0%. For example, the concentration of fat in a dairy product made from a liquid colloid may be about 5%, 10%, 15%, 20%, 25%, 30%, 35%, 40%, 45%, or 50%. The concentration of fat in the dairy product made from the micelle compositions and micelle-like compositions herein may be from 1% to 50%. The concentration of fat in the dairy product made from the micelle compositions and micelle-like compositions herein may be at least 1%. The concentration of fat in the dairy product may be at most 50%. The concentration of fat in the dairy product made from the micelle compositions and micelle-like compositions herein may be from 1% to 5%, 1% to 10%, 1% to 15%, 1% to 20%, 1% to 25%, 1% to 30%, 1% to 35%, 1% to 40%, 1% to 45%, 1% to 50%, 5% to 10%, 5% to 15%, 5% to 20%, 5% to 25%, 5% to 30%, 5% to 35%, 5% to 40%, 5% to 45%, 5% to 50%, 10% to 15%, 10% to 20%, 10% to 25%, 10% to 30%, 10% to 35%, 10% to 40%, 10% to 45%, 10% to 50%, 15% to 20%, 15% to 25%, 15% to 30%, 15% to 35%, 15% to 40%, 15% to 45%, 15% to 50%, 20% to 25%, 20% to 30%, 20% to 35%, 20% to 40%, 20% to 45%, 20% to 50%, 25% to 30%, 25% to 35%, 25% to 40%, 25% to 45%, 25% to 50%, 30% to 35%, 30% to 40%, 30% to 45%, 30% to 50%, 35% to 40%, 35% to 45%, 35% to 50%, 40% to 45%, 40% to 50%, or 45% to 50%.

The concentration of fat in the dairy product may be about 1%, about 5%, about 10%, about 15%, about 20%, about 25%, about 30%, about 35%, about 40%, about 45%, or about 50%. The concentration of fat in the dairy product may be at least 1%, at least 5%, at least 10%, at least 15%, at least 20%, at least 25%, at least 30%, at least 35%, at least 40%, at least 45%, or at least 50%. The concentration of fat in the dairy product may be at most 1%, at most 5%, at most 10%, at most 15%, at most 20%, at most 25%, at most 30%, at most 35%, at most 40% or at most 45%.

In some embodiments, a coagulated colloid is made from the micelle compositions and micelle-like compositions herein. Coagulated colloid may be generated at a final pH from about 4 to about 6. Coagulated colloid may be generated at a pH from about 4 to about 6. Coagulated colloid may be generated at a final pH of at least 4. Coagulated colloid may be generated at a final pH of at most 6. Coagulated colloid may be generated at a final pH from 4 to 4.5, 4 to 5, 4 to 5.1, 4 to 5.2, 4 to 5.5, 4 to 6, 4.5 to 5, 4.5 to 5.1, 4.5 to 5.2, 4.5 to 5.5, 4.5 to 6, 5 to 5.1, 5 to 5.2, 5 to 5.5, 5 to 6, 5.1 to 5.2, 5.1 to 5.5, 5.1 to 6, 5.2 to 5.5, 5.2 to 6, or 5.5 to 6. Coagulated colloid may be generated at a final pH of about 4, about 4.5, about 5, about 5.1, about 5.2, about 5.5, or about 6. Coagulated colloid may be generated at a final pH of at least 4, at least 4.5, at least 5, at least 5.1, at least 5.2, or at least 5.5. Coagulated colloid may be generated at a final pH of at most 4.5, at most 5, at most 5.1, at most 5.2, at most 5.5, or at most 6.

Treatments for reducing pH and/or achieving a final pH or final pH range, as described herein, may include the addition of an acid such as citric acid, lactic acid, or vinegar (acetic acid). Treatments for reducing pH of a liquid colloid and achieving a final pH or final pH range, as described herein, may include the addition of an acidifying microorganism such as lactic acid bacteria. Exemplary acidifying microorganisms include Lactococci, Streptococci, Lactobacilli, and mixtures of thereof. In some cases, both at least one acid and at least one acidifying microorganism can be added to the liquid colloid to create a coagulated colloid. In some cases, aging and ripening microorganisms (such as bacteria or fungi) can also be added in this step.

In some cases, following acidification, a renneting agent may be added to form a renneted curd (coagulated curd matrix), which may then be used to make cheese. Micelles and micelle-like structures in a liquid colloid, such as milk and also the liquid colloid described herein, can be stable and repel each other in colloidal suspension. In the presence of renneting agents or milk-clotting enzymes, and when acidified, micelles and micelle-like structures can be destabilized and attract each other, and thus coagulate. In the presence of renneting agents or milk-clotting enzymes, crosslinked coagulated curd matrix can be formed. Renneting agents used for curd formation may include chymosin, pepsin A, mucorpepsin, endothiapepsin, or equivalents thereof. Renneting agents may be derived from plants, dairy products, or recombinantly.

In some embodiments, renneted curd is further treated to create a dairy product or dairy-like product (e.g., a cheese or cheese-like product). In some cases, such as a mozzarella product, the renneted curd may be heated and stretched. In certain cases, the renneted curd can be cooked, pressed, and/or aged, such as for brie, camembert, feta, halloumi, gouda, edam, cheddar, manchego, Swiss, colby, muenster, blue cheese, or parmesan type cheese or cheese-like product.

In certain embodiments, coagulated colloid or renneted curd may be treated with hot water for the formation of cheese, such as for mozzarella-type cheese. Hot water treatment may be performed at a temperature from about 50° C. to about 90° C. Hot water treatment may be performed at a temperature of at least 55° C. Hot water treatment may be performed at a temperature of at most 75° C. Hot water treatment may be performed at a temperature from 50° C. to 55° C., 55° C. to 60° C., 55° C. to 65° C., 55° C. to 70° C., 55° C. to 75° C., 60° C. to 65° C., 60° C. to 70° C., 60° C. to 75° C., 65° C. to 70° C., 65° C. to 75° C., 70° C. to 75° C., 75° C. to 80° C., 80° C. to 85° C., or 85° C. to 90° C. Hot water treatment may be performed at a temperature of about 50° C., about 55° C., about 60° C., about 65° C., about 70° C., about 75° C., about 80° C., about 85° C., or about 90° C. Hot water treatment may be performed at a temperature of at least 50° C., at least 55° C., at least 60° C., at least 65° C., at least 70° C., at least 75° C., at least 80° C., or at least 85° C. Hot water treatment may be performed at a temperature of at most 55° C., at most 60° C., at most 65° C., at most 70° C., at most 75° C., at most 80° C., at most 85° C., or at most 90° C. In some cases, after hot water treatment, the product can be stretched into a cheese. In some cases, the cheese can be a mozzarella-like cheese.

Dairy-like compositions (e.g., cheese compositions) formed using the methods described herein may not comprise any animal-obtained components. The dairy-like compositions may not comprise any animal-obtained dairy-based components, such as animal-obtained dairy proteins. The dairy-like compositions may not comprise any whey proteins. The dairy-like compositions may not comprise any beta casein protein. The dairy-like compositions may be a pasta-filata like cheese such as mozzarella cheese. Soft cheeses (or cheese-like products) such as paneer, cream cheese, or cottage cheese may also be formed using the methods described herein. Other types of cheeses (or cheese-like products) such as aged and ripened cheeses may also be formed using the methods described herein, such as brie, camembert, feta, halloumi, gouda, edam, cheddar, manchego, Swiss, colby, muenster, blue cheese, and parmesan.

The texture of a cheese made by methods described herein may be comparable to the texture of a similar type of cheese made using animal-obtained dairy proteins, such as cheese made from animal milk (e.g., dairy cheese). Texture of a cheese may be tested using a trained panel of human subjects or machines such as a texture analyzer.

The hardness of a cheese-like product made by methods as described herein may be comparable to the hardness of a similar type of cheese made using animal-obtained dairy proteins, such as cheese made from animal milk. Hardness of a cheese-like product or a cheese (e.g., a dairy cheese) may be tested using a trained panel of human subjects or machines such as a hardness analyzer.

The taste of a cheese-like product made by methods as described herein may be comparable to a similar type of cheese made using animal-obtained dairy proteins. Taste of a cheese-like product or a cheese may be tested using a trained panel of human subjects.

In some embodiments, the stretch of a cheese made using the methods provided herein and/or comprising the micelle and/or micelle-like compositions described herein may be comparable to an animal-obtained dairy cheese. In some embodiments, the stretch of a cheese made using the methods provided herein and/or comprising the micelle and/or micelle-like compositions described herein may be improved compared to an animal-obtained dairy cheese.

In some embodiments, when the cheese comprises a micelle-like composition of the disclosure (e.g., comprising intra-micellar crosslinking between kappa casein molecules), the stretch of the cheese may be improved as compared to a comparable cheese without the intra-micellar crosslinking between kappa casein molecules.

In some embodiments, the melt of a cheese made using the methods provided herein and/or comprising the micelle and/or micelle-like compositions described herein may be comparable to an animal-obtained dairy cheese. In some embodiments, the melt of a cheese made using the methods provided herein and/or comprising the micelle and/or micelle-like compositions described herein may be improved compared to an animal-obtained dairy cheese.

In some embodiments, when the cheese comprises a micelle-like composition of the disclosure (e.g., comprising intra-micellar crosslinking between kappa casein molecules), the melt of the cheese may be improved as compared to a comparable cheese without the intra-micellar crosslinking between kappa casein molecules.

In some embodiments, when the cheese comprises a micelle-like composition of the disclosure (e.g., comprising intra-micellar crosslinking between kappa casein molecules), the yield of the cheese may be improved as compared to a comparable cheese without the intra-micellar crosslinking between kappa casein molecules.

Cheese-like compositions, as described herein, may have a browning ability which is comparable to a similar type of cheese made using animal-obtained dairy proteins. Cheese-like compositions, as described herein, may have a melting ability which is comparable to a similar type of cheese made using animal-obtained dairy proteins.

In some embodiments, the micelle compositions and micelle-like compositions herein may be used for yogurt formation. In some cases, for yogurt production, the micelle compositions and micelle-like compositions in the form of a liquid colloid may be heat treated. The heat treatment may include treating the liquid colloid at a temperature of about 75° C., 80° C., 85° C., 87° C., 90° C., 92° C., 95° C., or 100° C. The heat treatment may be followed with a cooling step of the liquid colloid.

In certain embodiments, in yogurt production a bacterial culture may be used as a starter culture. Starter bacterial cultures used for yogurt production may be any suitable bacterial cultures. For instance, bacteria known for yogurt generation such as Lactobacillus delbrueckii subsp. bulgaricus, Streptococcus thermophilus, other Lactobacilli, and Bifidobacteria sp. bacteria may be cultured and added to the liquid colloid comprising the one or more recombinant proteins. The bacterial starter culture may be used for the acidification of the micelle compositions and micelle-like compositions herein that are in the form of a liquid colloid. Acidification of a liquid colloid may be continued until a desired consistency of the colloid is achieved. For instance, bacterial acidification may be continued until a desired consistency is reached for the liquid colloid. Bacterial acidification of the liquid colloid may lead to the formation of a coagulated liquid colloid which has a yogurt-like consistency. In various embodiments, a yogurt-like product may be formed using the components and/or methods provided herein.

Bacterial acidification of the liquid colloid in yogurt production may be performed at a temperature from 30° C. to 55° C. In some cases, bacterial acidification of the liquid colloid may be performed at a temperature of at least 30° C. Bacterial acidification of the liquid colloid may be performed at a temperature of at most 55° C. Bacterial acidification of the liquid colloid may be performed at a temperature from 30° C. to 35° C., 30° C. to 40° C., 30° C. to 45° C., 30° C. to 50° C., 30° C. to 55° C., 35° C. to 40° C., 35° C. to 45° C., 35° C. to 50° C., 35° C. to 55° C., 40° C. to 45° C., 40° C. to 50° C., 40° C. to 55° C., 45° C. to 50° C., 45° C. to 55° C., or 50° C. to 55° C. Bacterial acidification of the liquid colloid may be performed at a temperature of about 30° C., about 35° C., about 40° C., about 45° C., about 50° C., or about 55° C. Bacterial acidification of the liquid colloid may be performed at a temperature of at least 30° C., 35° C., 40° C., 45° C. or 50° C. Bacterial acidification of the liquid colloid may be performed at a temperature of at most 35° C., 40° C., 45° C., 50° C., or 55° C. In some cases, bacterial acidification may be performed at a temperature from 30° C. to 55° C. for at least 1 hour. In certain cases, bacterial acidification may be performed at a temperature from 30° C. to 55° C. for at least 2 hours, at least 3 hours, at least 4 hours, at least 5 hours, at least 6 hours, at least 8 hours, at least 10 hours, or at least 12 hours. In various cases, bacterial acidification may be performed at a temperature from 30° C. to 55° C. for at most 1 hour. In some cases, bacterial acidification may be performed at a temperature from 30° C. to 55° C. for at most 2 hours, at most 3 hours, at most 4 hours, at most 5 hours, at most 6 hours, at most 8 hours, at most 10 hours, or at most 12 hours.

Alternatively, bacterial acidification may be performed at a lower temperature from 15° C. to 30° C. Bacterial acidification of the liquid colloid may be performed at a temperature of at least 15° C. Bacterial acidification of the liquid colloid may be performed at a temperature of at most 30° C. Bacterial acidification of the liquid colloid may be performed at a temperature from 15° C. to 17° C., 15° C. to 20° C., 15° C. to 22° C., 15° C. to 25° C., 15° C. to 27° C., 15° C. to 30° C., 17° C. to 20° C., 17° C. to 22° C., 17° C. to 25° C., 17° C. to 27° C., 17° C. to 30° C., 20° C. to 22° C., 20° C. to 25° C., 20° C. to 27° C., 20° C. to 30° C., 22° C. to 25° C., 22° C. to 27° C., 22° C. to 30° C., 25° C. to 27° C., 25° C. to 30° C., or 27° C. to 30° C. Bacterial acidification of the liquid colloid may be performed at a temperature of about 15° C., about 17° C., about 20° C., about 22° C., about 25° C., about 27° C., or about 30° C. Bacterial acidification of the liquid colloid may be performed at a temperature of at least 15° C., at least 17° C., at least 20° C., at least 22° C., at least 25° C., or at least 27° C. Bacterial acidification of the liquid colloid may be performed at a temperature of at most 17° C., at most 20° C., at most 22° C., at most 25° C., at most 27° C., or at most 30° C.

In some cases, bacterial acidification may be performed at a temperature from 15° C. to 30° C. for at least 10 hours. In certain cases, bacterial acidification may be performed at a temperature from 15° C. to 30° C. for at least 10 hours, at least 12 hours, at least 14 hours, at least 16 hours at least 18 hours, at least 20 hours, at least 22 hours, or at least 24 hours. In various cases, bacterial acidification may be performed at a temperature from 15° C. to 30° C. for at most 24 hours. In some cases, bacterial acidification may be performed at a temperature from 15° C. to 30° C. for at most 12 hours, at most 14 hours, at most 16 hours, at most 18 hours, at most 20 hours, at most 22 hours, or at most 24 hours.

Similar to cheese formation, yogurt formation may comprise other components such as sugars, fats, stabilizers, flavoring agents, and colorants.

The concentration of fat in a yogurt product made from a liquid colloid (containing the micelle compositions and micelle-like compositions herein) may be from 0% to 12%. The yogurt product made from the liquid colloid may comprise less than 1% fat or in some cases no fats. The concentration of fat in the yogurt product made from liquid colloid may be at most 12%. The concentration of fat in the yogurt product made from liquid colloid may be from 1% to 2%, 1% to 5%, 1% to 7%, 1% to 10%, 1% to 12%, 2% to 5%, 2% to 7%, 2% to 10%, 2% to 12%, 5% to 7%, 5% to 10%, 5% to 12%, 7% to 10%, 7% to 12%, or 10% to 12%. The concentration of fat in the yogurt product made from liquid colloid may be about 1%, about 2%, about 5%, about 7%, about 10%, or about 12%. The concentration of fat in the yogurt product made from liquid colloid may be at least 1%, at least 2%, at least 5%, at least 7%, or at least 10%. The concentration of fat in the yogurt product made from liquid colloid may be at most 2%, at most 5%, at most 7%, at most 10%, or at most 12%. Fats may be emulsified into liquid colloid (containing the micelle compositions and micelle-like compositions herein)) using a sonication or high-pressure homogenization process. An emulsifier such as soy lecithin or xanthan gum may be used to secure a stable emulsion.

The texture of a yogurt-like product made by methods described herein may be comparable to the texture of a similar type of yogurt made using animal-obtained dairy proteins, such as yogurt made from animal milk. The texture of a yogurt-like product or a yogurt may be tested using a trained panel of human subjects or machines such as a texture analyzer.

The taste of a yogurt-like product made by methods described herein may be comparable to a similar type of yogurt made using animal-obtained dairy proteins. Taste of a yogurt-like product or a yogurt may be tested using a trained panel of human subjects.

The texture of a dairy-like product made by methods described herein may be comparable to the texture of a similar type of dairy product made using animal-obtained dairy proteins, such as ice cream made from animal milk. Texture of a dairy-like product may be tested using a trained panel of human subjects or machines such as a texture analyzer.

The hardness of a dairy-like product made by methods as described herein may be comparable to the hardness of a similar type of dairy product made using animal-obtained dairy proteins, such as ice cream made from animal milk. Hardness of a dairy-like product or a dairy product may be tested using a trained panel of human subjects or machines such as a hardness analyzer.

The taste of a dairy-like product made by methods as described herein may be comparable to a similar type of dairy product made using animal-obtained dairy proteins. Taste of a dairy-like product or a dairy product may be tested using a trained panel of human subjects. One or more dairy-like properties listed herein of a dairy-like product made by methods as described herein may be improved or more desirable when compared to a similar type of dairy product made using animal-obtained dairy proteins.

### Recombinant Expression

One or more proteins used in the formation of micelles and micelle-like compositions, as well as dairy-like compositions, such as any described herein, may be produced recombinantly. In some cases, one or more of alpha-S1, alpha-S2, beta, kappa, and gamma casein are produced recombinantly.

Alpha-S1 and/or alpha-S2 casein can have an amino acid sequence from any species. For example, recombinant alpha casein may have an amino acid sequence of cow, human, sheep, goat, buffalo, bison, horse, or camel alpha casein. Alpha casein nucleotide sequence may be codon-optimized for increased efficiency of production. Exemplary alpha casein protein sequences are provided in Table 1 below. Recombinant alpha casein can be a non-naturally occurring variant or altered form of an alpha casein. Such a variant or altered form can comprise one or more amino acid insertions, deletions, or substitutions relative to a native alpha casein sequence. Such a variant or altered form can have at least 70%, at least 75%, at least 80%, at least 85%, at least 90%, at least 95%, at least 96%, at least 97%, at least 98%, or at least 99% sequence identity to any one of SEQ ID NOs: 1-39 or 64-72. An altered form of an alpha casein protein may be a truncated alpha casein protein relative to a wild-type or native alpha casein protein. The truncation may be a truncation found in nature or an engineered truncation. An altered form of an alpha casein protein may have an N-terminal truncation relative to a wild-type or native alpha casein protein. An altered form of an alpha casein protein may have a C-terminal truncation relative to a wild-type or native alpha casein protein. In some embodiments, a recombinant alpha casein may comprise a mixture of a native alpha casein protein and an altered form (e.g., a truncate) of an alpha casein protein. In some embodiments, a truncated alpha casein protein comprises an amino acid sequence according to any one of SEQ ID NOs: 64-72, or comprises an amino acid sequence having at least 70%, at least 75%, at least 80%, at least 85%, at least 90%, at least 95%, at least 96%, at least 97%, at least 98%, or at least 99% sequence identity to any one of SEQ ID NOs: 64-72.

Kappa casein can have an amino acid sequence from any species. For example, recombinant kappa casein may have an amino acid sequence of cow, human, sheep, goat, buffalo, bison, horse, or camel kappa casein. Kappa casein nucleotide sequence may be codon-optimized for increased efficiency of production. Exemplary kappa casein amino acid sequences are provided in Table 1 below. Recombinant kappa casein can be a non-naturally occurring variant or altered form of a kappa casein. Such a variant or altered form can comprise one or more amino acid insertions, deletions, or substitutions relative to a native kappa sequence. Such a variant or altered form can have at least 70%, at least 75%, at least 80%, at least 85%, at least 90%, at least 95%, at least 96%, at least 97%, at least 98%, or at least 99% sequence identity to any one of SEQ ID NOs: 40-60.

An altered form of a kappa casein protein may be a truncated kappa casein protein relative to a wild-type or native kappa casein protein. The truncation may be a truncation found in nature or an engineered truncation. An altered form of a kappa casein protein may have an N-terminal truncation relative to a wild-type or native kappa casein protein. An altered form of a kappa casein protein may have a C-terminal truncation relative to a wild-type or native kappa casein protein. In some embodiments, a recombinant kappa casein may comprise a mixture of a native kappa casein protein and an altered form (e.g., a truncate) of a kappa casein protein. A recombinant casein (such as an alpha casein or kappa casein) may be recombinantly expressed in a host cell. As used herein, a “host” or “host cell” generally refers to any protein production host selected or genetically modified to produce a desired product. Exemplary hosts include bacteria, yeast, fungi, plant cells, insect cells, and mammalian cells. In some cases, the selected host cell produces an alpha casein or kappa casein that has non-native, reduced, or lacks PTMs. In some cases, a bacterial host cell such as Lactococcus lactis, Bacillus subtilis, or Escherichia coli may be used to produce alpha and/or kappa casein proteins. Other host cells include bacterial hosts such as, but not limited to, Lactococci sp., Lactococcus lactis, Bacillus subtilis, Bacillus amyloliquefaciens, Bacillus lichenformis, Bacillus megaterium, Brevibacillus choshinensis, Mycobacterium smegmatis, Rhodococcus erythropolis, Corynebacterium glutamicum, Lactobacilli sp., Lactobacillus fermentum, Lactobacillus casei, Lactobacillus acidophilus, Lactobacillus plantarum, and Synechocystis sp. 6803.

Alpha and kappa caseins may be produced in the same host cell. Alternatively, alpha and kappa casein may be produced in different host cells. Expression of a target protein can be provided by an expression vector, a plasmid, a nucleic acid integrated into the host genome, or other suitable methods. For example, a vector for expression can include: (a) a promoter element, (b) a signal peptide, (c) a heterologous casein sequence, and (d) a terminator element. In some cases, the one or more expression vectors described herein do not comprise a protein sequence for beta casein (e.g., SEQ ID NOs: 61-63).

Expression vectors that can be used for expression of casein include those containing an expression cassette with elements (a), (b), (c), and (d). In some embodiments, the signal peptide (b) need not be included in the vector. In some cases, a signal peptide may be part of the native signal sequence of the casein protein. For instance, the protein may comprise a native signal sequence as bolded and underlined in any one of SEQ ID NOs: 1, 4, 7, 10, 13, 16, 19, 22, 25, 28, 31, 34, 37, 40, 43, 46, 49, 52, 55, 58, or 61. In some cases, the vector comprises a protein sequence as exemplified in SEQ ID NOs: 1-72. In certain cases, the vector may comprise a mature protein sequence, as exemplified in any one of SEQ ID NOs: 2, 3, 5, 6, 8, 9, 11, 12, 14, 15, 17, 18, 20, 21, 23, 24, 26, 27, 29, 30, 32, 33, 35, 36, 38, 39, 41, 42, 44, 45, 47, 48, 50, 51, 53, 54, 56, 57, 59, 60, 62, or 63 with a heterologous signal sequence. Generally, the expression cassette can be designed to mediate the transcription of the transgene when integrated into the genome of a cognate host microorganism or when present on a plasmid or other replicating vector maintained in a host cell.

To aid in the amplification of the vector prior to transformation into the host microorganism, a replication origin (e) may be contained in the vector. To aid in the selection of microorganism stably transformed with the expression vector, the vector may also include a selection marker (f). The expression vector may also contain a restriction enzyme site (g) that allows for linearization of the expression vector prior to transformation into the host microorganism to facilitate the expression vector's stable integration into the host genome. In some embodiments, the expression vector may contain any subset of the elements (b), (e), (f), and (g), including none of elements (b), (e), (f), and (g). Other expression elements and vector elements can be used in combination or substituted for the elements described herein.

Gram positive bacteria (such as Lactococcus lactis and Bacillus subtilis) may be used to secrete target proteins into the media, and gram-negative bacteria (such as Escherichia coli) may be used to secrete target proteins into periplasm or into the media. In some embodiments, the bacterially expressed proteins expressed may not have any PTMs, which can mean they may not be glycosylated and/or may not be phosphorylated.

Target casein proteins may be expressed and produced in L. lactis both in a nisin-inducible expression system (regulated by PnisA promoter), lactate-inducible expression system (regulated by P170 promoter), or other similar inducible systems, as well as a constitutively expressed system (regulated by P secA promoter), wherein both are in a food-grade selection strain, such as NZ3900 using vector pNZ8149 (lacF gene supplementation/rescue principle). The secretion of functional proteins may be enabled by the signal peptide of Usp45 (SP(usp45)), the major Sec-dependent protein secreted by L. lactis. For example, alpha-S1 casein and kappa casein may be co-expressed or individually expressed in L. lactis using a synthetic operon, where the gene order is kappa casein—alpha-S1 casein.

Bacillus subtilis Design

B. subtilis, unlike L. lactis, has multiple intracellular and extracellular proteases, which may interfere with protein expression. In some embodiments, B. subtilis strains are modified to reduce the type and amount of intracellular and/or extracellular proteases, for example, strains which have deletions for 7 (KO7) and 8 (WB800N) proteases, respectively, may be used.

In order to drive the recombinant protein secretion, the signal peptide of amyQ, alpha-amylase of Clostridium thermocellum may be used. Additionally, native casein signal peptide sequences may be expressed heterologously in B. subtilis. Each casein protein has its own signal peptide sequence and may be used in the system. The signal proteins may be cross-combined with the casein proteins. The pHT01 vector may be used as a transformation and expression shuttle for inducible protein expression in B. subtilis. The vector is based on the strong GA-dependent promoter preceding the groES-groELoperon of B. subtilis, which has been converted into an efficiently controllable (IPTG-inducible) promoter by addition of the lac operator. pHT01 is an E. coli/B. subtilis shuttle vector that provides ampicillin resistance to E. coli and chloramphenicol resistance to B. subtilis.

Untagged and tagged versions of caseins may be expressed, whereby a small peptide tag such as His or StrepII tag, sequence or fusion protein such as GST, MBP, or SUMO is placed N- or C-terminally to casein without the secretion signal peptide. Given secondary structures of kappa, alpha-S1, and alpha-S2 casein, tagging may be less disruptive at N-terminal of kappa casein, whereby alpha-S1 casein can likely be tagged at both termini. However, other tags may be used.

TABLE 1
Sequences

| SEQ | | |
|---|---|---|
| ID | | |
| NO: | Name | Sequence |
| 1 | Bovine Alpha-S1 Casein | MKLLILTCLVAVALARPKHPIKHQGLPQEVLNENL |
| | | LRFFVAPFPEVFGKEKVNELSKDIGSESTEDQAMED |
| | | IKQMEAESISSSEEIVPNSVEQKHIQKEDVPSERYLG |
| | | YLEQLLRLKKYKVPQLEIVPNSAEERLHSMKEGIHA |
| | | QQKEPMIGVNQELAYFYPELFRQFYQLDAYPSGAW |
| | | YYVPLGTQYTDAPSFSDIPNPIGSENSEKTTMPLW |
| 2 | Bovine Alpha-S1 Casein | RPKHPIKHQGLPQEVLNENLLRFFVAPFPEVFGKEK |
| | Mature protein | VNELSKDIGSESTEDQAMEDIKQMEAESISSSEEIVP |
| | | NSVEQKHIQKEDVPSERYLGYLEQLLRLKKYKVPQ |
| | | LEIVPNSAEERLHSMKEGIHAQQKEPMIGVNQELAY |
| | | FYPELFRQFYQLDAYPSGAWYYVPLGTQYTDAPSF |
| | | SDIPNPIGSENSEKTTMPLW |
| 3 | Bovine Alpha-S1 Casein | MRPKHPIKHQGLPQEVLNENLLRFFVAPFPEVFGKE |
| | Mature protein with | KVNELSKDIGSESTEDQAMEDIKQMEAESISSSEEIV |
| | Methionine | PNSVEQKHIQKEDVPSERYLGYLEQLLRLKKYKVP |
| | | QLEIVPNSAEERLHSMKEGIHAQQKEPMIGVNQELA |
| | | YFYPELFRQFYQLDAYPSGAWYYVPLGTQYTDAPS |
| | | FSDIPNPIGSENSEKTTMPLW |
| 4 | Ovine Alpha-S1 Casein | MKLLILTCLVAVALARPKHPIKHQGLSSEVLNENL |
| | | LRFVVAPFPEVFRKENINELSKDIGSESIEDQAMEDA |
| | | KQMKAGSSSSSEEIVPNSAEQKYIQKEDVPSERYLG |
| | | YLEQLLRLKKYNVPQLEIVPKSAEEQLHSMKEGNP |
| | | AHOKQPMIAVNQELAYFYPQLFRQFYQLDAYPSGA |
| | | WYYLPLGTQYTDAPSFSDIPNPIGSENSGKITMPLW |
| 5 | Ovine Alpha-S1 Casein | RPKHPIKHQGLSSEVLNENLLRFVVAPFPEVFRKENI |
| | Mature protein | NELSKDIGSESIEDQAMEDAKQMKAGSSSSSEEIVP |
| | | NSAEQKYIQKEDVPSERYLGYLEQLLRLKKYNVPQ |
| | | LEIVPKSAEEQLHSMKEGNPAHQKQPMIAVNQELA |
| | | YFYPQLFRQFYQLDAYPSGAWYYLPLGTQYTDAPS |
| | | FSDIPNPIGSENSGKITMPLW |
| 6 | Ovine Alpha-S1 Casein | MRPKHPIKHQGLSSEVLNENLLRFVVAPFPEVFRKE |
| | Mature protein with | NINELSKDIGSESIEDQAMEDAKQMKAGSSSSSEEIV |
| | Methionine | PNSAEQKYIQKEDVPSERYLGYLEQLLRLKKYNVP |
| | | QLEIVPKSAEEQLHSMKEGNPAHQKQPMIAVNQEL |
| | | AYFYPQLFRQFYQLDAYPSGAWYYLPLGTQYTDAP |
| | | SFSDIPNPIGSENSGKITMPLW |
| 7 | Caprine Alpha-S1 | MKLLILTCLVAVALARPKHPINHRGLSPEVPNENL |
| | Casein | LRFVVAPFPEVFRKENINELSKDIGSESTEDQAMED |
| | | AKQMKAGSSSSSEEIVPNSAEQKYIQKEDVPSERYL |
| | | GYLEQLLRLKKYNVPQLEIVPKSAEEQLHSMKEGN |
| | | PAHQKQPMIAVNQELAYFYPQLFRQFYQLDAYPSG |
| | | AWYYLPLGTQYTDAPSFSDIPNPIGSENSGKTTMPL |
| | | W |
| 8 | Caprine Alpha-S1 | RPKHPINHRGLSPEVPNENLLRFVVAPFPEVFRKENI |
| | Casein Mature Protein | NELSKDIGSESTEDQAMEDAKQMKAGSSSSSEEIVP |
| | | NSAEQKYIQKEDVPSERYLGYLEQLLRLKKYNVPQ |
| | | LEIVPKSAEEQLHSMKEGNPAHQKQPMIAVNQELA |
| | | YFYPQLFRQFYQLDAYPSGAWYYLPLGTQYTDAPS |
| | | FSDIPNPIGSENSGKTTMPLW |
| 9 | Caprine Alpha-S1 | MRPKHPINHRGLSPEVPNENLLRFVVAPFPEVFRKE |
| | Casein Mature Protein | NINELSKDIGSESTEDQAMEDAKQMKAGSSSSSEEI |
| | with Methionine | VPNSAEQKYIQKEDVPSERYLGYLEQLLRLKKYNV |
| | | PQLEIVPKSAEEQLHSMKEGNPAHQKQPMIAVNQE |
| | | LAYFYPQLFRQFYQLDAYPSGAWYYLPLGTQYTD |
| | | APSFSDIPNPIGSENSGKTTMPLW |
| 10 | Buffalo Alpha-S1 | MKLLILTCLVAVALARPKQPIKHQGLPQGVLNEN |
| | Casein | LLRFFVAPFPEVFGKEKVNELSTDIGSESTEDQAME |
| | | DIKQMEAESISSSEEIVPISVEQKHIQKEDVPSERYLG |
| | | YLEQLLRLKKYNVPQLEIVPNLAEEQLHSMKEGIH |
| | | AQQKEPMIGVNQELAYFYPQLFRQFYQLDAYPSGA |
| | | WYYVPLGTQYPDAPSFSDIPNPIGSENSEKTTMPLW |
| 11 | Buffalo Alpha-S1 | RPKQPIKHQGLPQGVLNENLLRFFVAPFPEVFGKEK |
| | Casein Mature Protein | VNELSTDIGSESTEDQAMEDIKQMEAESISSSEEIVPI |
| | | SVEQKHIQKEDVPSERYLGYLEQLLRLKKYNVPQL |
| | | EIVPNLAEEQLHSMKEGIHAQQKEPMIGVNQELAY |
| | | FYPQLFRQFYQLDAYPSGAWYYVPLGTQYPDAPSF |
| | | SDIPNPIGSENSEKTTMPLW |
| 12 | Buffalo Alpha-S1 | MRPKQPIKHQGLPQGVLNENLLRFFVAPFPEVFGKE |
| | Casein Mature Protein | KVNELSTDIGSESTEDQAMEDIKQMEAESISSSEEIV |
| | with Methionine | PISVEQKHIQKEDVPSERYLGYLEQLLRLKKYNVPQ |
| | | LEIVPNLAEEQLHSMKEGIHAQQKEPMIGVNQELA |
| | | YFYPQLFRQFYQLDAYPSGAWYYVPLGTQYPDAPS |
| | | FSDIPNPIGSENSEKTTMPLW |
| 13 | Equine Alpha-S1 Casein | MKLLILTCLVAVALARPKLPHRQPEIIQNEQDSRE |
| | | KVLKERKFPSFALEYINELNRQRELLKEKQKDEHKE |
| | | YLIEDPEQQESSSTSSSEEVVPINTEQKRIPREDMLY |
| | | QHTLEQLRRLSKYNQLQLQAIHAQEQLIRMKENSQ |
| | | RKPMRVVNQEQAYFYLEPFQPSYQLDVYPYAAWF |
| | | HPAQIMQHVAYSPFHDTAKLIASENSEKTDIIPEW |
| 14 | Equine Alpha-S1 Casein | RPKLPHRQPEIIQNEQDSREKVLKERKFPSFALEYIN |
| | Mature Protein | ELNRQRELLKEKQKDEHKEYLIEDPEQQESSSTSSS |
| | | EEVVPINTEQKRIPREDMLYQHTLEQLRRLSKYNQL |
| | | QLQAIHAQEQLIRMKENSQRKPMRVVNQEQAYFY |
| | | LEPFQPSYQLDVYPYAAWFHPAQIMQHVAYSPFHD |
| | | TAKLIASENSEKTDIIPEW |
| 15 | Equine Alpha-S1 Casein | MRPKLPHRQPEIIQNEQDSREKVLKERKFPSFALEYI |
| | Mature Protein with | NELNRQRELLKEKQKDEHKEYLIEDPEQQESSSTSS |
| | Methionine | SEEVVPINTEQKRIPREDMLYQHTLEQLRRLSKYNQ |
| | | LQLQAIHAQEQLIRMKENSQRKPMRVVNQEQAYF |
| | | YLEPFQPSYQLDVYPYAAWFHPAQIMQHVAYSPFH |
| | | DTAKLIASENSEKTDIIPEW |
| 16 | Camel Alpha-S1 Casein | MKLLILTCLVAVALARPKYPLRYPEVFQNEPDSIE |
| | | EVLNKRKILELAVVSPIQFRQENIDELKDTRNEPTED |
| | | HIMEDTERKESGSSSSEEVVSSTTEQKDILKEDMPS |
| | | QRYLEELHRLNKYKLLQLEAIRDQKLIPRVKLSSHP |
| | | YLEQLYRINEDNHPQLGEPVKVVTQEQAYFHLEPFP |
| | | QFFQLGASPYVAWYYPPQVMQYIAHPSSYDTPEGI |
| | | ASEDGGKTDVMPQWW |
| 17 | Camel Alpha-S1 Casein | RPKYPLRYPEVFQNEPDSIEEVLNKRKILELAVVSPI |
| | Mature Protein | QFRQENIDELKDTRNEPTEDHIMEDTERKESGSSSSE |
| | | EVVSSTTEQKDILKEDMPSQRYLEELHRLNKYKLL |
| | | QLEAIRDQKLIPRVKLSSHPYLEQLYRINEDNHPQL |
| | | GEPVKVVTQEQAYFHLEPFPQFFQLGASPYVAWYY |
| | | PPQVMQYIAHPSSYDTPEGIASEDGGKTDVMPQW |
| | | W |
| 18 | Camel Alpha-S1 Casein | MRPKYPLRYPEVFQNEPDSIEEVLNKRKILELAVVS |
| | Mature Protein with | PIQFRQENIDELKDTRNEPTEDHIMEDTERKESGSSS |
| | Methionine | SEEVVSSTTEQKDILKEDMPSQRYLEELHRLNKYKL |
| | | LQLEAIRDQKLIPRVKLSSHPYLEQLYRINEDNHPQL |
| | | GEPVKVVTQEQAYFHLEPFPQFFQLGASPYVAWYY |
| | | PPQVMQYIAHPSSYDTPEGIASEDGGKTDVMPQW |
| | | W |
| 19 | Human Alpha-S1 | MRLLILTCLVAVALARPKLPLRYPERLQNPSESSEP |
| | Casein | IPLESREEYMNGMNRQRNILREKQTDEIKDTRNEST |
| | | QNCVVAEPEKMESSISSSSEEMSLSKCAEQFCRLNE |
| | | YNQLQLQAAHAQEQIRRMNENSHVQVPFQQLNQL |
| | | AAYPYAVWYYPQIMQYVPFPPFSDISNPTAHENYE |
| | | KNNVMLQW |
| 20 | Human Alpha-S1 | RPKLPLRYPERLQNPSESSEPIPLESREEYMNGMNR |
| | Casein Mature Protein | QRNILREKQTDEIKDTRNESTQNCVVAEPEKMESSI |
| | | SSSSEEMSLSKCAEQFCRLNEYNQLQLQAAHAQEQI |
| | | RRMNENSHVQVPFQQLNQLAAYPYAVWYYPQIMQ |
| | | YVPFPPFSDISNPTAHENYEKNNVMLQW |
| 21 | Human Alpha-S1 | MRPKLPLRYPERLQNPSESSEPIPLESREEYMNGMN |
| | Casein Mature Protein | RQRNILREKQTDEIKDTRNESTQNCVVAEPEKMESS |
| | with Methionine | ISSSSEEMSLSKCAEQFCRLNEYNQLQLQAAHAQEQ |
| | | IRRMNENSHVQVPFQQLNQLAAYPYAVWYYPQIM |
| | | QYVPFPPFSDISNPTAHENYEKNNVMLQW |
| 22 | Bovine Alpha-S2 Casein | MKFFIFTCLLAVALAKNTMEHVSSSEESIISQETYK |
| | | QEKNMAINPSKENLCSTFCKEVVRNANEEEYSIGSS |
| | | SEESAEVATEEVKITVDDKHYQKALNEINQFYQKFP |
| | | QYLQYLYQGPIVLNPWDQVKRNAVPITPTLNREQL |
| | | STSEENSKKTVDMESTEVFTKKTKLTEEEKNRLNFL |
| | | KKISQRYQKFALPQYLKTVYQHQKAMKPWIQPKT |
| | | KVIPYVRYL |
| 23 | Bovine Alpha-S2 Casein | KNTMEHVSSSEESIISQETYKQEKNMAINPSKENLC |
| | Mature protein | STFCKEVVRNANEEEYSIGSSSEESAEVATEEVKITV |
| | | DDKHYQKALNEINQFYQKFPQYLQYLYQGPIVLNP |
| | | WDQVKRNAVPITPTLNREQLSTSEENSKKTVDMES |
| | | TEVFTKKTKLTEEEKNRLNFLKKISQRYQKFALPQY |
| | | LKTVYQHQKAMKPWIQPKTKVIPYVRYL |
| 24 | Bovine Alpha-S2 Casein | MKNTMEHVSSSEESIISQETYKQEKNMAINPSKENL |
| | Mature protein with | CSTFCKEVVRNANEEEYSIGSSSEESAEVATEEVKIT |
| | Methionine | VDDKHYQKALNEINQFYQKFPQYLQYLYQGPIVLN |
| | | PWDQVKRNAVPITPTLNREQLSTSEENSKKTVDME |
| | | STEVFTKKTKLTEEEKNRLNFLKKISQRYQKFALPQ |
| | | YLKTVYQHQKAMKPWIQPKTKVIPYVRYL |
| 25 | Ovine Alpha-S2 Casein | MKFFIFTCLLAVALAKHKMEHVSSSEEPINISQEIY |
| | | KQEKNMAIHPRKEKLCTTSCEEVVRNADEEEYSIRS |
| | | SSEESAEVAPEEVKITVDDKHYQKALNEINQFYQKF |
| | | PQYLQYLYQGPIVLNPWDQVKRNAGPFTPTVNREQ |
| | | LSTSEENSKKTIDMESTEVFTKKTKLTEEEKNRLNF |
| | | LKKISQYYQKFAWPQYLKTVDQHQKAMKPWTQP |
| | | KTNAIPYVRYL |
| 26 | Ovine Alpha-S2 Casein | KHKMEHVSSSEEPINISQEIYKQEKNMAIHPRKEKL |
| | Mature protein | CTTSCEEVVRNADEEEYSIRSSSEESAEVAPEEVKIT |
| | | VDDKHYQKALNEINQFYQKFPQYLQYLYQGPIVLN |
| | | PWDQVKRNAGPFTPTVNREQLSTSEENSKKTIDME |
| | | STEVFTKKTKLTEEEKNRLNFLKKISQYYQKFAWP |
| | | QYLKTVDQHQKAMKPWTQPKTNAIPYVRYL |
| 27 | Ovine Alpha-S2 Casein | MKHKMEHVSSSEEPINISQEIYKQEKNMAIHPRKEK |
| | Mature protein with | LCTTSCEEVVRNADEEEYSIRSSSEESAEVAPEEVKI |
| | Methionine | TVDDKHYQKALNEINQFYQKFPQYLQYLYQGPIVL |
| | | NPWDQVKRNAGPFTPTVNREQLSTSEENSKKTIDM |
| | | ESTEVFTKKTKLTEEEKNRLNFLKKISQYYQKFAWP |
| | | QYLKTVDQHQKAMKPWTQPKTNAIPYVRYL |
| 28 | Caprine Alpha-S2 | MKFFIFTCLLAVALAKHKMEHVSSSEEPINIFQEIY |
| | Casein | KQEKNMAIHPRKEKLCTTSCEEVVRNANEEEYSIRS |
| | | SSEESAEVAPEEIKITVDDKHYQKALNEINQFYQKF |
| | | PQYLQYPYQGPIVLNPWDQVKRNAGPFTPTVNREQ |
| | | LSTSEENSKKTIDMESTEVFTKKTKLTEEEKNRLNF |
| | | LKKISQYYQKFAWPQYLKTVDQHQKAMKPWTQP |
| | | KTNAIPYVRYL |
| 29 | Caprine Alpha-S2 | KHKMEHVSSSEEPINIFQEIYKQEKNMAIHPRKEKL |
| | Casein Mature Protein | CTTSCEEVVRNANEEEYSIRSSSEESAEVAPEEIKITV |
| | | DDKHYQKALNEINQFYQKFPQYLQYPYQGPIVLNP |
| | | WDQVKRNAGPFTPTVNREQLSTSEENSKKTIDMES |
| | | TEVFTKKTKLTEEEKNRLNFLKKISQYYQKFAWPQ |
| | | YLKTVDQHQKAMKPWTQPKTNAIPYVRYL |
| 30 | Caprine Alpha-S2 | MKHKMEHVSSSEEPINIFQEIYKQEKNMAIHPRKEK |
| | Casein Mature Protein | LCTTSCEEVVRNANEEEYSIRSSSEESAEVAPEEIKIT |
| | with Methionine | VDDKHYQKALNEINQFYQKFPQYLQYPYQGPIVLN |
| | | PWDQVKRNAGPFTPTVNREQLSTSEENSKKTIDME |
| | | STEVFTKKTKLTEEEKNRLNFLKKISQYYQKFAWP |
| | | QYLKTVDQHQKAMKPWTQPKTNAIPYVRYL |
| 31 | Buffalo Alpha-S2 | MKFFIFTCLLAVALAKHTMEHVSSSEESIISQETYK |
| | Casein | QEKNMAIHPSKENLCSTFCKEVIRNANEEEYSIGSSS |
| | | EESAEVATEEVKITVDDKHYQKALNEINQFYQKFP |
| | | QYLQYLYQGPIVLNPWDQVKRNAVPITPTLNREQL |
| | | STSEENSKKTVDMESTEVFTKKTKLTEEDKNRLNFL |
| | | KKISQHYQKFAWPQYLKTVYQYQKAMKPWTQPK |
| | | TNVIPYVRYL |
| 32 | Buffalo Alpha-S2 | KHTMEHVSSSEESIISQETYKQEKNMAIHPSKENLC |
| | Casein Mature Protein | STFCKEVIRNANEEEYSIGSSSEESAEVATEEVKITV |
| | | DDKHYQKALNEINQFYQKFPQYLQYLYQGPIVLNP |
| | | WDQVKRNAVPITPTLNREQLSTSEENSKKTVDMES |
| | | TEVFTKKTKLTEEDKNRLNFLKKISQHYQKFAWPQ |
| | | YLKTVYQYQKAMKPWTQPKTNVIPYVRYL |
| 33 | Buffalo Alpha-S2 | MKHTMEHVSSSEESIISQETYKQEKNMAIHPSKENL |
| | Casein Mature Protein | CSTFCKEVIRNANEEEYSIGSSSEESAEVATEEVKIT |
| | with Methionine | VDDKHYQKALNEINQFYQKFPQYLQYLYQGPIVLN |
| | | PWDQVKRNAVPITPTLNREQLSTSEENSKKTVDME |
| | | STEVFTKKTKLTEEDKNRLNFLKKISQHYQKFAWP |
| | | QYLKTVYQYQKAMKPWTQPKTNVIPYVRYL |
| 34 | Equine Alpha-S2 Casein | MKFFIFTCLLAVALAKHNMEHRSSSEDSVNISQEK |
| | | FKQEKYVVIPTSKESICSTSCEEATRNINEMESAKFP |
| | | TEREEKEVEEKHHLKQLNKINQFYEKLNFLQYLQA |
| | | LRQPRIVLTPWDQTKTGDSPFIPIVNTEQLFTSEEIPK |
| | | KTVDMESTEVVTEKTELTEEEKNYLKLLYYEKFTL |
| | | PQYFKIVRQHQTTMDPRSHRKTNSYQIIPVLRYF |
| 35 | Equine Alpha-S2 Casein | KHNMEHRSSSEDSVNISQEKFKQEKYVVIPTSKESIC |
| | Mature Protein | STSCEEATRNINEMESAKFPTEREEKEVEEKHHLKQ |
| | | LNKINQFYEKLNFLQYLQALRQPRIVLTPWDQTKT |
| | | GDSPFIPIVNTEQLFTSEEIPKKTVDMESTEVVTEKT |
| | | ELTEEEKNYLKLLYYEKFTLPQYFKIVRQHQTTMD |
| | | PRSHRKTNSYQIIPVLRYF |
| 36 | Equine Alpha-S2 Casein | MKHNMEHRSSSEDSVNISQEKFKQEKYVVIPTSKES |
| | Mature Protein with | ICSTSCEEATRNINEMESAKFPTEREEKEVEEKHHL |
| | Methionine | KQLNKINQFYEKLNFLQYLQALRQPRIVLTPWDQT |
| | | KTGDSPFIPIVNTEQLFTSEEIPKKTVDMESTEVVTE |
| | | KTELTEEEKNYLKLLYYEKFTLPQYFKIVRQHQTT |
| | | MDPRSHRKTNSYQIIPVLRYF |
| 37 | Camel Alpha-S2 Casein | MKFFIFTCLLAVVLAKHEMDQGSSSEESINVSQQK |
| | | FKQVKKVAIHPSKEDICSTFCEEAVRNIKEVESAEVP |
| | | TENKISQFYQKWKFLQYLQALHQGQIVMNPWDQG |
| | | KTRAYPFIPTVNTEQLSISEESTEVPTEESTEVFTKKT |
| | | ELTEEEKDHQKFLNKIYQYYQTFLWPEYLKTVYQY |
| | | QKTMTPWNHIKRYF |
| 38 | Camel Alpha-S2 Casein | KHEMDQGSSSEESINVSQQKFKQVKKVAIHPSKEDI |
| | Mature Protein | CSTFCEEAVRNIKEVESAEVPTENKISQFYQKWKFL |
| | | QYLQALHQGQIVMNPWDQGKTRAYPFIPTVNTEQL |
| | | SISEESTEVPTEESTEVFTKKTELTEEEKDHQKFLNK |
| | | IYQYYQTFLWPEYLKTVYQYQKTMTPWNHIKRYF |
| 39 | Camel Alpha-S2 Casein | MKHEMDQGSSSEESINVSQQKFKQVKKVAIHPSKE |
| | Mature Protein with | DICSTFCEEAVRNIKEVESAEVPTENKISQFYQKWK |
| | Methionine | FLQYLQALHQGQIVMNPWDQGKTRAYPFIPTVNTE |
| | | QLSISEESTEVPTEESTEVFTKKTELTEEEKDHQKFL |
| | | NKIYQYYQTFLWPEYLKTVYQYQKTMTPWNHIKR |
| | | YF |
| 40 | Bovine Kappa Casein | MMKSFFLVVTILALTLPFLGAQEQNQEQPIRCEK |
| | | DERFFSDKIAKYIPIQYVLSRYPSYGLNYYQQKPVA |
| | | LINNQFLPYPYYAKPAAVRSPAQILQWQVLSNTVP |
| | | AKSCQAQPTTMARHPHPHLSFMAIPPKKNQDKTEIP |
| | | TINTIASGEPTSTPTTEAVESTVATLEDSPEVIESPPEI |
| | | NTVQVTSTAV |
| 41 | Bovine Kappa Casein | QEQNQEQPIRCEKDERFFSDKIAKYIPIQYVLSRYPS |
| | Mature protein | YGLNYYQQKPVALINNQFLPYPYYAKPAAVRSPAQ |
| | | ILQWQVLSNTVPAKSCQAQPTTMARHPHPHLSFMA |
| | | IPPKKNQDKTEIPTINTIASGEPTSTPTTEAVESTVAT |
| | | LEDSPEVIESPPEINTVQVTSTAV |
| 42 | Bovine Kappa Casein | MQEQNQEQPIRCEKDERFFSDKIAKYIPIQYVLSRYP |
| | Mature protein with | SYGLNYYQQKPVALINNQFLPYPYYAKPAAVRSPA |
| | Methionine | QILQWQVLSNTVPAKSCQAQPTTMARHPHPHLSFM |
| | | AIPPKKNQDKTEIPTINTIASGEPTSTPTTEAVESTVA |
| | | TLEDSPEVIESPPEINTVQVTSTAV |
| 43 | Ovine Kappa Casein | MMKSFFLVVTILALTLPFLGAQEQNQEQRICCEK |
| | | DERFFDDKIAKYIPIQYVLSRYPSYGLNYYQQRPVA |
| | | LINNQFLPYPYYAKPVAVRSPAQTLQWQVLPNAVP |
| | | AKSCQDQPTAMARHPHPHLSFMAIPPKKDQDKTEI |
| | | PAINTIASAEPTVHSTPTTEAVVNAVDNPEASSESIA |
| | | SAPETNTAQVTSTEV |
| 44 | Ovine Kappa Casein | QEQNQEQRICCEKDERFFDDKIAKYIPIQYVLSRYPS |
| | Mature protein | YGLNYYQQRPVALINNQFLPYPYYAKPVAVRSPAQ |
| | | TLQWQVLPNAVPAKSCQDQPTAMARHPHPHLSFM |
| | | AIPPKKDQDKTEIPAINTIASAEPTVHSTPTTEAVVN |
| | | AVDNPEASSESIASAPETNTAQVTSTEV |
| 45 | Ovine Kappa Casein | MQEQNQEQRICCEKDERFFDDKIAKYIPIQYVLSRY |
| | Mature protein with | PSYGLNYYQQRPVALINNQFLPYPYYAKPVAVRSP |
| | Methionine | AQTLQWQVLPNAVPAKSCQDQPTAMARHPHPHLS |
| | | FMAIPPKKDQDKTEIPAINTIASAEPTVHSTPTTEAV |
| | | VNAVDNPEASSESIASAPETNTAQVTSTEV |
| 46 | Caprine Kappa Casein | MMKSFFLVVTILALTLPFLGAQEQNQEQPICCEK |
| | | DERFFDDKIAKYIPIQYVLSRYPSYGLNYYQQRPVA |
| | | LINNQFLPYPYYAKPVAVRSPAQTLQWQVLPNTVP |
| | | AKSCQDQPTTLARHPHPHLSFMAIPPKKDQDKTEV |
| | | PAINTIASAEPTVHSTPTTEAIVNTVDNPEASSESIAS |
| | | ASETNTAQVTSTEV |
| 47 | Caprine Kappa Casein | QEQNQEQPICCEKDERFFDDKIAKYIPIQYVLSRYPS |
| | Mature Protein | YGLNYYQQRPVALINNQFLPYPYYAKPVAVRSPAQ |
| | | TLQWQVLPNTVPAKSCQDQPTTLARHPHPHLSFMA |
| | | IPPKKDQDKTEVPAINTIASAEPTVHSTPTTEAIVNT |
| | | VDNPEASSESIASASETNTAQVTSTEV |
| 48 | Caprine Kappa Casein | MQEQNQEQPICCEKDERFFDDKIAKYIPIQYVLSRY |
| | Mature Protein with | PSYGLNYYQQRPVALINNQFLPYPYYAKPVAVRSP |
| | Methionine | AQTLQWQVLPNTVPAKSCQDQPTTLARHPHPHLSF |
| | | MAIPPKKDQDKTEVPAINTIASAEPTVHSTPTTEAIV |
| | | NTVDNPEASSESIASASETNTAQVTSTEV |
| 49 | Buffalo Kappa Casein | MMKSFFLVVTILALTLPFLGAQEQNQEQPIRCEKE |
| | | ERFFNDKIAKYIPIQYVLSRYPSYGLNYYQQKPVALI |
| | | NNQFLPYPYYAKPAAVRSPAQILQWQVLPNTVPAK |
| | | SCQAQPTTMTRHPHPHLSFMAIPPKKNQDKTEIPTI |
| | | NTIVSVEPTSTPTTEAIENTVATLEASSEVIESVPETN |
| | | TAQVTSTVV |
| 50 | Buffalo Kappa Casein | QEQNQEQPIRCEKEERFFNDKIAKYIPIQYVLSRYPS |
| | Mature Protein | YGLNYYQQKPVALINNQFLPYPYYAKPAAVRSPAQ |
| | | ILQWQVLPNTVPAKSCQAQPTTMTRHPHPHLSFMA |
| | | IPPKKNQDKTEIPTINTIVSVEPTSTPTTEAIENTVAT |
| | | LEASSEVIESVPETNTAQVTSTVV |
| 51 | Buffalo Kappa Casein | MQEQNQEQPIRCEKEERFFNDKIAKYIPIQYVLSRYP |
| | Mature Protein with | SYGLNYYQQKPVALINNQFLPYPYYAKPAAVRSPA |
| | Methionine | QILQWQVLPNTVPAKSCQAQPTTMTRHPHPHLSFM |
| | | AIPPKKNQDKTEIPTINTIVSVEPTSTPTTEAIENTVA |
| | | TLEASSEVIESVPETNTAQVTSTVV |
| 52 | Equine Kappa Casein | MKSFFLVVNILALTLPFLGAEVQNQEQPTCHKND |
| | | ERFFDLKTVKYIPIYYVLNSSPRYEPIYYQHRLALLI |
| | | NNQHMPYQYYARPAAVRPHVQIPQWQVLPNIYPST |
| | | VVRHPCPHPSFIAIPPKKLQEITVIPKINTIATVEPTPIP |
| | | TPEPTVNNAVIPDASSEFIIASTPETTTVPVTSPVVQK |
| | | L |
| 53 | Equine Kappa Casein | EVQNQEQPTCHKNDERFFDLKTVKYIPIYYVLNSSP |
| | Mature Protein | RYEPIYYQHRLALLINNQHMPYQYYARPAAVRPHV |
| | | QIPQWQVLPNIYPSTVVRHPCPHPSFIAIPPKKLQEIT |
| | | VIPKINTIATVEPTPIPTPEPTVNNAVIPDASSEFIIAST |
| | | PETTTVPVTSPVVQKL |
| 54 | Equine Kappa Casein | MEVQNQEQPTCHKNDERFFDLKTVKYIPIYYVLNS |
| | Mature Protein with | SPRYEPIYYQHRLALLINNQHMPYQYYARPAAVRP |
| | Methionine | HVQIPQWQVLPNIYPSTVVRHPCPHPSFIAIPPKKLQ |
| | | EITVIPKINTIATVEPTPIPTPEPTVNNAVIPDASSEFII |
| | | ASTPETTTVPVTSPVVQKL |
| 55 | Camel Kappa Casein | MKSFFLVVTILALTLPFLGAEVQNQEQPTCFEKVE |
| | | RLLNEKTVKYFPIQFVQSRYPSYGINYYQHRLAVPI |
| | | NNQFIPYPNYAKPVAIRLHAQIPQCQALPNIDPPTVE |
| | | RRPRPRPSFIAIPPKKTQDKTVNPAINTVATVEPPVIP |
| | | TAEPAVNTVVIAEASSEFITTSTPETTTVQITSTEI |
| 56 | Camel Kappa Casein | EVQNQEQPTCFEKVERLLNEKTVKYFPIQFVQSRYP |
| | Mature Protein | SYGINYYQHRLAVPINNQFIPYPNYAKPVAIRLHAQI |
| | | PQCQALPNIDPPTVERRPRPRPSFIAIPPKKTQDKTV |
| | | NPAINTVATVEPPVIPTAEPAVNTVVIAEASSEFITTS |
| | | TPETTTVQITSTEI |
| 57 | Camel Kappa Casein | MEVQNQEQPTCFEKVERLLNEKTVKYFPIQFVQSR |
| | Mature Protein with | YPSYGINYYQHRLAVPINNQFIPYPNYAKPVAIRLH |
| | Methionine | AQIPQCQALPNIDPPTVERRPRPRPSFIAIPPKKTQDK |
| | | TVNPAINTVATVEPPVIPTAEPAVNTVVIAEASSEFIT |
| | | TSTPETTTVQITSTEI |
| 58 | Human Kappa Casein | MKSFLLVVNALALTLPFLAVEVQNQKQPACHEN |
| | | DERPFYQKTAPYVPMYYVPNSYPYYGTNLYQRRP |
| | | AIAINNPYVPRTYYANPAVVRPHAQIPQRQYLPNSH |
| | | PPTVVRRPNLHPSFIAIPPKKIQDKIIIPTINTIATVEPT |
| | | PAPATEPTVDSVVTPEAFSESIITSTPETTTVAVTPPT |
| | | A |
| 59 | Human Kappa Casein | EVQNQKQPACHENDERPFYQKTAPYVPMYYVPNS |
| | Mature Protein | YPYYGTNLYQRRPAIAINNPYVPRTYYANPAVVRP |
| | | HAQIPQRQYLPNSHPPTVVRRPNLHPSFIAIPPKKIQ |
| | | DKIIIPTINTIATVEPTPAPATEPTVDSVVTPEAFSESII |
| | | TSTPETTTVAVTPPTA |
| 60 | Human Kappa Casein | EVQNQKQPACHENDERPFYQKTAPYVPMYYVPNS |
| | Mature Protein with | YPYYGTNLYQRRPAIAINNPYVPRTYYANPAVVRP |
| | Methionine | HAQIPQRQYLPNSHPPTVVRRPNLHPSFIAIPPKKIQ |
| | | DKIIIPTINTIATVEPTPAPATEPTVDSVVTPEAFSESII |
| | | TSTPETTTVAVTPPTA |
| 61 | Bovine Beta Casein | MKVLILACLVALALARELEELNVPGEIVESLSSSEE |
| | | SITRINKKIEKFQSEEQQQTEDELQDKIHPFAQTQSL |
| | | VYPFPGPIPNSLPQNIPPLTQTPVVVPPFLQPEVMGV |
| | | SKVKEAMAPKHKEMPFPKYPVEPFTESQSLTLTDV |
| | | ENLHLPLPLLQSWMHQPHQPLPPTVMFPPQSVLSLS |
| | | QSKVLPVPQKAVPYPQRDMPIQAFLLYQEPVLGPV |
| | | RGPFPIIV |
| 62 | Bovine Beta Casein | RELEELNVPGEIVESLSSSEESITRINKKIEKFQSEEQ |
| | Mature Protein | QQTEDELQDKIHPFAQTQSLVYPFPGPIPNSLPQNIP |
| | | PLTQTPVVVPPFLQPEVMGVSKVKEAMAPKHKEM |
| | | PFPKYPVEPFTESQSLTLTDVENLHLPLPLLQSWMH |
| | | QPHQPLPPTVMFPPQSVLSLSQSKVLPVPQKAVPYP |
| | | QRDMPIQAFLLYQEPVLGPVRGPFPIIV |
| 63 | Bovine Beta Casein | MRELEELNVPGEIVESLSSSEESITRINKKIEKFQSEE |
| | Mature Protein with | QQQTEDELQDKIHPFAQTQSLVYPFPGPIPNSLPQNI |
| | Methionine | PPLTQTPVVVPPFLQPEVMGVSKVKEAMAPKHKE |
| | | MPFPKYPVEPFTESQSLTLTDVENLHLPLPLLQSWM |
| | | HQPHQPLPPTVMFPPQSVLSLSQSKVLPVPQKAVPY |
| | | PQRDMPIQAFLLYQEPVLGPVRGPFPIIV |
| 64 | Bovine Alpha-S1 Casein | FFVAPFPEVFGKEKVNELSKDIGSESTEDQAMEDIK |
| | (B. Taurus) aa 23-199 | QMEAESISSSEEIVPNSVEQKHIQKEDVPSERYLGYL |
| | | EQLLRLKKYKVPQLEIVPNSAEERLHSMKEGIHAQ |
| | | QKEPMIGVNQELAYFYPELFRQFYQLDAYPSGAWY |
| | | YVPLGTQYTDAPSFSDIPNPIGSENSGKTTMPLW |
| 65 | Bovine Alpha-S1 Casein | MFFVAPFPEVFGKEKVNELSKDIGSESTEDQAMEDI |
| | (B. Taurus) aa 23-199 | KQMEAESISSSEEIVPNSVEQKHIQKEDVPSERYLGY |
| | with Methionine | LEQLLRLKKYKVPQLEIVPNSAEERLHSMKEGIHAQ |
| | | QKEPMIGVNQELAYFYPELFRQFYQLDAYPSGAWY |
| | | YVPLGTQYTDAPSFSDIPNPIGSENSGKTTMPLW |
| 66 | Bovine Alpha-S1 Casein | FVAPFPEVFGKEKVNELSKDIGSESTEDQAMEDIKQ |
| | (B. Taurus) aa 24-199 | MEAESISSSEEIVPNSVEQKHIQKEDVPSERYLGYLE |
| | | QLLRLKKYKVPQLEIVPNSAEERLHSMKEGIHAQQ |
| | | KEPMIGVNQELAYFYPELFRQFYQLDAYPSGAWYY |
| | | VPLGTQYTDAPSFSDIPNPIGSENSGKTTMPLW |
| 67 | Bovine Alpha-S1 Casein | MFVAPFPEVFGKEKVNELSKDIGSESTEDQAMEDIK |
| | (B. Taurus) aa 24-199 | QMEAESISSSEEIVPNSVEQKHIQKEDVPSERYLGYL |
| | with Methionine | EQLLRLKKYKVPQLEIVPNSAEERLHSMKEGIHAQ |
| | | QKEPMIGVNQELAYFYPELFRQFYQLDAYPSGAWY |
| | | YVPLGTQYTDAPSFSDIPNPIGSENSGKTTMPLW |
| 68 | Bovine Alpha-S1 Casein | VAPFPEVFGKEKVNELSKDIGSESTEDQAMEDIKQ |
| | (B. Taurus) aa 25-199 | MEAESISSSEEIVPNSVEQKHIQKEDVPSERYLGYLE |
| | | QLLRLKKYKVPQLEIVPNSAEERLHSMKEGIHAQQ |
| | | KEPMIGVNQELAYFYPELFRQFYQLDAYPSGAWYY |
| | | VPLGTQYTDAPSFSDIPNPIGSENSGKTTMPLW |
| 69 | Bovine Alpha-S1 Casein | MVAPFPEVFGKEKVNELSKDIGSESTEDQAMEDIK |
| | (B. Taurus) aa 25-199 | QMEAESISSSEEIVPNSVEQKHIQKEDVPSERYLGYL |
| | with Methionine | EQLLRLKKYKVPQLEIVPNSAEERLHSMKEGIHAQ |
| | | QKEPMIGVNQELAYFYPELFRQFYQLDAYPSGAWY |
| | | YVPLGTQYTDAPSFSDIPNPIGSENSGKTTMPLW |
| 70 | Bovine Alpha-S1 Casein | APFPEVFGKEKVNELSKDIGSESTEDQAMEDIKQME |
| | (B. Taurus) aa 26-199 | AESISSSEEIVPNSVEQKHIQKEDVPSERYLGYLEQL |
| | | LRLKKYKVPQLEIVPNSAEERLHSMKEGIHAQQKEP |
| | | MIGVNQELAYFYPELFRQFYQLDAYPSGAWYYVPL |
| | | GTQYTDAPSFSDIPNPIGSENSGKTTMPLW |
| 71 | Bovine Alpha-S1 Casein | MAPFPEVFGKEKVNELSKDIGSESTEDQAMEDIKQ |
| | (B. Taurus) aa 26-199 | MEAESISSSEEIVPNSVEQKHIQKEDVPSERYLGYLE |
| | with Methionine | QLLRLKKYKVPQLEIVPNSAEERLHSMKEGIHAQQ |
| | | KEPMIGVNQELAYFYPELFRQFYQLDAYPSGAWYY |
| | | VPLGTQYTDAPSFSDIPNPIGSENSGKTTMPLW |
| 72 | Bovine Alpha-S1 Casein | MEAESISSSEEIVPNSVEQKHIQKEDVPSERYLGYLE |
| | (B. Taurus) aa 60-199 | QLLRLKKYKVPQLEIVPNSAEERLHSMKEGIHAQQ |
| | | KEPMIGVNQELAYFYPELFRQFYQLDAYPSGAWYY |
| | | VPLGTQYTDAPSFSDIPNPIGSENSEKTTMPLW |

### EXAMPLES

The following illustrative examples are representative of embodiments of the compositions and methods described herein and are not meant to be limiting in any way.

### Example 1. Intra-Micellar Crosslinking Using Transglutaminase (TG) with Alpha and Kappa Caseins, Curd, and Cheesemaking

Micelles containing hypophosphorylated alpha casein and kappa casein were formed as follows: 11.9 mg/ml of hypophosphorylated alpha casein (Sigma Aldrich) was mixed with 2.1 mg/ml of kappa casein (Sigma Aldrich), and micelles were induced using calcium, phosphate, and citrate salts at room temperature. The calcium, phosphate, and citrate salts were at the following final concentrations: calcium 18.5 mM, phosphate 12.4 mM, and citrate 6.15 mM.

Transglutaminase (TG, Activa TI) was added at 0.25% to the alpha casein and kappa casein mixes at one of four stages in the micelle induction process: 1) to the alpha casein by itself, prior to any kappa casein addition or salts induction of micelles, 2) immediately after mixing alpha casein and kappa casein, prior to any salts induction of micelles, 3) after mixing alpha casein and kappa casein and incubating the mixture overnight at room temperature, prior to any salts induction of micelles, or 4) after mixing alpha casein and kappa casein and after inducing casein micelles via addition of salts. The only variable in the experiment was the point in the protocol in which transglutaminase treatment of proteins was applied. The transglutaminase incubation step was performed at 40° C. for 1 hour, followed by 78° C. inactivation for 10 minutes. Control experiments were done, lacking transglutaminase, which included the same incubation and inactivation step (40° C. for 1 hour, followed by 78° C. inactivation for 10 minutes), to control for the effect of the temperature changes themselves.

The resulting colloids were evaluated using dynamic light scattering (DLS) for particle size measurement. Samples were diluted to a concentration of 1.4 mg/mL of protein or less in filtered (220 nm) milliQ water. 50 μL samples were used for measurement and three replicates were measured at a 1730 detection angle over the amount of time determined by the instrument using Zetasizer (Malvern). The data was analyzed using the Zetasizer's small peak analysis mode. Colloids formed were further evaluated using native PAGE (4-20% Tris-Glycine gel, 55V for 3 hours) for estimating the depletion of monomeric caseins.

Colloids formed were subjected to curd and cheesemaking, and the efficiency of cheesemaking was estimated (curd formation quality, stretch quality, and yield). Samples were acidified by titrating 6.6 citric acid until the pH was from 5.0 to 5.2. Samples were then renneted at room temperature using 0.15 rennet solution at 1.36 of the final micellar colloid volume. Curds were spun at 1,000×g for 1 minute, drained from liquid, and submerged in a 65° C. water bath (duration dependent on curd size), stretched into pasta filata-like balls (mozzarella balls), placed on a wipe to remove excess moisture, and weighed. Control sample colloids were prepared at 14 mg/ml casein concentration dilution and measured in triplicates in an independent experiment.

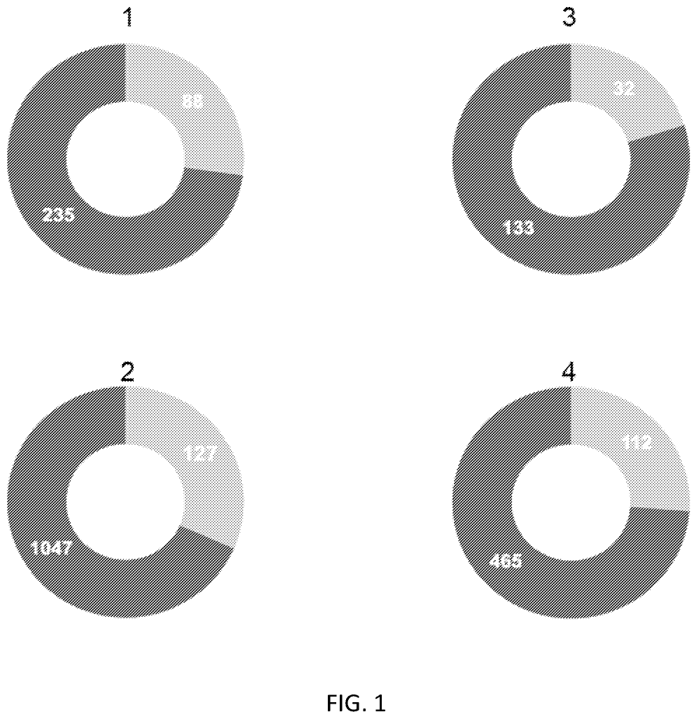

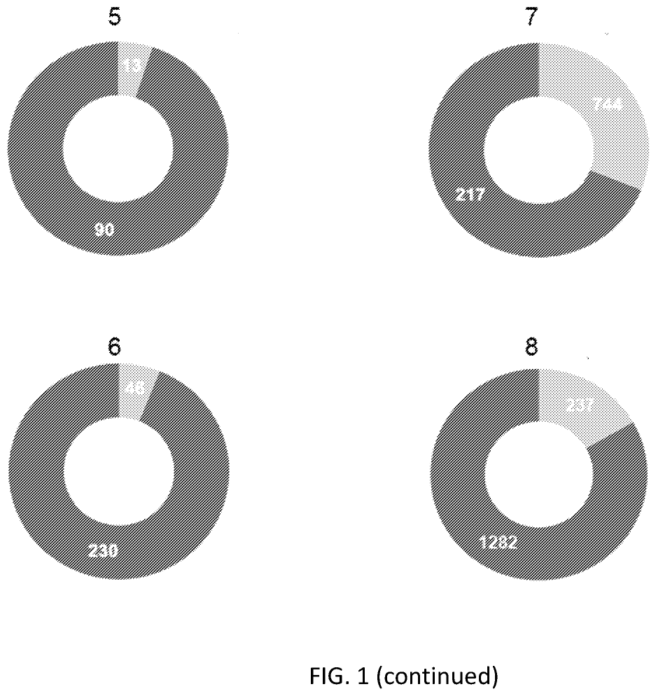

FIG. 1 depicts average micellar diameter of casein micelles. Adding TG after the micelles were induced provided the greatest improvement over a no TG control. More casein was micellar and trapped in wells on PAGE, and nearly all monomeric casein was depleted. FIG. 1 demonstrates that while incubation at 40° C. of hypophosphorylated alpha casein and kappa casein salts-induced micelles leads to their aggregation, incubation in presence of TG led to micellar particle stabilization. Table 2 depicts figure legends for FIG. 1.

TABLE 2
Figure legends for FIG. 1.

| x | Hyphophosphorylated alpha |
|---|---|
| y | Hyphophosphorylated alpha + kappa |
| 1 | Hypophosphorylated alpha + TG + kappa + salts |
| 2 | Hypophosphorylated alpha + kappa + salts |
| 3 | Hypophosphorylated alpha + kappa + TG + salts |
| 4 | Hypophosphorylated alpha + kappa + TG + salts |
| 5 | Hypophosphorylated alpha + kappa + O/N incubation + salts +TG |
| 6 | Hypophosphorylated alpha + kappa + O/N incubation + salts |
| 7 | Hypophosphorylated alpha + kappa + salts + TG |
| 8 | Hypophosphorylated alpha + kappa + salts |

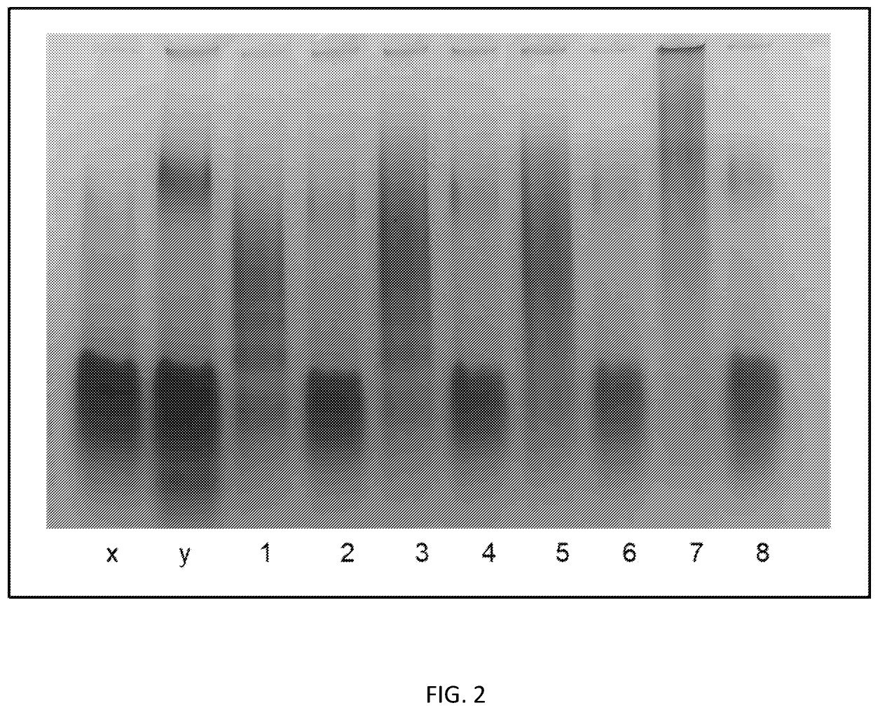

FIG. 2 demonstrates that TG treatment of mixtures prior to micelle induction (salt addition) led to primarily multimerization of hypophosphorylated alpha casein rather than micellar stabilization. Table 3 depicts figure legends for FIG. 2.

TABLE 3
Figure legends for FIG. 2.

| x | Hyphophosphorylated alpha |
|---|---|
| y | Hyphophosphorylated alpha + kappa |
| 1 | Hypophosphorylated alpha + TG + kappa + salts |
| 2 | Hypophosphorylated alpha + kappa + salts |
| 3 | Hypophosphorylated alpha + kappa + TG + salts |
| 4 | Hypophosphorylated alpha + kappa + TG + salts |
| 5 | Hypophosphorylated alpha + kappa + O/N incubation + salts +TG |
| 6 | Hypophosphorylated alpha + kappa + O/N incubation + salts |
| 7 | Hypophosphorylated alpha + kappa + salts + TG |
| 8 | Hypophosphorylated alpha + kappa + salts |

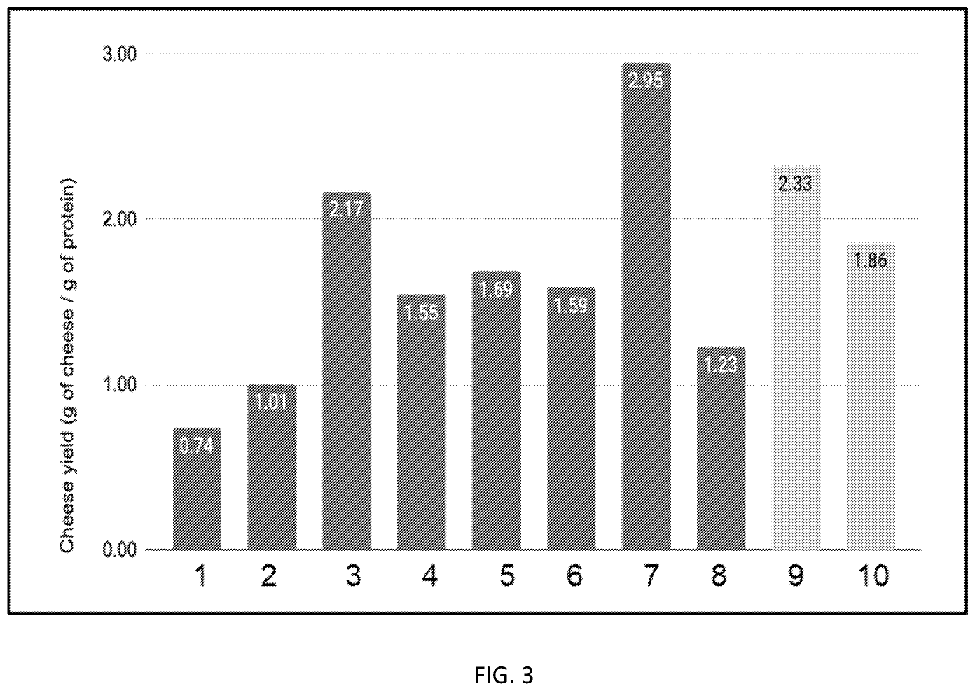

Cheese yields were as follows: 2.33 for skim milk (+/−0.18 stdv on triplicate) and 1.86 for micellar casein (+/−0.30 stdv on triplicate). Cheese yields for pasta filata-like balls made from the crosslinked alpha casein and kappa casein colloids are shown in FIG. 3, with the skim milk and micellar casein controls on the far-right of the bar graph. Table 4 depicts figure legends for FIG. 3.

TABLE 4
Figure legends for FIG. 3.

| | |
|---|---|
| 1 | Hypophosphorylated alpha + TG + kappa + salts |
| 2 | Hypophosphorylated alpha + kappa + salts |
| 3 | Hypophosphorylated alpha + kappa + TG + salts |
| 4 | Hypophosphorylated alpha + kappa + TG + salts |
| 5 | Hypophosphorylated alpha + kappa + O/N incubation + salts +TG |
| 6 | Hypophosphorylated alpha + kappa + O/N incubation + salts |
| 7 | Hypophosphorylated alpha + kappa + salts + TG |
| 8 | Hypophosphorylated alpha + kappa + salts |
| 9 | Skim milk |
| 10 | Micellar casein |

### Example 2. Comparison of Micelle Formation Made from Native and Reduced (Non-Native) Post-Translational Modification of Caseins Using Intra-Micellar Crosslinking

Hypophosphorylated alpha casein (Sigma Aldrich), native alpha casein (Sigma Aldrich), or a mixture of alpha casein and beta casein (Sigma Aldrich) were used with kappa casein (Sigma Aldrich) to form intra-micellar crosslinked casein micelles. 11.9 mg/ml of alpha casein alone, or 7 mg/ml of alpha casein with 4.9 mg/ml beta casein, was mixed with 2.1 mg/ml of kappa casein, and micelles were induced using calcium, phosphate, and citrate salts at room temperature. The calcium, phosphate, and citrate salts were at the following final concentrations: calcium 18.5 mM, phosphate 12.4 mM, and citrate 6.15 mM.

Transglutaminase (TG, Activa TI) was added at 0.25% to the salts-induced casein micelles. The transglutaminase incubation step was performed at 40° C. for 1 hour, followed by 78° C. inactivation for 10 minutes. Control experiments were performed lacking transglutaminase, and included the same incubation and inactivation step (40° C. for 1 hour, followed by 78° C. inactivation for 10 minutes), to control for the effect of the temperature changes themselves.

The resulting colloids were evaluated using dynamic light scattering (DLS) for particle size measurement. Samples were diluted to a concentration of 1.4 mg/mL of protein or less in filtered (220 nm) milliQ water. 50 μL samples were used for measurement and three replicates were measured at a 173° detection angle over the amount of time determined by the instrument using Zetasizer (Malvern). The data was analyzed using the Zetasizer's small peak analysis mode.

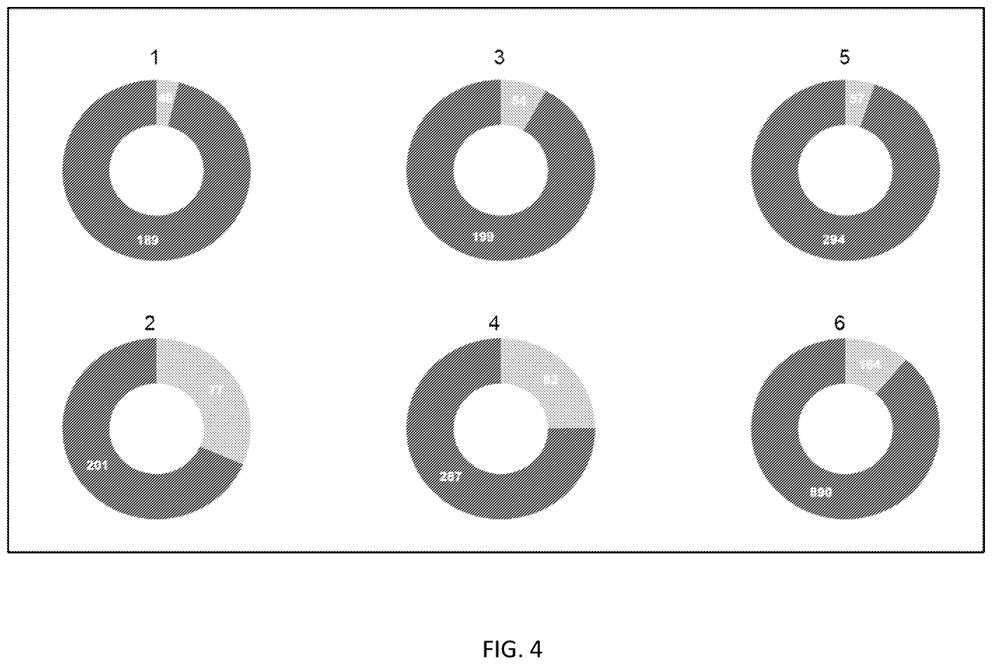

A majority of casein protein was not in micellar form, as is indicated in FIG. 4 for at least the crosslinked samples. DLS was used to detect particles. The majority of particles detected in all three mixtures were sized at average particle size from 200 nm to 300 nm, with or without TG. Table 5 depicts figure legends for FIG. 4.

TABLE 5
Figure legends for FIG. 4.

| | |
|---|---|
| 1 | Alpha + beta + kappa + TG |
| 2 | Alpha + beta + kappa |
| 3 | Alpha + kappa + TG |
| 4 | Alpha + kappa |
| 5 | Hypophosphorylated alpha + kappa + TG |
| 6 | Hypophosphorylated alpha + kappa |

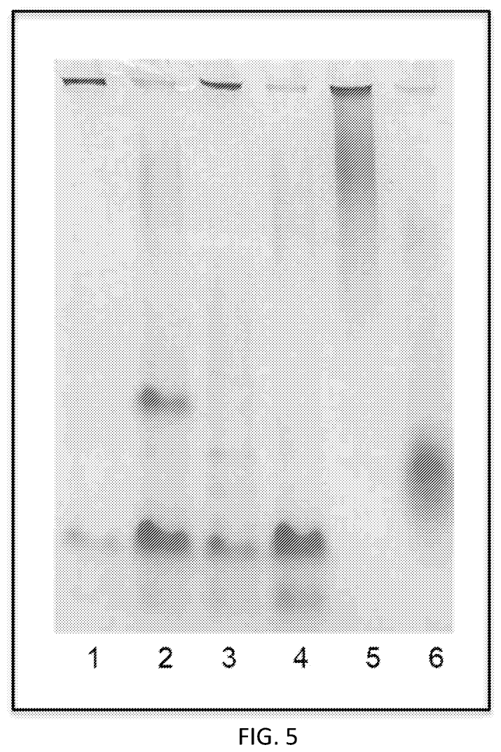

FIG. 5 indicates that only for the hypophosphorylated alpha casein sample was a majority of the protein in a micellar fraction that is crosslinked. Table 6 depicts figure legends for FIG. 5.

TABLE 6
Figure legends for FIG. 5.

| | |
|---|---|
| 1 | Alpha + beta + kappa + TG |
| 2 | Alpha + beta + kappa |
| 3 | Alpha + kappa + TG |
| 4 | Alpha + kappa |
| 5 | Hypophosphorylated alpha + kappa + TG |
| 6 | Hypophosphorylated alpha + kappa |

In controls where the hypophosphorylated alpha casein with kappa casein was not exposed to TG, the mixtures produced larger aggregates after the higher temperature treatment. Micelles formed with hypophosphorylated alpha casein and kappa casein upon TG treatment are slightly larger, about 300 nm in diameter, than micelles formed with native caseins (with or without beta casein), which are about 200 nm in diameter.

Colloids formed were further evaluated using native PAGE (4-20% Tris-Glycine gel, 55V for 3 hours) for estimating the depletion of monomeric caseins. FIG. 5 shows the results of the PAGE analysis. The majority of casein in crosslinked micelles with reduced phosphorylation (hypophosphorylated alpha casein) was found in micellar structures, which remained at the top of the well and do not enter the gel, with almost no monomeric casein appearing on the gel. This near-complete protein crosslinking achieved for micelles that contain the hypophosphorylated alpha casein was not observed for micelles formed from native caseins that have full phosphorylation. On the contrary, a significant amount of monomeric casein forms compared to treatments lacking TG is seen in the gel, and particularly for alpha casein. Given that the micelles formed with hypophosphorylated alpha casein and kappa casein are looser than micelles formed with native caseins (per the DLS data), the data indicates that TG reaches into the micelle core and crosslinks on intra-micellar positions with higher efficiency, rather than crosslinking on the outside of the micelle (on kappa casein itself) or across different micelles (inter-micellar).

### Example 3. Properties of Cheese Made from Native and Reduced (Non-Native) Post-Translational Modification of Caseins Using Intra-Micellar Crosslinking, Measured by Texture Analysis, and Tasting Experiment

Hypophosphorylated alpha casein (Sigma Aldrich) or native alpha casein (Sigma Aldrich) were mixed with kappa casein (Sigma Aldrich) to form intra-micellar crosslinked casein micelles, using 27.2 mg/ml of the alpha casein and 4.8 mg/ml of the kappa casein. Micelles were induced using calcium, phosphate, and citrate salts at room temperature. The calcium, phosphate, and citrate salts were at the following final concentrations: calcium 30.75 mM, phosphate 16.5 mM, citrate 8.2 mM.

Transglutaminase (TG, Activa TI) was added at 0.25% to such salts-induced casein micelles, made from the hypophosphorylated alpha casein. The transglutaminase incubation step was performed at 40° C. for 15 minutes or 30 minutes, followed by 78° C. inactivation for 10 minutes. A control experiment, lacking transglutaminase, was performed on a “native” dairy system of alpha casein and kappa casein.

Samples were acidified by titrating 6.6% citric acid until the pH was from 5.0 to 5.2. Samples were then renneted at room temperature using 0.15% rennet solution at 1.36% of the final micellar colloid volume. Curds were spun at 1,000×g for 1 minute, drained from liquid, and submerged in a 65° C. water bath (duration dependent on curd size), stretched into mozzarella balls, placed on a wipe to remove excess moisture, and weighed. Each cheese sample was made in triplicates and it was subjected to texture analysis.

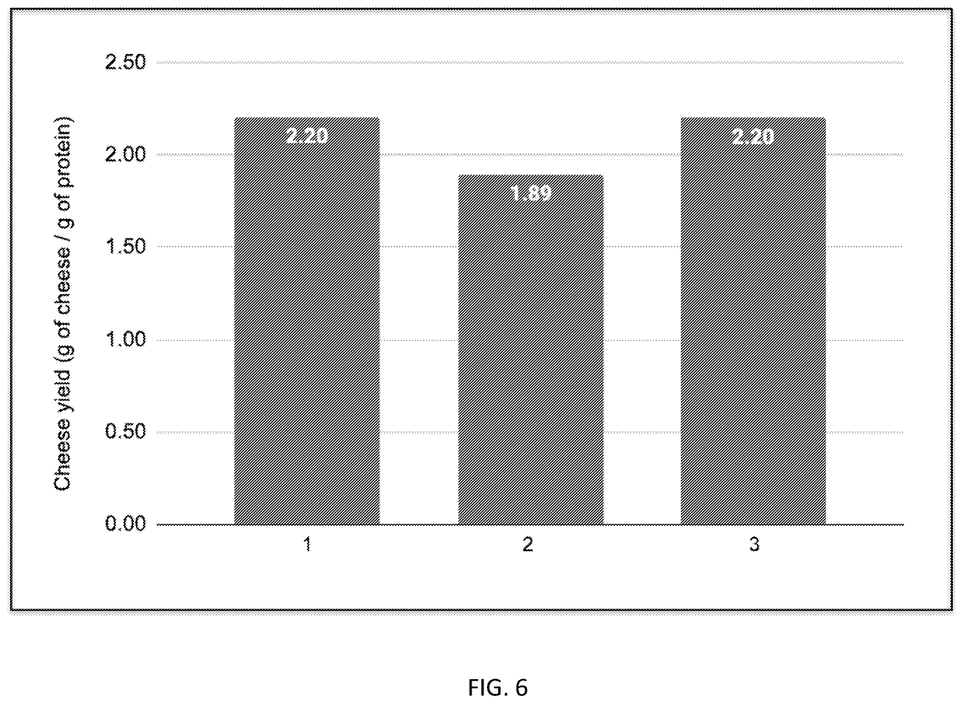

FIG. 6 shows the cheese yields from the alpha casein and kappa casein micelles. The crosslinked micelles containing the hypophosphorylated alpha casein were comparable to cheese yields of micelles that have native alpha casein phosphorylation. Table 7 depicts figure legends for FIG. 6.

TABLE 7
Figure legends for FIG. 6.

| | |
|---|---|
| 1 | Alpha + kappa |
| 2 | Alpha + kappa + TG (15 min incubation) |
| 3 | Alpha + kappa + TG (30 min incubation) |

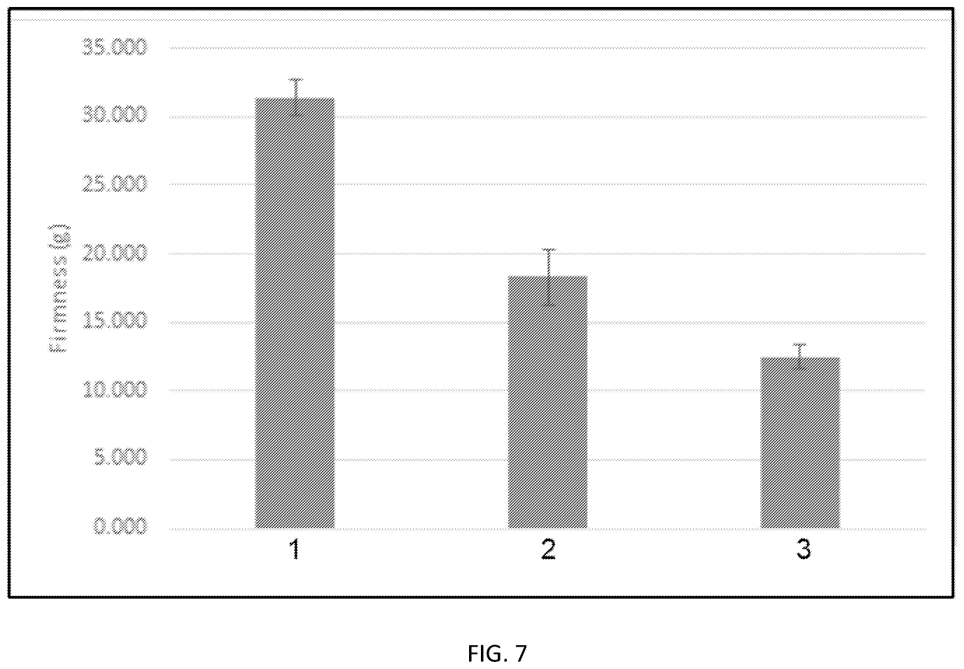

Additionally, and surprisingly, the texture and softness of pasta-filata (mozzarella) cheese improves significantly, as seen in FIG. 7. Table 8 depicts figure legends for FIG. 7.

TABLE 8
Figure legends for FIG. 7.

| | |
|---|---|
| 1 | Alpha + kappa |
| 2 | Alpha + kappa + TG (15 min incubation) |
| 3 | Alpha + kappa + TG (30 min incubation) |

A pasta-filata cheese made from milk using this process typically falls in the range of 10-15 g in firmness in this same texture analysis test (data not shown). In an internal tasting event, pasta-filata cheese samples made from crosslinked micelles containing the hypophosphorylated alpha casein were described as milk-like tasting and milk-like soft cheeses, and they were unanimously agreed as preferred compared to the control experiment (cheese from micelles that have native alpha casein phosphorylation).

### Example 4. Micelle Formation, Curd, and Cheesemaking Made from Recombinant Caseins Lacking Post-Translational Modifications (PTMs) Using Intra-Micellar Crosslinking

Native bovine alpha casein (in-house purified from cow's milk), hypophosphorylated bovine alpha casein (Sigma Aldrich) and recombinantly-produced bovine alpha-S1 casein (lacking PTMs), were used with bovine kappa casein purified from cow's milk (in-house purified from cow's milk) and recombinantly-produced sheep kappa casein to form intra-micellar crosslinked casein micelles. 10.4 mg/ml of alpha casein was mixed with 3.6 mg/ml kappa casein, and micelles were induced at room temperature by adding 18.5 mM calcium, 12.4 mM phosphate, and 6.15 mM citrate. Micelles containing recombinantly produced proteins were induced by adding 27 mM calcium, 20 mM phosphate, and 10 mM citrate. Transglutaminase (TG, Activa TI) was added at 0.125% to the salts-induced casein micelles and incubated at 40° C. for 30 minutes. In control samples, water was added instead of TG solution.

For particle sizing measurement, samples were diluted to a 1.4 mg/mL concentration of protein or less in filtered (220 nm) milliQ water. 50 μl samples were used for measurement, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and 6.4. Samples were then renneted at room temperature using 0.15% rennet solution at 1.36% of the final micellar colloid volume. Curds were spun at 1,000×g for 1 minute, drained from liquid and submerged in a 70° C. water bath (duration dependent on curd size), stretched into mozzarella balls, placed on a wipe to remove excess moisture, and weighed.

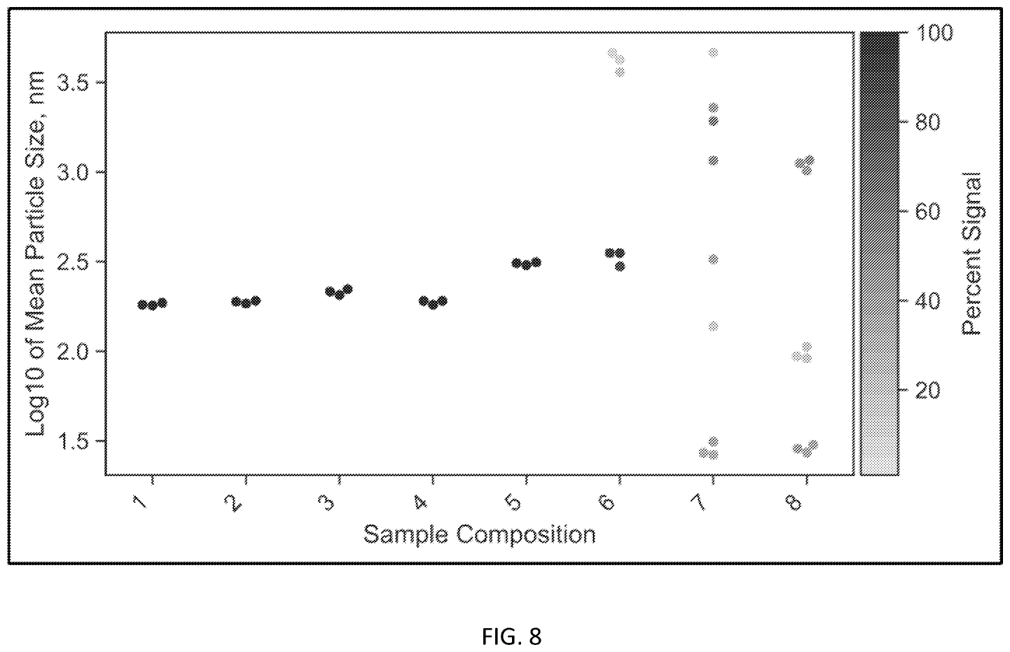

FIG. 8 shows micelle size did not vary significantly after the TG treatment. Recombinant bovine alpha-S1 casein formed somewhat larger micelles (260-360 nm) compared to native (180-200 nm) or hypophosphorylated (180-230 nm) alpha casein when combined with native bovine kappa casein. FIG. 8 also shows recombinant bovine alpha casein and recombinant sheep kappa casein colloids have a broad particle size range where the main particle population is in the range of 1000-2000 nm in size and there are submicelles at 20-40 nm present. As micelles in the range of 500-900 nm are formed upon combining recombinant bovine alpha casein and sheep kappa casein with no larger aggregates present, it is likely that the high-temperature incubation performed both for TG treated and untreated sample causes micelles to aggregate into larger particles. Table 9 depicts figure legends for FIG. 8.

TABLE 9
Figure legends for FIG. 8.

| | |
|---|---|
| 1 | Alpha + kappa + TG |
| 2 | Alpha + kappa |
| 3 | Hypophosphorylated alpha + kappa + TG |
| 4 | Hypophosphorylated alpha + kappa |
| 5 | Recombinant alpha-S1 + kappa + TG |
| 6 | Recombinant alpha-S1 + kappa |
| 7 | Recombinant alpha-S1 + recombinant sheep kappa + TG |
| 8 | Recombinant alpha-S1 + recombinant sheep kappa |

Untreated micelles made from hypophosphorylated bovine alpha casein and native bovine kappa casein did not form stable curds, whereas upon TG treatment, micelles form strong, cohesive, and stable curds. All other samples, TG treated and untreated, form stable, and cohesive curds.

TABLE 10
Curd formation, mozzarella cheese yield (g of cheese/g of protein),
stretch and melt extent of cheeses made from colloids in FIG. 8.

| | Avg Cheese | | Avg Cheese | | Avg Cheese | | % Cheese |
|---|---|---|---|---|---|---|---|
| | Stretch | | melt | | yield | | yield |
| | TG | No TG | TG | No TG | TG | No TG | increase |
| Native bovine alpha + native | ++ | ++++ | ++ | ++++ | 1.9 | 1.5 | 40% |
| bovine kappa | | | | | | | |
| Hypophosphorylated bovine | ++ | ++++ | ++ | ++++ | 1.45 | 1.05 | 40% |
| alpha + native bovine kappa | | | | | | | |
| Recombinant bovine alpha + | +++ | ++++ | ++ | ++ | 1.85 | 1.60 | 25% |
| native bovine kappa | | | | | | | |
| Recombinant bovine alpha + | +++++ | +++++ | +++++ | +++++ | 1.75 | 1.45 | 30% |
| recombinant sheep kappa | | | | | | | |

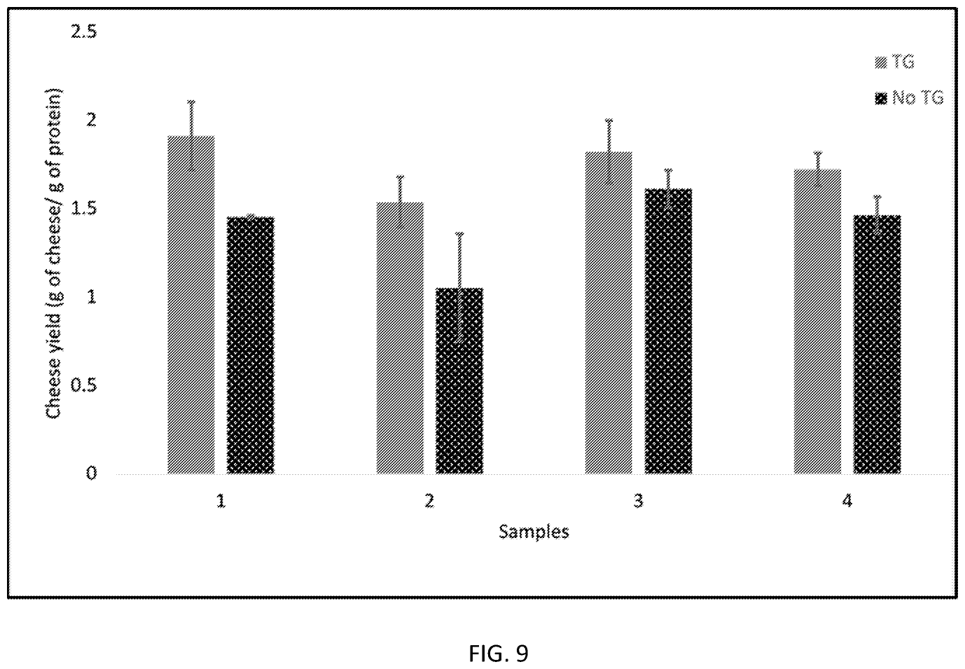

FIG. 9 demonstrates that cheese yields are higher in all the TG treated samples compared to untreated samples. Table 10 demonstrates that TG treated and untreated micelles made from recombinant bovine alpha casein combined with recombinant kappa casein, made the best cheese that stretched and melted even better than the cheeses made using native bovine caseins. The amount of stretch or melt is indicated by plus signs (+), with “+” being the poorest stretch or melt, and “+++++” being the best stretch or melt. TG treatment slightly reduced the stretch and melt of cheese made from native or hypophosphorylated bovine alpha casein combined with native bovine kappa casein. However, TG treatment did not affect the properties of cheese made from combining recombinant bovine alpha casein and recombinant sheep kappa casein. Table 11 depicts figure legends for FIG. 9.

TABLE 11
Figure legends for FIG. 9.

| | |
|---|---|
| 1 | Alpha + kappa + TG |
| 2 | Alpha + kappa |
| 3 | Hypophosphorylated alpha + kappa + TG |
| 4 | Hypophosphorylated alpha + kappa |
| 5 | Recombinant alpha-S1 + kappa + TG |
| 6 | Recombinant alpha-S1 + kappa |
| 7 | Recombinant alpha-S1 + recombinant sheep kappa + TG |
| 8 | Recombinant alpha-S1 + recombinant sheep kappa |

### Example 5. Single Casein Analysis

To assess micelle and cheese formation with single caseins, alpha casein (Sigma Aldrich), beta casein (Sigma Aldrich), and kappa casein (Sigma Aldrich) were each used singly in experiments in an attempt to form single-casein micelles and evaluate the effect of crosslinking with transglutaminase. 14 mg/ml of a single casein was used in water, and the sample was treated with micelle-inducing salt conditions, using calcium, phosphate, and citrate salts at room temperature. The calcium, phosphate, and citrate salts were at the following final concentrations: calcium 18.5 mM, phosphate 12.4 mM, and citrate 6.15 mM.

Transglutaminase (TG, Activa TI) was added at 0.25% to such salts-treated samples. The transglutaminase incubation step was performed at 40° C. for 1 hour, followed by 78° C. inactivation for 10 minutes. Control experiments, lacking transglutaminase, included the same incubation and inactivation step (40° C. for 1 h, followed by 78° C. inactivation for 10 minutes), to control for the effect of the temperature changes themselves.

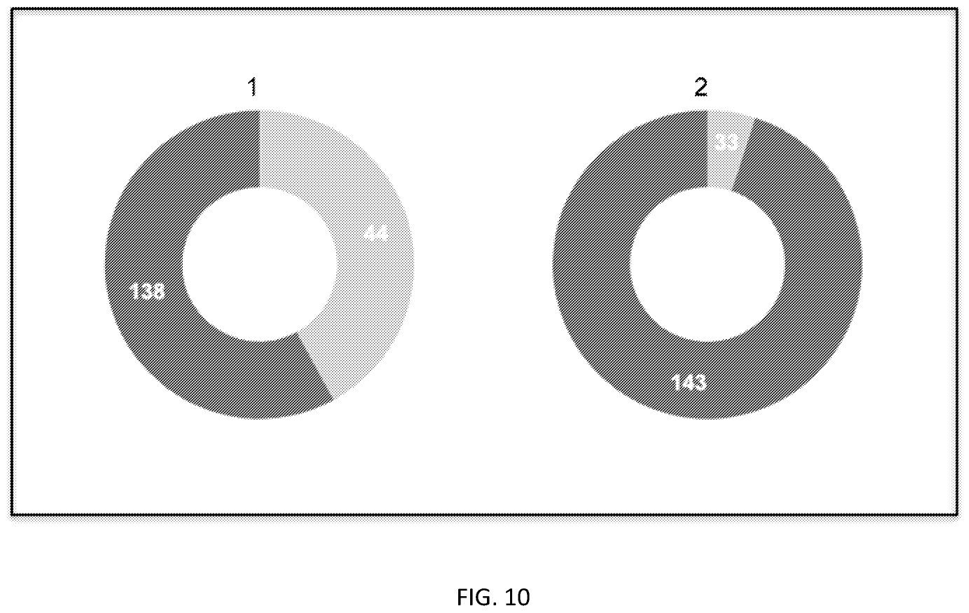

The resulting colloids were evaluated using dynamic light scattering (DLS) for particle size measurement. Samples were diluted to a concentration of 1.4 mg/mL of protein or less in filtered (220 nm) milliQ water. 50 μL samples were used for measurement and three replicates were measured at a 1730 detection angle over the amount of time determined by the instrument using Zetasizer (Malvern). The data was analyzed using the Zetasizer's small peak analysis mode. DLS analysis is shown in FIG. 10. DLS measurements returned no signal for alpha casein and beta casein samples due to too-low turbidity, with or without TG added (meaning no particles or aggregates were detected). However, kappa casein samples showed particles sized at about 140 nm, with and without TG added (FIG. 10). All samples showed visually some amount of protein precipitates, which sedimented very quickly and were not detected in the light scattering (DLS) analysis. Table 12 depicts figure legends for FIG. 10.

TABLE 12
Figure legends for FIG. 10.

| | |
|---|---|
| 1 | Kappa + TG |
| 2 | Kappa |

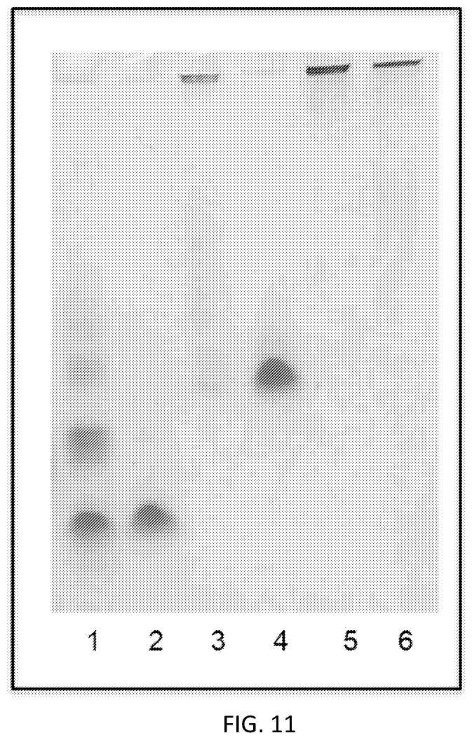

Colloids formed were further evaluated using native PAGE (4-20% Tris-Glycine gel, 55V for 3 hours) for estimating the depletion of monomeric caseins. Results are shown in FIG. 11. Alpha casein incubated with TG displayed multimerization (laddering on the gel) but no larger particles were observed in the well that were too big to enter the gel and migrate, suggesting that micelles were not formed from alpha casein alone. Beta casein also showed multimerization (laddering on the gel) and some number of larger particles formed that did not enter the gel. Interestingly, the kappa casein samples contained particles that did not dissociate under the native PAGE conditions; this occurred for kappa casein incubated with and without TG. However, the amount of protein detected in the well was higher for the kappa casein with TG incubation, whereas some monomeric protein was still detected in the sample not exposed to TG. Table 13 depicts figure legends for FIG. 11.

TABLE 13
Figure legends for FIG. 11.

| | |
|---|---|
| 1 | Alpha + TG |
| 2 | Alpha |
| 3 | Beta + TG |
| 4 | Beta |
| 5 | Kappa + TG |
| 6 | Kappa |

The samples were also subjected to curd and cheesemaking, and if such a formation occurred, the efficiency of cheesemaking was estimated (curd formation quality, stretch quality, and yield). Samples were acidified by titrating 6.6% citric acid until the pH was from 5.0 to 5.2. Samples were then renneted at room temperature using 0.15% rennet solution at 1.36% of the final micellar colloid volume. Curds were spun at 1,000×g for 1 minute, drained from liquid and submerged in a 65° C. water bath (duration dependent on curd size), stretched into mozzarella balls, placed on a wipe to remove excess moisture, and weighed.

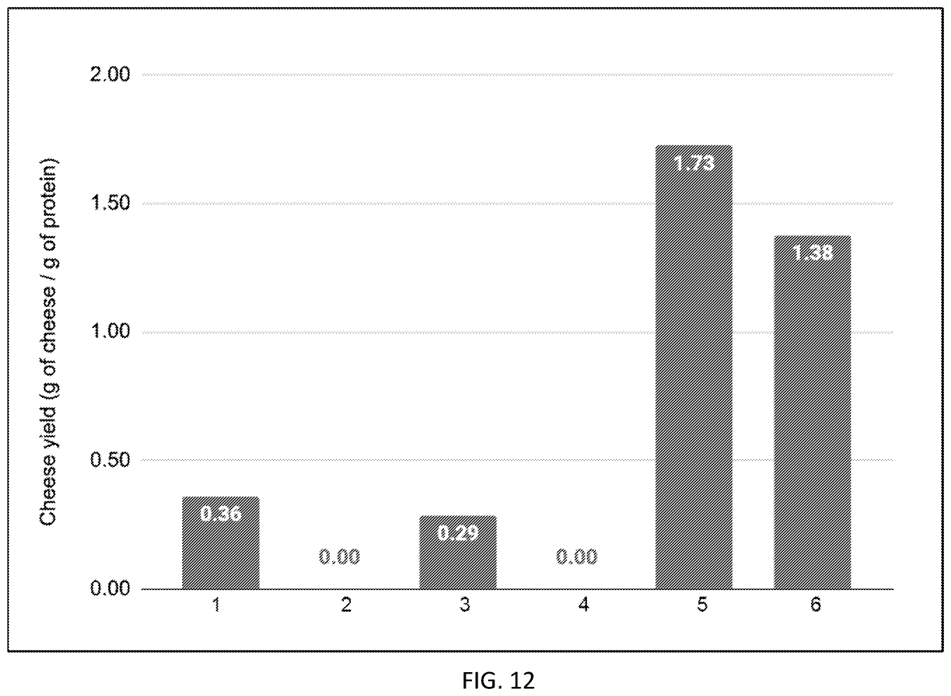

FIG. 12 shows the results from the cheesemaking. No true curd was formed for any of the samples. Instead protein precipitates and/or chunks were formed. For alpha casein and beta casein, the precipitates were crumbly and were not able to align under hot water treatment and be stretched (both samples with TG incubation and without TG). No cheese was made from those samples, but still a crashed-out crumble weight was reported (FIG. 12). Kappa casein by itself, on the contrary, showed great pasta-filata like properties, with or without TG treatment. It showed typical pasta-filata like stretch in hot water treatment and gave cheese-like products of decent yields (FIG. 12). Treatment with TG further improved yield of kappa casein cheese by 25%.

FIG. 12 shows cheese yields for pasta filata-like balls made from kappa casein (k) colloids with and without transglutaminase (TG) treatment. Alpha casein (a) and beta casein (b) colloids did not produce actual pasta filata-like cheese, however, the total yield of the protein crash out and/or precipitate is reported as yield. Table 14 depicts figure legends for FIG. 12.

TABLE 14
Figure legends for FIG. 12.

| | |
|---|---|
| 1 | Alpha + TG |
| 2 | Alpha |
| 3 | Beta + TG |
| 4 | Beta |
| 5 | Kappa + TG |
| 6 | Kappa |

### Example 6. Micelle-Like Particles Made from Single Casein, their Colloid Properties, and Cheesemaking

Native bovine kappa casein purified from cow's milk (In-house Purified) was used to form single-casein micelle-like particles, with and without the transglutaminase (TG) treatment, to evaluate the effect of TG induced crosslinking. 14 mg/ml of single casein was used in water, and micelles were induced using 12.4 mM phosphate, 6.15 mM citrate, and 18.5 mM calcium. Transglutaminase (Activa TI) was added at 0.5% to such salt-treated samples and incubated at 40° C. for 30 minutes. To keep the process consistent, control samples lacking TG included the same incubation step (40° C. for 30 minutes).

The resulting colloids were evaluated using dynamic light scattering (DLS) for particle size measurement. Samples were diluted to a concentration of 1.4 mg/mL of protein or less in filtered (220 nm) milliQ water. 50 μl samples were used for particle size measurements, and three replicates were measured at a 173° detection angle over the amount of Zetasizer's small peak analysis mode. For turbidity measurements, samples were diluted to a concentration of 0.7 mg/ml in filtered (220 nm) milliQ water, and absorbance was measured at 450 nm in 1 ml cuvettes using Spectramax.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and 5.4. Samples were then renneted at room temperature using 0.15% rennet solution at 1.36% of the final micellar colloid volume. Curd quality was judged by performing a tube inversion test. Here, the tubes having the curd are kept upside down. If the curd or whey doesn't slide down the tube, then it is considered as strong, full, and stable curd. Curds were spun at 1,000×g for 1 minute, drained from liquid and submerged in a 70° C. water bath (duration dependent on curd size), stretched into mozzarella balls, placed on a wipe to remove excess moisture, and weighed.

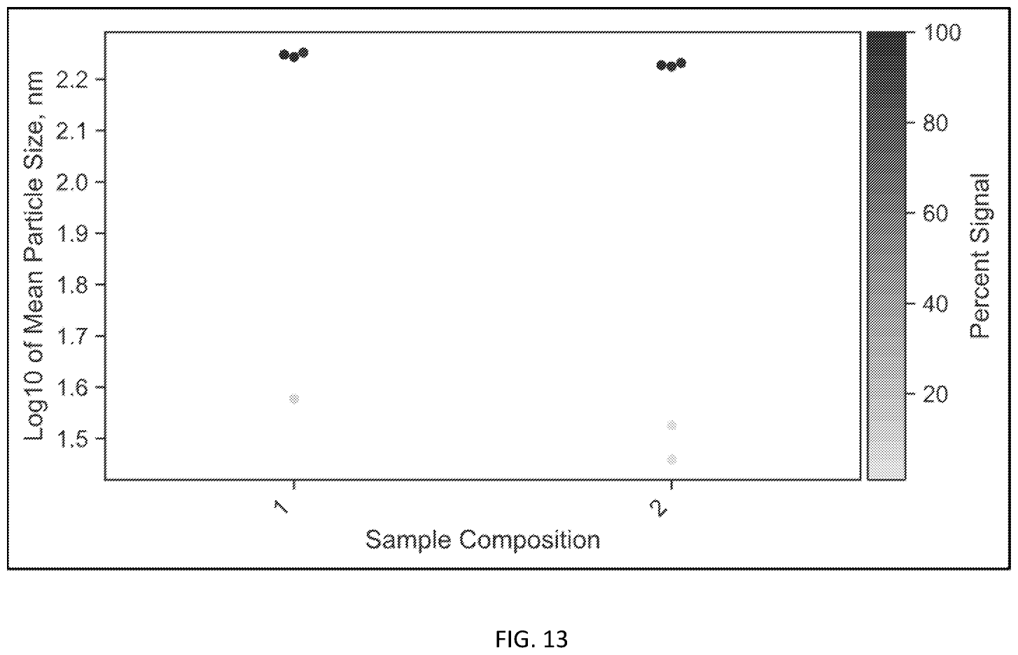

FIG. 13 shows salt-induced kappa casein can form micelle-like particles in the size range of 160-170 nm, and TG treatment did not change micelle size. During acidification, the untreated kappa casein colloids precipitated to some extent, whereas the TG-treated kappa casein colloids did not precipitate at all suggesting further micelle stabilization at that particular pH after cross-linking. Table 15 depicts figure legends for FIG. 13.

TABLE 15
Figure legends for FIG. 13.

| | |
|---|---|
| 1 | Kappa + TG |
| 2 | Kappa |

TABLE 16
Curd formation, mozzarella cheese yield (g of cheese/g of protein),
stretch and melt extent of cheeses made from colloids in FIG. 13.

| | Dairy K | Dairy K | % Increase in |
|---|---|---|---|
| | No TG | TG | cheese yield |
| Curd | Yes | Yes | 85 |
| Stretch | +++ | + | |
| Melt | ++ | + | |
| Cheese Yield (g | 1.55 | 2.40 | |
| cheese/g protein) | | | |

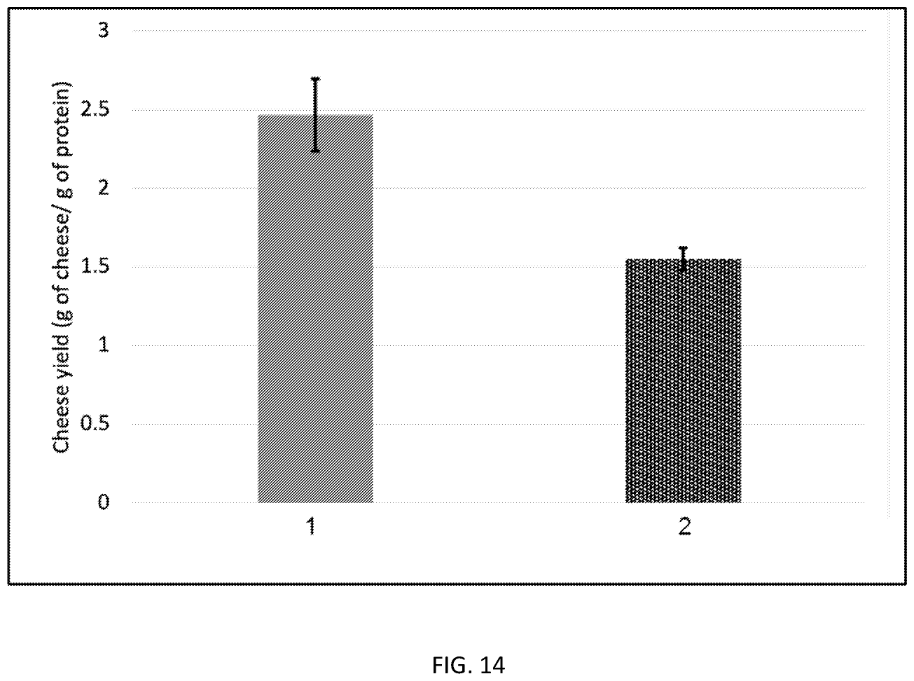

Table 16 shows that salt induced kappa casein colloids, with and without TG, formed strong, full, and stable curds that survived the tube inversion test. The amount of stretch or melt is indicated by plus signs (+), with “+” being the poorest stretch or melt, and “+++++” being the best stretch or melt. The spun down curd made from TG treated native kappa casein colloids appeared bigger, fluffier, and held more moisture, whereas the curd made from untreated kappa casein colloids was smooth, firm, and compact. FIG. 14 and Table 16 show TG treated native kappa casein colloid yielded an average of ˜ 85% more cheese than their untreated native kappa. TG treated native kappa casein cheese made in this particular example did not stretch or melt as untreated kappa casein cheese suggesting excess cross-linking is likely affecting the cheese properties. Further optimization including TG concentration and incubation temperature can yield a significant increase in the cheese yield while still retaining the pasta filata-like cheese properties or melt and stretch. Table 17 depicts figure legends for FIG. 14.

TABLE 17
Figure legends for FIG. 14.

| | |
|---|---|
| 1 | Kappa + TG |
| 2 | Kappa |

### Example 7. Micelle-Like Particles Made from Single Casein Lacking PTMs, their Colloid Properties, and Cheesemaking

Recombinant sheep kappa casein lacking PTMs was used to form single casein micelle-like particles, with and without the transglutaminase (TG) treatment, and to evaluate the effect of TG induced crosslinking. As a control sample, native bovine kappa casein purified from cow's milk (In-house Purification) was used to induce single-casein micelles, with and without the transglutaminase treatment. 14 mg/ml of a single casein was used in water, and micelles were induced using 12.4 mM phosphate, 6.15 mM citrate, and 18.5 mM calcium. Transglutaminase (Activa TI) was added at 0.125% to such salt treated samples and incubated at 40° C. for 30 minutes. Control samples, lacking TG, included the same incubation step (40° C. for 30 minutes) to keep the process consistent.

The resulting colloids were subjected to curd and cheesemaking, and the efficiency of cheesemaking was estimated (curd formation quality, stretch and melt quality, and yield). Samples were acidified by titrating 6.6% citric acid until the pH was between 5 and 5.4. Samples were then renneted at room temperature using 0.15% rennet solution at 1.36% of the final micellar colloid volume. Curd quality was judged by performing a tube inversion test. Here, the tubes having the curd are kept upside down. If the curd or whey doesn't slide down the tube, then it is considered as strong, full, and a stable curd. Curds were spun at 1,000×g for 1 minute, drained from liquid and submerged in a 70° C. water bath (duration dependent on curd size), stretched into mozzarella balls, placed on a wipe to remove excess moisture, and weighed.

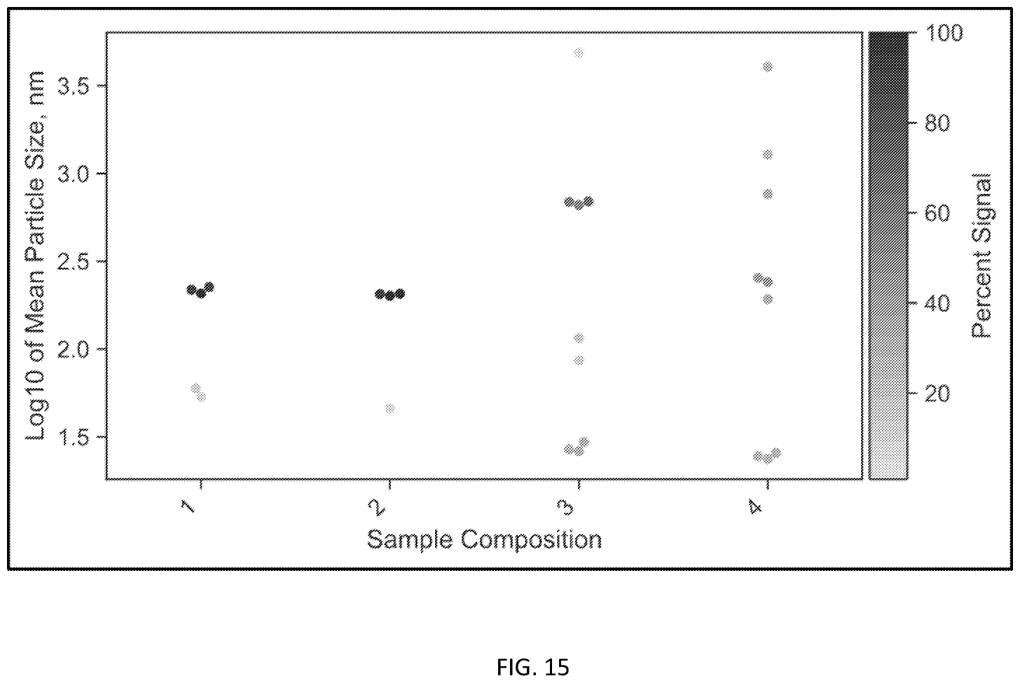

TABLE 18
Curd formation, mozzarella cheese yield (g of cheese/g of protein),
stretch and melt extent of cheeses made from colloids in FIG. 15.

| | Recombinant sheep kappa | | | |
|---|---|---|---|---|
| | casein | | Native bovine kappa casein | |
| | TG | No TG | TG | No TG |
| Curd | Yes | No | Yes | Yes |
| Stretch | +++++ | +++ | +++ | ++ |
| Melt | +++++ | +++ | +++ | ++ |
| Cheese yield | 1.65 | 1.50 | 1.50 | 1.45 |
| (g cheese/g | | | | |
| protein) | | | | |
| Increase in | 15 | N/A | 5 | N/A |
| cheese yield | | | | |
| (%) | | | | |

FIG. 15 shows that salt-induced native bovine kappa casein, with and without TG treatment, forms micelle-like particles that have a major proportion of particles in the size range of 170-240 nm. Salt induced untreated recombinant sheep kappa casein forms particles across the size range of 20 nm-4500 nm, with main particle population in the range of 160-320 nm, however with high variation in size among the replicates, indicating some instability of the particles. Interestingly, the TG treated salt-induced recombinant sheep kappa casein forms micelle-like particles having main particle population in narrow size range of 660-690 nm and a small proportion of sub-micelle-like particles in size range 20-30 nm, suggesting TG stabilizes the particles by cross-linking them. Table 19 depicts figure legends for FIG. 15.

TABLE 19
Figure legends for FIG. 15.

| | |
|---|---|
| 1 | Kappa + TG |
| 2 | Kappa |
| 3 | Recombinant sheep kappa + TG |
| 4 | Recombinant sheep kappa |

During acidification, untreated native bovine kappa casein and recombinant sheep kappa casein micelle-like particles showed low amount of crashed out particles. In contrast, the TG-treated samples did not show any crash outs, further suggesting micelle stabilization by TG-induced cross-linking.

Table 18 shows that TG treated recombinant sheep kappa casein colloids formed strong, full, and stable curds that passed the tube inversion test. However, the untreated sheep kappa casein colloid, which couldn't form stable micelles, resulted in forming loose and weak curd that didn't pass the tube inversion test. The cheese made from TG treated sheep kappa casein colloid melted and stretched significantly better than the cheese made from untreated sheep kappa casein colloid, and significantly better than any of the cheeses, treated or untreated, made from native bovine kappa casein colloids. The micelles are likely stabilized by cross-linking in the TG treated sheep kappa casein colloid; this translates to improved pasta filata-like properties of the resulting cheese, such as melt and stretch.

The untreated and TG treated native bovine kappa colloids formed curds that stretched and melted well. The overall properties of cheese made from TG treated recombinant sheep kappa colloids were even superior to native bovine kappa casein colloid (both TG treated and untreated), suggesting the favourable pasta filata-like property of cheese is likely an attribute of sheep kappa casein protein itself. Table 18 shows TG treatment led to a 15% increase in the recombinant sheep kappa casein cheese yield. At the given concentration, TG treatment did not increase the yield of cheese made from native bovine kappa casein. The optimal TG concentration to achieve ideal increase in yield and desirable melt and stretch likely varies for the type of casein protein used to form micelle-like particles and their colloids, and can be further optimized for.

### Example 8. Expression of Casein Proteins in Lactococcus lactis Via Nisin-Inducible System (NICE)

Constructs Design, Cloning, and Transformation

Bovine kappa casein (variant B) and bovine alpha-S1 casein (variant C) protein coding sequences (without the native signal peptide) were codon-optimized for expression in Lactococcus lactis and a synthetic operon was constructed for co-expression and secretion of the two proteins under a nisin-inducible promoter. Signal peptide sequence from natively secreting lactococcal protein Usp45 was used to drive protein secretion. A synthetic operon was then cloned into an E. coli custom vector via restriction digest compatible sites and confirmed via Sanger sequencing, from which it was subcloned into nisin-inducible pNZ8149 vector via restriction digestion and ligation. The vector was transformed into compatible L. lactis strain NZ3900 via electroporation and completely defined media (CDM) supplemented with lactose was used for selection. Positive clones were confirmed via colony PCR and three positive clones were taken forward for the protein expression induction and analysis.

Protein Expression and Analysis

Individual colonies were grown at 30° C. in liquid culture and protein production was induced with nisin for 2.5 hours (control samples left uninduced). Cells were then harvested by centrifugation and TCA-precipitated supernatants and lysed cell pellets were analysed by Coomassie gel staining (SDS-PAGE) and chemiluminescence (western blot against kappa casein and alpha-S1 casein, LSBio primary antibodies). Kappa casein expression in L. lactis was detected in the tested transformants by Coomassie stained protein gel and western blot.

### Example 9. Expression in L. lactis Via pH-Inducible System

Similar to the constructions above, casein protein constructions were created for alpha, beta, and kappa casein replacing the nisin promoter with the P170 promoter, a pH/lactate inducible promoter for L. lactis. Each of these constructs contained a secretion signal peptide.

Both alpha-S1 casein and kappa casein were detected in L. lactis upon secretion on western blot. Protein product accumulated intracellularly for alpha-S1 casein. Alpha-S1 casein secreted poorly, whereas kappa casein showed near-complete secretion of protein produced.

### Example 10. Expression in B. subtilis

Constructs Design, Cloning, and Transformation

Bovine alpha-S1 casein (variant C) protein coding sequence (without the native signal peptide) His-tagged C-terminally was codon-optimized for expression in Bacillus subtilis. Constructs were created with and without the codon-optimized signal peptide of amyQ, alpha-amylase Bacillus amyloliquefaciens which has been reported for the efficient secretion of recombinant proteins. Constructs were cloned through E. coli via Gibson cloning into transformation and expression IPTG-inducible vector pHT01 and confirmed via Sanger sequencing. pHT01 is an E. coli/B. subtilis shuttle vector that provides ampicillin resistance to E. coli and chloramphenicol resistance to B. subtilis. Positive clones were further transformed into chemically competent B. subtilis WB800N. Positive clones were confirmed via colony PCR and three positive clones were taken forward for the protein expression induction and analysis.

Protein Expression and Analysis

Individual colonies were grown at 37° C. in liquid culture and protein production was induced with IPTG for 1 hour, 2 hours, and 6 hours (control samples were left uninduced). Cells were then harvested by centrifugation, and TCA-precipitated supernatants and lysed cell pellets were analysed by Coomassie gel staining (SDS-PAGE) and chemiluminescence (Western Blot against His tag and alpha-S1 casein). Western blotting showed expression of the alpha-S1 casein in B. subtilis.

### Example 11. Expression in E. coli

Constructs Design, Cloning, and Transformation

Bovine alpha-S1 casein (variant C) protein coding sequence (without the native signal peptide) codon-optimized for Escherichia coli was cloned into IPTG-inducible commercially available pET vectors. Cloning was performed via Gibson reaction of DNA fragments and vector in such a way that only the protein coding sequence was left within the open reading frame. Gibson reactions were transformed into competent cells and confirmed by Sanger sequencing. Vectors were then transformed into chemically competent E. coli BL21(DE3) cells, or their derivatives (e.g. BL21-pLysS), and several single colonies were screened for expression.

Protein Expression, Analysis, and Purification

Individual colonies were grown at 37° C. in liquid culture, and protein production was induced with IPTG for 4 hours. Cells were then harvested by centrifugation, and lysed cell pellets were analysed by Coomassie gel staining (SDS-PAGE) and chemiluminescence (western blot against alpha-S1 casein). For protein purification, the insoluble fraction was removed by centrifugation and the soluble fraction was then precipitated with ammonium sulfate at room temperature and pelleted by centrifugation. The pellet was resuspended in urea, followed by dialysis against disodium phosphate. The insoluble proteins were removed by centrifugation, and the remaining contaminants were removed by precipitation with ethanol and ammonium acetate followed by centrifugation. The resulting alpha-S1 casein solution was concentrated using a centrifugal filtration unit and then dialyzed against disodium phosphate. Purified product was analysed on a Coomassie stained gel similarly to explained above. Alpha-S1 casein was expressed intracellularly in E. coli, successfully detected on Coomassie stained protein gel and purified.

### Example 12. Expression of Recombinant Alpha Casein and Kappa Casein

Constructs Design, Cloning, and Transformation

Alpha-S1 casein, kappa casein, and C-terminally truncated kappa casein protein coding sequence (without the native signal peptide, with or without an N-terminal His-tag or His-SUMO-tag) were each codon-optimized for Escherichia coli and were cloned individually into IPTG-inducible commercially available pET vectors. Cloning was performed via Gibson reaction of DNA fragments (IDT) and vector in such a way that only the protein coding sequence was left within the open reading frame. Gibson reactions were transformed into competent cells and confirmed by Sanger sequencing. Vectors were then transformed into chemically competent E. coli BL21(DE3) cells, or their derivatives (e.g. BL21(DE3) pLysS, Rosetta (DE3)), and several single colonies were screened for expression. The following expression vectors for producing alpha-S1 casein variants were created: pET-alpha-S1-casein(bovine), pET-6×His-alpha-S1-casein(bovine), pET-6×His-SUMO-alpha-S1-casein(bovine), pET-alpha-S1-casein(ovine), pET-6×His-alpha-S1-casein(ovine), pET-6×His-SUMO-alpha-S1-casein(ovine), pET-alpha-S1-casein(caprine), pET-6×His-alpha-S1-casein(caprine), pET-6×His-SUMO-alpha-S1-casein(caprine).

The following expression vectors for producing kappa casein variants were created: pET-kappa-casein(bovine), pET-6×His-kappa-casein(bovine), pET-6×His-SUMO-kappa-casein(bovine), pET-kappa-casein(ovine), pET-6×His-kappa-casein(ovine), pET-6×His-SUMO-kappa-casein(ovine), pET-kappa-casein(caprine), pET-6×His-kappa-casein(caprine), pET-6×His-SUMO-kappa-casein(caprine).

Protein Induction and Expression

An individual colony for each of the transformants was inoculated into TB medium containing 0.2% (v/v) glycerol and 100 μg/ml ampicillin or 50 μg/ml kanamycin. Cells were grown at 37° C. overnight in a shaking incubator. This overnight culture was used to inoculate 1 L of fresh TB medium containing 0.2% (v/v) glycerol and 100 μg/ml ampicillin or 50 μg/ml kanamycin, and cells were grown until OD600 reached ˜0.5-0.6, at which point isopropyl-thiogalactopyranoside (IPTG) was added to 0.5 mM. After four hours of incubation at 37° C., the cells were harvested by centrifugation and frozen at −80° C.

Protein Purification and Analysis

Frozen cell pellets were thawed on ice and resuspended in lysis buffer (40 mM Tris, pH 8, 0.3 M NaCl) supplemented with 2 mM Pefabloc, 0.1% (v/v) Triton X-100. The suspensions were lysed using a sonicator. The resulting total crude lysate was centrifuged at 10,000×g for 10 minutes at 4° C. to separate soluble and insoluble material. The soluble material was applied to equilibrated immobilized Ni-NTA Agarose resin, incubated for 1 hour on rotator at 4° C., and transferred to a gravity column to collect the beads. The resin beads were washed four times with 5-bed volume of wash buffer (40 mM Tris, pH 8, 0.3 M NaCl, 20 mM Imidazole) to remove non-specifically bound proteins. His-tagged proteins were eluted in 2-bed volume of elution buffer (40 mM Tris, pH 8, 0.3 M NaCl, 300 mM Imidazole). Following purification, protein samples were either dialyzed overnight in 10 mM K2HPO4 (if protein is not processed further) or in a buffer required for Ulp1 cleavage of SUMO tag. 6×His-SUMO-casein protein constructs were then used in proteolytic cleavage reaction with 6×His-Ulp1 at 4° C. overnight to generate untagged casein variants. Proteolyzed material was applied onto Ni-NTA Agarose resin in a “negative purification,” where the flow through and wash which contain the untagged casein variant were collected. Final untagged alpha-S1 casein variants and kappa casein variants were dialyzed overnight in 10 mM K2HPO4. Cell lysates as well as the purified products were analysed on a Coomassie stained SDS-PAGE.

What is claimed is:

1. A micelle composition comprising an alpha casein and a kappa casein, wherein at least one of the alpha casein and the kappa casein is a recombinant protein, wherein the alpha casein, the kappa casein, or both the alpha casein and the kappa casein comprise a non-native post-translational modification feature, and wherein the alpha casein and the kappa casein are associated in micelles.

2. The micelle composition of claim 1, wherein at least a portion of micelles of the micelle composition comprises cross-linked casein protein.

3. The micelle composition of claim 1 or claim 2, wherein the micelle composition comprises micelles having intra-micellar crosslinking.

4. The micelle composition of any one of claims 1-3, wherein at least a portion of the alpha casein and the kappa casein are in micellar form.

5. The micelle composition of any one of claims 1-4, wherein at least a portion of micelles of the micelle composition comprises intra-micellar crosslinking, and wherein a majority of the micelles are not comprised within an inter-micellar crosslinked structure.

6. The micelle composition of any one of claims 1-5, wherein the non-native post-translational modification feature comprises a reduction in phosphorylation, a lack of phosphorylation, or a modification of one or more sites of phosphorylation of the alpha casein.

7. The micelle composition of any one of claims 1-6, wherein the non-native post-translational modification feature comprises a reduction in glycosylation, a lack of glycosylation, or a modification of one or more sites of glycosylation of the kappa casein.

8. The micelle composition of any one of claims 1-7, wherein the alpha casein is a recombinant protein.

9. The micelle composition of any one of claims 1-7, wherein the kappa casein is a recombinant protein.

10. The micelle composition of any one of claims 1-7, wherein both the alpha casein and the kappa casein are recombinant proteins.

11. The micelle composition of any one of claims 1-10, wherein the alpha casein comprises a mixture of native alpha casein and one or more altered forms thereof.

12. The micelle composition of claim 11, wherein the one or more altered forms thereof is a truncated alpha casein (e.g., truncated relative to a native alpha casein).

13. The micelle composition of any one of claims 1-12, wherein the kappa casein comprises a mixture of a native kappa casein and one or more altered forms thereof.

14. The micelle composition of claim 13, wherein the one or more altered forms thereof is a truncated kappa casein (e.g., truncated relative to a native kappa casein).

15. The micelle composition of any one of claims 8-14, wherein the alpha casein, the kappa casein, or both the alpha casein and the kappa casein are produced in a recombinant host cell selected from the group consisting of: a microbial cell, a plant cell, and a mammalian cell; optionally, wherein the recombinant host cell is a microbial cell.

16. The micelle composition of claim 15, wherein the microbial cell is a bacterium.

17. The micelle composition of any one of claims 1-16, further comprising a beta casein or a derivative thereof.

18. The micelle composition of claim 17, further comprising a gamma casein.

19. The micelle composition of any one of claims 1-18, wherein the micelles do not comprise a beta casein or derivative thereof.

20. The micelle composition of any one of claims 1-19, wherein a ratio of the alpha casein to the kappa casein in the micelle composition is from about 1:1 to about 15:1.

21. The micelle composition of claim 20, wherein a ratio of the alpha casein to the kappa casein in the micelle composition is about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1.

22. The micelle composition of any one of claims 1-21, wherein the alpha casein comprises only alpha-S1 casein.

23. The micelle composition of any one of claims 1-21, wherein the alpha casein comprises only alpha-S2 casein.

24. The micelle composition of any one of claims 1-23, wherein the alpha casein comprises an amino acid sequence of cow, human, sheep, goat, buffalo, bison, horse, or camel alpha casein.

25. The micelle composition of any one of claims 1-24, wherein the alpha casein protein has an amino acid sequence comprising any one of SEQ ID NOs: 1-39 or 64-72 or a variant thereof having an amino acid sequence with at least 80% sequence identity to any one of SEQ ID NOs: 1-39 or 64-72.

26. The micelle composition of any one of claims 1-25, wherein the kappa casein comprises an amino acid sequence of cow, human, sheep, goat, buffalo, bison, horse, or camel kappa casein.

27. The micelle composition of any one of claims 1-26, wherein the kappa casein has an amino acid sequence comprising any one of SEQ ID NOs: 40-60, or a variant thereof having an amino acid sequence with at least 80% sequence identity to any one of SEQ ID NOs: 40-60.

28. The micelle composition of any one of claims 1-27, wherein the alpha casein and the kappa casein are from different mammalian species.

29. The micelle composition of claim 28, wherein the alpha casein comprises an amino acid sequence of bovine alpha casein, and the kappa casein comprises an amino acid sequence of sheep kappa casein.

30. The micelle composition of any one of claims 1-29, wherein the micelle composition comprises a population of micelles with an average or mean size from about 200 nm to about 400 nm.

31. The micelle composition of claim 30, wherein the micelle composition comprises a population of micelles with an average or mean size of about 300 nm.

32. The micelle composition of claim 30, wherein the micelle composition comprises a population of micelles with an average or mean size of about 200 nm.

33. The micelle composition of any one of claims 1-32, further comprising at least one salt selected from the group consisting of a calcium salt, a citrate salt, and a phosphate salt.

34. The micelle composition of any one of claims 1-33, wherein the micelle composition is susceptible to renneting.

35. The micelle composition of claim 34, wherein the micelle composition, after renneting, forms stable and strong curds (e.g., as measured by a tube inversion test).

36. A micelle-like composition comprising a kappa casein in the absence of alpha casein and beta casein, wherein the kappa casein forms a micelle-like structure.

37. The micelle-like composition of claim 36, wherein the kappa casein comprises intra-micellar crosslinking between kappa casein molecules.

38. The micelle-like composition of claim 36 or 37, wherein the kappa casein has an amino acid sequence comprising any one of SEQ ID NOs: 40-60, or a variant thereof comprising an amino acid sequence with at least 80% sequence identity to any one of SEQ ID NOs: 40-60.

39. The micelle-like composition of any one of claims 36-38, wherein the kappa casein comprises an amino acid sequence of cow, human, sheep, goat, buffalo, bison, horse, or camel kappa casein protein.

40. The micelle-like composition of claim 36 or 37, wherein the kappa casein has an amino acid sequence comprising any one of SEQ ID NOs: 43-45, or a variant thereof comprising an amino acid sequence with at least 80% sequence identity to any one of SEQ ID NOs: 43-45.

41. The micelle-like composition of claim 40, wherein the kappa casein comprises an amino acid sequence of sheep kappa casein protein.

42. The micelle-like composition of any one of claims 36-41, wherein the kappa casein comprises a mixture of a native kappa casein and one or more altered forms thereof.

43. The micelle-like composition of claim 42, wherein the one or more altered forms thereof is a truncated kappa casein (e.g., truncated relative to a native kappa casein).

44. The micelle-like composition of any one of claims 36-43, wherein the kappa casein comprises a first kappa casein protein and a second kappa casein protein.

45. The micelle-like composition of claim 44, wherein the first kappa casein protein and the second kappa casein protein are from different mammalian species.

46. The micelle-like composition of any one of claims 36-45, wherein the micelle-like composition comprises a population of micelle-like structures with an average or mean size from about 150 nm to about 700 nm.

47. The micelle-like composition of claim 46, wherein the micelle-like composition comprises a population of micelle-like structures with an average or mean size of about 400 nm.

48. The micelle-like composition of claim 46, wherein the micelle-like composition comprises a population of micelle-like structures with an average or mean size of about 100 nm to about 250 nm.

49. The micelle-like composition of claim 46, wherein the micelle-like composition comprises a population of micelle-like structures with an average or mean size of about 600 nm to about 700 nm.

50. The micelle-like composition of any one of claims 36-49, further comprising at least one salt selected from the group consisting of a calcium salt, a citrate salt, and a phosphate salt.

51. The micelle-like composition of any one of claims 36-50, wherein the micelle-like composition is susceptible to renneting.

52. The micelle-like composition of claim 51, wherein the micelle-like composition, after renneting, forms stable and strong curds (e.g., as measured by a tube inversion test).

53. A dairy-like product comprising the micelle composition according to any one of claims 1-35 or the micelle-like composition of any one of claims 36-53.

54. The dairy-like product of claim 53, wherein the micelle composition or micelle-like composition does not include any additional dairy protein.

55. The dairy-like product of claim 53 or 54, incorporated into an edible composition, wherein the edible composition does not include any animal-obtained dairy protein.

56. The dairy-like product of any one of claims 53-55, wherein the dairy-like product is selected from the group consisting of milk, yogurt, curd, cheese, cream, and ice cream.

57. The dairy-like product of any one of claims 53-56, wherein the dairy-like product is curd.

58. The dairy-like product of any one of claims 53-55, wherein the dairy-like product comprises a cheese selected from the group consisting of a soft cheese, a hard cheese, a pasta filata cheese, and an aged cheese.

59. The dairy-like product of claim 58, wherein the cheese has a fat content from about 0% to about 50% and the fat is not an animal-obtained fat.

60. The dairy-like product of claim 58 or claim 59, wherein the cheese has a sugar content from about 0% to about 10% and the sugar is derived from a plant-based source.

61. The dairy-like product of any one of claims 58-60, wherein the cheese is selected from the group consisting of pasta filata-like cheese, paneer, cream cheese, and cottage cheese.

62. The dairy-like product of any one of claims 58-60, wherein the cheese is an aged or matured cheese selected from the group consisting of cheddar, Swiss, gouda, brie, camembert, feta, halloumi, edam, manchego, colby, muenster, blue cheese, or parmesan.

63. The dairy-like product of any one of claims 58-60, wherein the cheese is mozzarella.

64. The dairy-like product of any one of claims 58-63, wherein the moisture retention of the cheese is from about 30% to about 80%.

65. The dairy-like product of any one of claims 58-64, wherein the cheese is capable of one or more of stretching when heated, melting when heated, or browning when heated.

66. The dairy-like product of any one of claims 58-65, wherein the texture of the cheese is comparable to an animal-obtained dairy cheese.

67. The dairy-like product of any one of claims 58-66, wherein the hardness of the cheese is comparable to an animal-obtained dairy cheese.

68. The dairy-like product of any one of claims 58-66, wherein the hardness of the cheese is reduced as compared to an animal-obtained dairy cheese.

69. The dairy-like product of any one of claims 58-68, wherein the melt of the cheese is comparable to an animal-obtained dairy cheese.

70. The dairy-like product of any one of claims 58-68, wherein the melt of the cheese is improved compared to an animal-obtained dairy cheese.

71. The dairy-like product of any one of claims 58-70, wherein the stretch of the cheese is comparable to an animal-obtained dairy cheese.

72. The dairy-like product of any one of claims 58-70, wherein the stretch of the cheese is improved compared to an animal-obtained dairy cheese.

73. The dairy-like product of any one of claims 58-72, wherein the dairy-like product comprises the micelle-like composition of claim 37, and the yield of the cheese is improved as compared to a comparable dairy-like product without the intra-micellar crosslinking between kappa casein molecules.

74. The dairy-like product of any one of claims 58-73, wherein the dairy-like product comprises the micelle-like composition of claim 37, and the melt of the cheese is improved as compared to a comparable dairy-like product without the intra-micellar crosslinking between kappa casein molecules.

75. The dairy-like product of any one of claims 58-74, wherein the dairy-like product comprises the micelle-like composition of claim 37, and the stretch of the cheese is improved as compared to a comparable dairy-like product without the intra-micellar crosslinking between kappa casein molecules.

76. A powder comprising the micelle composition of any one of claims 1-35 or the micelle-like composition of any one of claims 36-52.

77. The powder of claim 76, wherein the casein content of the powder is from about 50% to about 90%.

78. A method of making a dairy-like ingredient comprising:
(a) providing an alpha casein and a kappa casein, wherein the alpha casein, the kappa casein, or both the alpha casein and the kappa casein comprise a non-native post-translational modification feature;
(b) inducing micelle formation; and
(c) providing a crosslinking agent under conditions for inducing intra-micellar crosslinking,
wherein the method produces micelles comprising the alpha casein and the kappa casein in a form suitable for a dairy-like ingredient.

79. A method of making a dairy-like ingredient comprising:
(a) providing an alpha casein, wherein the alpha casein comprises a non-native posttranslational modification feature;
(b) providing a crosslinking agent under conditions for crosslinking the alpha casein; and
(c) mixing kappa casein with the crosslinked alpha casein under conditions to induce micelle formation,
wherein the method produces micelles comprising the alpha casein and the kappa casein in a form suitable for a dairy-like ingredient.

80. The method of claim 78, wherein the alpha casein and kappa casein are incubated together prior to adding the crosslinking agent.

81. The method of claim 80, wherein the crosslinking agent is added from about 30 minutes to about 24 hours after the alpha casein and kappa casein are incubated together.

82. The method of claim 80, wherein the crosslinking agent is added from about 1 hour to about 12 hours after the alpha casein and kappa casein are incubated together.

83. The method of claim 78 or 79, wherein the crosslinking agent is added prior to the step of inducing micelle formation.

84. The method of claim 78, wherein the crosslinking agent is added subsequent to the step of inducing micelle formation.

85. The method of any one of claims 78-84, wherein the crosslinking agent is a transglutaminase.

86. The method of any one of claims 78-85, wherein the non-native posttranslational modification feature comprises a reduction in phosphorylation, a lack of phosphorylation, or a modification of one or more sites of phosphorylation on the alpha casein.

87. The method of any one of claims 78-86, wherein the non-native post-translational modification feature comprises a reduction in glycosylation, a lack of glycosylation, or a modification of one or more sites of glycosylation on the kappa casein.

88. The method of any one of claims 78-87, wherein the method further comprises producing the alpha casein, the kappa casein, or both in a recombinant host cell selected from the group consisting of a microbial cell, a plant cell, and a mammalian cell; optionally, wherein the recombinant host cell is a microbial cell.

89. The method of claim 88, wherein the recombinant host cell is a microbial cell.

90. The method of claim 89, wherein the microbial cell is selected from the group consisting of Lactococci sp., Lactococcus lactis, Bacillus subtilis, Bacillus amyloliquefaciens, Bacillus licheniformis, Bacillus megaterium, Mycobacterium smegmatis, Rhodococcus erythropolis, Corynebacterium glutamicum, Lactobacilli sp., Lactobacillus fermentum, Lactobacillus casei, Lactobacillus acidophilus, Lactobacillus plantarum, Synechocystis sp. 6803, and Escherichia coli.

91. The method of any one of claims 78-90, wherein the dairy-like ingredient is susceptible to renneting.

92. The method of any one of claims 78-91, wherein the conditions to induce micelle formation include the addition of a salt.

93. The method of any one of claims 78-92, wherein the micelles are comprised in a liquid colloid.

94. The method of claim 93, further comprising the step of forming a dairy-like product from the liquid colloid.

95. The method of claim 94, wherein the dairy-like product comprises milk, cream, curd, cheese, yogurt, or ice cream.

96. The method of claim 94, further comprising subjecting the liquid colloid to a first condition to form coagulates.

97. The method of claim 96, wherein the first condition is the addition of acid or acidification of the liquid colloid with a microorganism.

98. The method of claim 96 or 97, wherein the method further comprises subjecting the coagulates to a hot water treatment and optionally stretching to form a filata type cheese.

99. The method of claim 96 or 97, wherein the method further comprises subjecting the coagulates to a renneting agent to form a rennetted curd.

100. The method of claim 99, wherein the renneting agent is a microbially-derived chymosin enzyme.

101. The method of claim 99 or 100, wherein the method further comprises cooking, aging, and maturing the rennetted curd to form an aged or matured cheese-like composition.

102. The method of claim 99 or 100, wherein the method further comprises subjecting the rennetted curd to a hot water treatment and optionally stretching to form a filata-type cheese.

103. The method of claim 93, further comprising forming a yogurt from the liquid colloid.

104. The method of claim 103, wherein forming the yogurt comprises optionally heating and then cooling the liquid colloid, and acidifying the liquid colloid with a microorganism.

105. The method of claim 104, wherein the microorganism comprises one or more of Lactobacillus delbrueckii subsp. bulgaricus, Streptococcus thermophilus, a Lactobacilli, or a Bifzdobacteria species.

106. The method of any one of claims 78-105, wherein the micelles do not include beta casein protein.

107. The method of any one of claims 78-106, wherein the dairy-like ingredient does not include any additional dairy protein.

108. The method of any one of claims 78-106, wherein the dairy-like ingredient does not include any animal-obtained dairy protein.

109. The method of claim 94, wherein the dairy-like product comprises a fat, a sugar, a flavoring, or a colorant.

110. The method of any one of claims 78-109, wherein the dairy-like ingredient is in a powder form.

111. The method of claim 110, wherein the method further comprises drying, lyophilizing, drum drying, or spray-drying to produce the powder form.

112. The method of any one of claims 78-111, wherein the ratio of alpha casein to kappa casein is from about 1:1 to about 15:1.

113. The method of claim 112, wherein the ratio of alpha casein to kappa protein is about 1:1, about 2:1, about 3:1, about 4:1, about 5:1, about 6:1, about 7:1, about 8:1, about 9:1, about 10:1, about 11:1, about 12:1, about 13:1, about 14:1, or about 15:1.

114. The method of any one of claims 78-113, wherein the alpha casein comprises only alpha-S1 casein.

115. The method of any one of claims 78-113, wherein the alpha casein comprises only alpha-S2 casein.

116. The method of any one of claims 78-115, wherein the alpha casein has an amino acid sequence comprising any one of SEQ ID NOs: 1-39 or 64-72, or a variant thereof comprising an amino acid sequence with at least 80% sequence identity to any one of SEQ ID NOs: 1-39 or 64-72.

117. The method of any one of claims 78-116, wherein the alpha casein comprises an amino acid sequence of cow, human, sheep, goat, buffalo, bison, horse, or camel alpha casein.

118. The method of any one of claims 78-117, wherein the kappa casein has an amino acid sequence comprising any one of SEQ ID NOs: 40-60, or a variant thereof comprising an amino acid sequence with at least 80% sequence identity to any one of SEQ ID NOs: 40-60.

119. The method of any one of claims 78-118, wherein the kappa casein comprises an amino acid sequence of cow, human, sheep, goat, buffalo, bison, horse, or camel kappa casein protein.

120. The method of any one of claims 78-117, wherein the kappa casein has an amino acid sequence comprising any one of SEQ ID NOs: 43-45, or a variant thereof comprising an amino acid sequence with at least 80% sequence identity to any one of SEQ ID NOs: 43-45.

121. The method of claim 120, wherein the kappa casein comprises an amino acid sequence of sheep kappa casein protein.

122. The method of any one of claims 78-121, wherein the alpha casein comprises a mixture of native alpha casein and one or more altered forms thereof.

123. The method of claim 122, wherein the one or more altered forms thereof is a truncated alpha casein (e.g., truncated relative to a native alpha casein).

124. The method of any one of claims 78-123, wherein the kappa casein comprises a mixture of a native kappa casein and one or more altered forms thereof.

125. The method of claim 124, wherein the one or more altered forms thereof is a truncated kappa casein (e.g., truncated relative to a native kappa casein).

126. The method of any one of claims 78-125, wherein the alpha casein and the kappa casein are from different mammalian species.

127. The method of claim 126, wherein the alpha casein comprises an amino acid sequence of bovine alpha casein, and the kappa casein comprises an amino acid sequence of sheep kappa casein.

128. A coagulated composition formed by the method of any one of claims 78-127.

129. A renneted curd composition formed by the method of any one of claims 78-127.

130. A dairy-like composition formed by the method of any one of claims 78-127.

131. The dairy-like composition of claim 130, wherein the dairy-like composition is selected from the group consisting of milk, cream, curd, cheese, yogurt, and ice cream.

132. The dairy-like composition of claim 130, wherein the dairy-like composition is selected from the group consisting of pasta filata-like cheese, paneer, cream cheese, cottage cheese, cheddar, Swiss, gouda, and mozzarella.

133. A method of making a dairy-like ingredient comprising providing a kappa casein in the absence of any alpha casein or beta casein under conditions such that the kappa casein forms a micelle-like structure in a form suitable for a dairy-like ingredient.

134. The method of claim 133, further comprising, providing a crosslinking agent under conditions for crosslinking the kappa casein.

135. The method of claim 133 or 134, wherein the micelle-like structure comprises intra-micellar crosslinking between kappa casein molecules.

136. The method of any one of claims 133-135, wherein the kappa casein has an amino acid sequence comprising any one of SEQ ID NOs: 40-60, or a variant thereof comprising an amino acid sequence with at least 80% sequence identity to any one of SEQ ID NOs: 40-60.

137. The method of any one of claims 133-136, wherein the kappa casein comprises an amino acid sequence of cow, human, sheep, goat, buffalo, bison, horse, or camel kappa casein.

138. The method of any one of claims 133-137, wherein the micelle-like composition comprises a population of micelle-like structures with an average or mean size from about 300 nm to about 500 nm.

139. The method of claim 138, wherein the micelle-like composition comprises a population of micelle-like structures with an average or mean size of about 100 nm to about 250 nm.

140. The method of claim 138, wherein the micelle-like composition comprises a population of micelle-like structures with an average or mean size of about 600 nm to about 700 nm.

141. The method of claim 138, wherein the micelle-like composition comprises a population of micelle-like structures with an average or mean size of about 400 nm.

142. The method of any one of claims 112-117, wherein the cross-linking agent is inactivated after formation of the micelle-like structures.

143. The method of any one of claims 134-142, wherein the cross-linking agent comprises transglutaminase.

144. The method of any one of claims 134-143, wherein the kappa casein has an amino acid sequence comprising any one of SEQ ID NOs: 40-60, or a variant thereof comprising an amino acid sequence with at least 80% sequence identity to any one of SEQ ID NOs: 40-60.

145. The method of any one of claims 134-144, wherein the kappa casein comprises an amino acid sequence of cow, human, sheep, goat, buffalo, bison, horse, or camel kappa casein protein.

146. The method of any one of claims 134-143, wherein the kappa casein has an amino acid sequence comprising any one of SEQ ID NOs: 43-45, or a variant thereof comprising an amino acid sequence with at least 80% sequence identity to any one of SEQ ID NOs: 43-45.

147. The method of claim 146, wherein the kappa casein comprises an amino acid sequence of sheep kappa casein protein.

148. The method of any one of claims 134-147, wherein the kappa casein comprises a mixture of a native kappa casein and one or more altered forms thereof.

149. The method of claim 148, wherein the one or more altered forms thereof is a truncated kappa casein (e.g., truncated relative to a native kappa casein).

150. The method of any one of claims 134-149, wherein the kappa casein comprises a first kappa casein protein and a second kappa casein protein.

151. The method of claim 150, wherein the first kappa casein protein and the second kappa casein protein are from different mammalian species.

152. A dairy-like composition formed by the method of any one of claims 133-151.

153. The dairy-like composition of claim 152, wherein the dairy-like composition comprises a pasta filata cheese.


SEQUENCE LISTING (72 sequences)

SEQ ID NO: 1 - protein, 214 aa, Unknown
  Bos sp.
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 2 - protein, 199 aa, Unknown
  Bos sp.
RPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 3 - protein, 200 aa, Unknown
  Bos sp.
MRPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 4 - protein, 214 aa, Unknown
  Ovis sp.
MKLLILTCLVAVALARPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 5 - protein, 199 aa, Unknown
  Ovis sp.
RPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 6 - protein, 200 aa, Unknown
  Ovis sp.
MRPKHPIKHQGLSSEVLNENLLRFVVAPFPEVFRKENINELSKDIGSESIEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKITMPLW

SEQ ID NO: 7 - protein, 214 aa, Unknown
  Capra sp.
MKLLILTCLVAVALARPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 8 - protein, 199 aa, Unknown
  Capra sp.
RPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 9 - protein, 200 aa, Unknown
  Capra sp.
MRPKHPINHRGLSPEVPNENLLRFVVAPFPEVFRKENINELSKDIGSESTEDQAMEDAKQMKAGSSSSSEEIVPNSAEQKYIQKEDVPSERYLGYLEQLLRLKKYNVPQLEIVPKSAEEQLHSMKEGNPAHQKQPMIAVNQELAYFYPQLFRQFYQLDAYPSGAWYYLPLGTQYTDAPSFSDIPNPIGSENSGKTTMPLW

SEQ ID NO: 10 - protein, 214 aa, Unknown
  Bubalus sp.
MKLLILTCLVAVALARPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 11 - protein, 199 aa, Unknown
  Bubalus sp.
RPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 12 - protein, 200 aa, Unknown
  Bubalus sp.
MRPKQPIKHQGLPQGVLNENLLRFFVAPFPEVFGKEKVNELSTDIGSESTEDQAMEDIKQMEAESISSSEEIVPISVEQKHIQKEDVPSERYLGYLEQLLRLKKYNVPQLEIVPNLAEEQLHSMKEGIHAQQKEPMIGVNQELAYFYPQLFRQFYQLDAYPSGAWYYVPLGTQYPDAPSFSDIPNPIGSENSEKTTMPLW

SEQ ID NO: 13 - protein, 212 aa, Unknown
  Equus sp.
MKLLILTCLVAVALARPKLPHRQPEIIQNEQDSREKVLKERKFPSFALEYINELNRQRELLKEKQKDEHKEYLIEDPEQQESSSTSSSEEVVPINTEQKRIPREDMLYQHTLEQLRRLSKYNQLQLQAIHAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 14 - protein, 197 aa, Unknown
  Equus sp.
RPKLPHRQPEIIQNEQDSREKVLKERKFPSFALEYINELNRQRELLKEKQKDEHKEYLIEDPEQQESSSTSSSEEVVPINTEQKRIPREDMLYQHTLEQLRRLSKYNQLQLQAIHAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 15 - protein, 198 aa, Unknown
  Equus sp.
MRPKLPHRQPEIIQNEQDSREKVLKERKFPSFALEYINELNRQRELLKEKQKDEHKEYLIEDPEQQESSSTSSSEEVVPINTEQKRIPREDMLYQHTLEQLRRLSKYNQLQLQAIHAQEQLIRMKENSQRKPMRVVNQEQAYFYLEPFQPSYQLDVYPYAAWFHPAQIMQHVAYSPFHDTAKLIASENSEKTDIIPEW

SEQ ID NO: 16 - protein, 230 aa, Unknown
  Camelus sp.
MKLLILTCLVAVALARPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 17 - protein, 215 aa, Unknown
  Camelus sp.
RPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 18 - protein, 216 aa, Unknown
  Camelus sp.
MRPKYPLRYPEVFQNEPDSIEEVLNKRKILELAVVSPIQFRQENIDELKDTRNEPTEDHIMEDTERKESGSSSSEEVVSSTTEQKDILKEDMPSQRYLEELHRLNKYKLLQLEAIRDQKLIPRVKLSSHPYLEQLYRINEDNHPQLGEPVKVVTQEQAYFHLEPFPQFFQLGASPYVAWYYPPQVMQYIAHPSSYDTPEGIASEDGGKTDVMPQWW

SEQ ID NO: 19 - protein, 185 aa, Homo sapiens
MRLLILTCLVAVALARPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 20 - protein, 170 aa, Homo sapiens
RPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 21 - protein, 171 aa, Homo sapiens
MRPKLPLRYPERLQNPSESSEPIPLESREEYMNGMNRQRNILREKQTDEIKDTRNESTQNCVVAEPEKMESSISSSSEEMSLSKCAEQFCRLNEYNQLQLQAAHAQEQIRRMNENSHVQVPFQQLNQLAAYPYAVWYYPQIMQYVPFPPFSDISNPTAHENYEKNNVMLQW

SEQ ID NO: 22 - protein, 222 aa, Unknown
  Bos sp.
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 23 - protein, 207 aa, Unknown
  Bos sp.
KNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 24 - protein, 208 aa, Unknown
  Bos sp.
MKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 25 - protein, 223 aa, Unknown
  Ovis sp.
MKFFIFTCLLAVALAKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 26 - protein, 208 aa, Unknown
  Ovis sp.
KHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 27 - protein, 209 aa, Unknown
  Ovis sp.
MKHKMEHVSSSEEPINISQEIYKQEKNMAIHPRKEKLCTTSCEEVVRNADEEEYSIRSSSEESAEVAPEEVKITVDDKHYQKALNEINQFYQKFPQYLQYLYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 28 - protein, 223 aa, Unknown
  Capra sp.
MKFFIFTCLLAVALAKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 29 - protein, 208 aa, Unknown
  Capra sp.
KHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 30 - protein, 209 aa, Unknown
  Capra sp.
MKHKMEHVSSSEEPINIFQEIYKQEKNMAIHPRKEKLCTTSCEEVVRNANEEEYSIRSSSEESAEVAPEEIKITVDDKHYQKALNEINQFYQKFPQYLQYPYQGPIVLNPWDQVKRNAGPFTPTVNREQLSTSEENSKKTIDMESTEVFTKKTKLTEEEKNRLNFLKKISQYYQKFAWPQYLKTVDQHQKAMKPWTQPKTNAIPYVRYL

SEQ ID NO: 31 - protein, 222 aa, Unknown
  Bubalus sp.
MKFFIFTCLLAVALAKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 32 - protein, 207 aa, Unknown
  Bubalus sp.
KHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 33 - protein, 208 aa, Unknown
  Bubalus sp.
MKHTMEHVSSSEESIISQETYKQEKNMAIHPSKENLCSTFCKEVIRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEDKNRLNFLKKISQHYQKFAWPQYLKTVYQYQKAMKPWTQPKTNVIPYVRYL

SEQ ID NO: 34 - protein, 214 aa, Unknown
  Equus sp.
MKFFIFTCLLAVALAKHNMEHRSSSEDSVNISQEKFKQEKYVVIPTSKESICSTSCEEATRNINEMESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 35 - protein, 199 aa, Unknown
  Equus sp.
KHNMEHRSSSEDSVNISQEKFKQEKYVVIPTSKESICSTSCEEATRNINEMESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 36 - protein, 200 aa, Unknown
  Equus sp.
MKHNMEHRSSSEDSVNISQEKFKQEKYVVIPTSKESICSTSCEEATRNINEMESAKFPTEREEKEVEEKHHLKQLNKINQFYEKLNFLQYLQALRQPRIVLTPWDQTKTGDSPFIPIVNTEQLFTSEEIPKKTVDMESTEVVTEKTELTEEEKNYLKLLYYEKFTLPQYFKIVRQHQTTMDPRSHRKTNSYQIIPVLRYF

SEQ ID NO: 37 - protein, 193 aa, Unknown
  Camelus sp.
MKFFIFTCLLAVVLAKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 38 - protein, 178 aa, Unknown
  Camelus sp.
KHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 39 - protein, 179 aa, Unknown
  Camelus sp.
MKHEMDQGSSSEESINVSQQKFKQVKKVAIHPSKEDICSTFCEEAVRNIKEVESAEVPTENKISQFYQKWKFLQYLQALHQGQIVMNPWDQGKTRAYPFIPTVNTEQLSISEESTEVPTEESTEVFTKKTELTEEEKDHQKFLNKIYQYYQTFLWPEYLKTVYQYQKTMTPWNHIKRYF

SEQ ID NO: 40 - protein, 190 aa, Unknown
  Bos sp.
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 41 - protein, 169 aa, Unknown
  Bos sp.
QEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 42 - protein, 170 aa, Unknown
  Bos sp.
MQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 43 - protein, 192 aa, Unknown
  Ovis sp.
MMKSFFLVVTILALTLPFLGAQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 44 - protein, 171 aa, Unknown
  Ovis sp.
QEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 45 - protein, 172 aa, Unknown
  Ovis sp.
MQEQNQEQRICCEKDERFFDDKIAKYIPIQYVLSRYPSYGLNYYQQRPVALINNQFLPYPYYAKPVAVRSPAQTLQWQVLPNAVPAKSCQDQPTAMARHPHPHLSFMAIPPKKDQDKTEIPAINTIASAEPTVHSTPTTEAVVNAVDNPEASSESIASAPETNTAQVTSTEV

SEQ ID NO: 46 - protein, 192 aa, Unknown
  Capra sp.
MMKSFFLVVTILALTLPFLGAQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 47 - protein, 171 aa, Unknown
  Capra sp.
QEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 48 - protein, 172 aa, Unknown
  Capra sp.
MQEQNQEQPICCEKDERFFDDKIAKYIPIQYVLSRYPSYGLNYYQQRPVALINNQFLPYPYYAKPVAVRSPAQTLQWQVLPNTVPAKSCQDQPTTLARHPHPHLSFMAIPPKKDQDKTEVPAINTIASAEPTVHSTPTTEAIVNTVDNPEASSESIASASETNTAQVTSTEV

SEQ ID NO: 49 - protein, 190 aa, Unknown
  Bubalus sp.
MMKSFFLVVTILALTLPFLGAQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 50 - protein, 169 aa, Unknown
  Bubalus sp.
QEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 51 - protein, 170 aa, Unknown
  Bubalus sp.
MQEQNQEQPIRCEKEERFFNDKIAKYIPIQYVLSRYPSYGLNYYQQKPVALINNQFLPYPYYAKPAAVRSPAQILQWQVLPNTVPAKSCQAQPTTMTRHPHPHLSFMAIPPKKNQDKTEIPTINTIVSVEPTSTPTTEAIENTVATLEASSEVIESVPETNTAQVTSTVV

SEQ ID NO: 52 - protein, 185 aa, Unknown
  Equus sp.
MKSFFLVVNILALTLPFLGAEVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 53 - protein, 165 aa, Unknown
  Equus sp.
EVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 54 - protein, 166 aa, Unknown
  Equus sp.
MEVQNQEQPTCHKNDERFFDLKTVKYIPIYYVLNSSPRYEPIYYQHRLALLINNQHMPYQYYARPAAVRPHVQIPQWQVLPNIYPSTVVRHPCPHPSFIAIPPKKLQEITVIPKINTIATVEPTPIPTPEPTVNNAVIPDASSEFIIASTPETTTVPVTSPVVQKL

SEQ ID NO: 55 - protein, 182 aa, Unknown
  Camelus sp.
MKSFFLVVTILALTLPFLGAEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 56 - protein, 162 aa, Unknown
  Camelus sp.
EVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 57 - protein, 163 aa, Unknown
  Camelus sp.
MEVQNQEQPTCFEKVERLLNEKTVKYFPIQFVQSRYPSYGINYYQHRLAVPINNQFIPYPNYAKPVAIRLHAQIPQCQALPNIDPPTVERRPRPRPSFIAIPPKKTQDKTVNPAINTVATVEPPVIPTAEPAVNTVVIAEASSEFITTSTPETTTVQITSTEI

SEQ ID NO: 58 - protein, 182 aa, Homo sapiens
MKSFLLVVNALALTLPFLAVEVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 59 - protein, 162 aa, Homo sapiens
EVQNQKQPACHENDERPFYQKTAPYVPMYYVPNSYPYYGTNLYQRRPAIAINNPYVPRTYYANPAVVRPHAQIPQRQYLPNSHPPTVVRRPNLHPSFIAIPPKKIQDKIIIPTINTIATVEPTPAPATEPTVDSVVTPEAFSESIITSTPETTTVAVTPPTA

SEQ ID NO: 60 - protein, 162 aa, Homo sapiens; identical to SEQ ID NO: 59

SEQ ID NO: 61 - protein, 224 aa, Unknown
  Bos sp.
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 62 - protein, 209 aa, Unknown
  Bos sp.
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 63 - protein, 210 aa, Unknown
  Bos sp.
MRELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 64 - protein, 177 aa, Bos taurus
FFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 65 - protein, 178 aa, Bos taurus
MFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 66 - protein, 176 aa, Bos taurus
FVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 67 - protein, 177 aa, Bos taurus
MFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 68 - protein, 175 aa, Bos taurus
VAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 69 - protein, 176 aa, Bos taurus
MVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 70 - protein, 174 aa, Bos taurus
APFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 71 - protein, 175 aa, Bos taurus
MAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSGKTTMPLW

SEQ ID NO: 72 - protein, 140 aa, Bos taurus
MEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW